(19) United States
(12) Patent Application Publication
(10) Pub. No.: US 2005/0196829 A1
(43) Pub. Date: Sep. 8, 2005

(54) Haplotypes of the FCER1A gene

(76) Inventors: Anne Chew, Brookline, MA (US); R. Rex Denton, Madison, CT (US); Amy Duda, Milano (IT); Stefanie E. Kliem, Oberursel (DE); Elizabeth M. Brown, Creedmor, NC (US); Krishnan Nandabalan, Guilford, CT (US); J. Claiborne Stephens, Guilford, CT (US)

Correspondence Address: GENAISSANCE PHARMACEUTICALS, 5 SCIENCE PARK, NEW HAVEN, CT, 06511, US

(21) Appl. No.: 10/073,735

(22) Filed: Feb. 11, 2002

Related U.S. Application Data
(63) Continuation-in-part of application No. PCT/US00/21097, filed on Aug. 2, 2000.
(60) Provisional application No. 60/147,860, filed on Aug. 9, 1999.

(51) Int. Cl. C12Q 1/68; C07H 21/04; C12P 21/02; C12N 5/06

(52) U.S. Cl. 435/69.1; 435/6; 435/325; 435/320.1; 530/350; 536/23.2

(57) ABSTRACT

Novel genetic variants of the Fc Fragment Of Ige, High Affinity I, Receptor For; Alpha Polypeptide (FCER1A) gene are described. Various genotypes, haplotypes, and haplotype pairs that exist in the general United States population are disclosed for the FCER1A gene. Compositions and methods for haplotyping and/or genotyping the FCER1A gene in an individual are also disclosed. Polynucleotides defined by the haplotypes disclosed herein are also described.

### RELATED APPLICATIONS

This application is a continuation-in-part of International Application PCT/US00/21097 filed Aug. 2, 2000, which claims the benefit of U.S. Provisional Application Ser. No. 60/147,860 filed Aug. 9, 1999.

### FIELD OF THE INVENTION

This invention relates to variation in genes that encode pharmaceutically-important proteins. In particular, this invention provides genetic variants of the human Fc fragment of IgE, high affinity I, receptor for; alpha polypeptide (FCER1A) gene and methods for identifying which variant(s) of this gene is/are possessed by an individual.

### BACKGROUND OF THE INVENTION

Current methods for identifying pharmaceuticals to treat disease often start by identifying, cloning, and expressing an important target protein related to the disease. A determination of whether an agonist or antagonist is needed to produce an effect that may benefit a patient with the disease is then made. Then, vast numbers of compounds are screened against the target protein to find new potential drugs. The desired outcome of this process is a lead compound that is specific for the target, thereby reducing the incidence of the undesired side effects usually caused by activity at non-intended targets. The lead compound identified in this screening process then undergoes further in vitro and in vivo testing to determine its absorption, disposition, metabolism and toxicological profiles. Typically, this testing involves use of cell lines and animal models with limited, if any, genetic diversity.

What this approach fails to consider, however, is that natural genetic variability exists between individuals in any and every population with respect to pharmaceutically-important proteins, including the protein targets of candidate drugs, the enzymes that metabolize these drugs and the proteins whose activity is modulated by such drug targets. Subtle alteration(s) in the primary nucleotide sequence of a gene encoding a pharmaceutically-important protein may be manifested as significant variation in expression, structure and/or function of the protein. Such alterations may explain the relatively high degree of uncertainty inherent in the treatment of individuals with a drug whose design is based upon a single representative example of the target or enzyme(s) involved in metabolizing the drug. For example, it is well-established that some drugs frequently have lower efficacy in some individuals than others, which means such individuals and their physicians must weigh the possible benefit of a larger dosage against a greater risk of side effects. Also, there is significant variation in how well people metabolize drugs and other exogenous chemicals, resulting in substantial interindividual variation in the toxicity and/or efficacy of such exogenous substances (Evans et al., 1999, Science 286:487-491). This variability in efficacy or toxicity of a drug in genetically-diverse patients makes many drugs ineffective or even dangerous in certain groups of the population, leading to the failure of such drugs in clinical trials or their early withdrawal from the market even though they could be highly beneficial for other groups in the population. This problem significantly increases the time and cost of drug discovery and development, which is a matter of great public concern.

It is well-recognized by pharmaceutical scientists that considering the impact of the genetic variability of pharmaceutically-important proteins in the early phases of drug discovery and development is likely to reduce the failure rate of candidate and approved drugs (Marshall A 1997 Nature Biotech 15:1249-52; Kleyn P W et al. 1998 Science 281: 1820-21; Kola I 1999 Curr Opin Biotech 10:589-92; Hill AVS et al. 1999 in Evolution in Health and Disease Stearns S S (Ed.) Oxford University Press, New York, pp 62-76; Meyer U. A. 1999 in Evolution in Health and Disease Stearns S S (Ed.) Oxford University Press, New York, pp 41-49; Kalow W et al. 1999 Clin. Pharm. Therap. 66:445-7; Marshall, E 1999 Science 284:406-7; Judson R et al. 2000 Pharmacogenomics 1:1-12; Roses A D 2000 Nature 405:857-65). However, in practice this has been difficult to do, in large part because of the time and cost required for discovering the amount of genetic variation that exists in the population (Chakravarti A 1998 Nature Genet 19:216-7; Wang D G et al 1998 Science 280:1077-82; Chakravarti A 1999 Nat Genet 21:56-60 (suppl); Stephens J C 1999 Mol. Diagnosis 4:309-317; Kwok P Y and Gu S 1999 Mol. Med. Today 5:538-43; Davidson S 2000 Nature Biotech 18:1134-5).

The standard for measuring genetic variation among individuals is the haplotype, which is the ordered combination of polymorphisms in the sequence of each form of a gene that exists in the population. Because haplotypes represent the variation across each form of a gene, they provide a more accurate and reliable measurement of genetic variation than individual polymorphisms. For example, while specific variations in gene sequences have been associated with a particular phenotype such as disease susceptibility (Roses A D supra; Ulbrecht M et al. 2000 Am J Respir Crit Care Med 161: 469-74) and drug response (Wolfe C R et al. 2000 BMJ 320:987-90; Dahl B S 1997 Acta Psychiatr Scand 96 (Suppl 391): 14-21), in many other cases an individual polymorphism may be found in a variety of genomic backgrounds, i.e., different haplotypes, and therefore shows no definitive coupling between the polymorphism and the causative site for the phenotype (Clark A G et al. 1998 Am J Hum Genet 63:595-612; Ulbrecht M et al. 2000 supra; Drysdale et al. 2000 PNAS 97:10483-10488). Thus, there is an unmet need in the pharmaceutical industry for information on what haplotypes exist in the population for pharmaceutically-important genes. Such haplotype information would be useful in improving the efficiency and output of several steps in the drug discovery and development process, including target validation, identifying lead compounds, and early phase clinical trials (Marshall et al., supra).

One pharmaceutically-important gene for the treatment of inflammatory disorders, such as allergies, asthma, and autoimmune diseases is the Fc fragment of IgE, high affinity I, receptor for; alpha polypeptide (FCER1A) gene or its encoded product. FCER1A, also known as immunoglobulin E Receptor 1 alpha subunit gene (IgERA), belongs to the family of antibody Fc receptors that play an important role in the immune response by coupling the specificity of secreted antibodies to a variety of cell types of the immune system. Fc receptors initiate immune system respones during normal immunity, allergies, antibody-mediated tumor recognition, and autoimmune diseases such as arthritis. FCER1A mediates IgE-dependent peripheral and systemic anaphylaxis, regulates IgE metabolism, and plays a role in the growth and differentiation of various cell typess of the immune system.

FCER1A initiates the immediate hypersensitivity response from mast cells and basophils. Evidence indicates this receptor is involved in antiparasitic reactions from platelets and eosinophils, and in antigen delivery to dendritic cells for major histocompatibility complex class II presentation pathways activating T cells. Moreover, FCER1A exerts a regulatory effect on IgE production, as well as differentiation and growth of mast cells and B-lymphocytes.

Stimulation of FCER1A initiates a cascade of events resulting in a number of cellular events, one of which is the release of inflammatory mediators, such as histamine, from mast cells. In addition, cytokines are released, particularly interleukin 4 (IL-4), which is critical in B-cell switching and IgE synthesis pathways, as well as in the control of FCER1A synthesis. Induction of expression of other mast cell surface receptors, such as CD40, involved in immune cell growth and differentiation as well as IgE metabolism, also transpires. Other factors whose expression and/or secretion are regulated by FCER1A include interleukin 6 (IL-6), tissue necrosis factor alpha (TNFα), RANTES, and serotonin, among others.

FCER1A is a tetrameric transmembrane protein consisting of an alpha, beta, and two disulfide-bonded gamma polypeptides. The alpha subunit, IGERA, binds IgE with high affinity (K_{d} ˜10.9-10.10M) and can be secreted as a soluble IgE-binding fragment. The gamma subunit, FCER1G, mediates receptor assembly and signal transduction, and is a common component of other Fc receptors, including the high-affinity and low-affinity IgG receptors, and the TCR/CD3 Tcell receptor complex. The role of the beta subunit, FCER1B, is more enigmatic, although it is also involved in signal transduction and receptor autophosphorylation. FCER1B is essential for full activation of mast cells for the allergic response and is an amplifier of signaling from the gamma subunit.

The Fc fragment of IgE, high affinity I, receptor for; alpha polypeptide gene is located on chromosome 1q21-q23 and contains 5 exons that encode a 257 amino acid protein. A reference sequence for the FCER1A gene is shown in the contiguous lines of FIG. 1 (Genaissance Reference No. 3179200; SEQ ID NO: 1). Reference sequences for the coding sequence (GenBank Accession No. NM_{—}002001.1) and protein are shown in FIGS. 2 (SEQ ID NO: 2) and 3 (SEQ ID NO: 3), respectively.

Interest in discovering polymorphisms in genes encoding subunits of FCER1A arises from the role played by IgE in atopy. Atopy is a common familial disorder caused by genetic and environmental factors. It is characterized by exaggerated T helper cell type II lymphocyte responses to common allergens, such as pollens and dust mites, and includes sustained, enhanced production of IgE. Allergy, asthma, rhinitis, and eczema are atopic hypersensitivity diseases. IgE binds to the high affinity IgE receptor presented on mucosal mast cells and basophils. IgE binding of allergens activates the receptor and initiates a cascade, leading to cellular release of inflammatory mediators. Dysregulation of the normal immediate hypersensitivity response results in abnormally high and sustained IgE serum levels, which leads to mucosal inflammation. Atopy is detected by elevated total serum IgE levels, positive skin prick tests to common allergens, and specific serum IgE against these allergens. All three have been strongly correlated with each other and the presence of the symptoms of allergic reaction such as wheezing, coughing, sneezing, and nasal blockage.

Approximately 20% of the world population is affected by allergies, with over 50% of western populations testing positive to skin prick tests of one or more common allergens. Up to 10% of children suffer from atopic asthma, accounting for approximately one-third of pediatric emergency room visits in the United States. While a single genetic determinant is unlikely to be the causative factor in asthma, allergy, or other atopic diseases, therapeutics aimed at the obligatory binding of IgE to FCER1A for initiation of the allergic response could provide a single treatment for the various manifestations of atopic hypersensitivity.

Few published studies have been performed to identify polymorphisms at the FCER1A locus. One known polymorphism at the FCER1A locus consists of an RsaI restriction fragment length polymorphism (RFLP) detected in genomic DNA using a cDNA probe (Tepler et al., supra).

Because of the potential for variation in the FCER1A gene to affect the expression and function of the encoded protein, it would be useful to know whether polymorphisms exist in the FCER1A gene, as well as how such polymorphisms are combined in different copies of the gene. Such information could be applied for studying the biological function of FCER1A as well as in identifying drugs targeting this protein for the treatment of disorders related to its abnormal expression or function.

### SUMMARY OF THE INVENTION

Accordingly, the inventors herein have discovered 22 novel polymorphic sites in the FCER1A gene. These polymorphic sites (PS) correspond to the following nucleotide positions in FIG. 1: 586 (PS1), 657 (PS2), 906 (PS3), 913 (PS4), 1077 (PS5), 1468 (PS6), 1474 (PS7), 1610 (PS8), 2422 (PS9), 2738 (PS10), 2789 (PS11), 2934 (PS12), 3000 (PS13), 3044 (PS14), 4552 (PS15), 4822 (PS16), 4999 (PS17), 5077 (PS18), 6535 (PS19), 6625 (PS20), 6650 (PS21) and 6714 (PS22). The polymorphisms at these sites are thymine or guanine at PS 1, thymine or cytosine at PS2, thymine or cytosine at PS3, adenine or thymine at PS4, cytosine or adenine at PS5, thymine or cytosine at PS6, cytosine or adenine at PS7, cytosine or thymine at PS8, adenine or guanine at PS9, adenine or guanine at PS 10, guanine or adenine at PS11, thymine or cytosine at PS 12, guanine or adenine at PS 13, guanine or adenine at PS 14, guanine or adenine at PS 15, cytosine or thymine at PS16, cytosine or thymine at PS17, thymine or cytosine at PS18, cytosine or adenine at PS19, thymine or cytosine at PS20, adenine or guanine at PS21 and guanine or adenine at PS22. In addition, the inventors have determined the identity of the alleles at these sites in a human reference population of 79 unrelated individuals self-identified as belonging to one of four major population groups: African descent, Asian, Caucasian and Hispanic/Latino. From this information, the inventors deduced a set of haplotypes and haplotype pairs for PS 1-PS22 in the FCER1A gene, which are shown below in Tables 5 and 4, respectively. Each of these FCER1A haplotypes constitutes a code that defines the variant nucleotides that exist in the human population at this set of polymorphic sites in the FCER1A gene. Thus each FCER1A haplotype also represents a naturally-occurring isoform (also referred to herein as an “isogene”) of the FCER1A gene. The frequency of each haplotype and haplotype pair within the total reference population and within each of the four major population groups included in the reference population was also determined.

Thus, in one embodiment, the invention provides a method, composition and kit for genotyping the FCER1A gene in an individual. The genotyping method comprises identifying the nucleotide pair that is present at one or more polymorphic sites selected from the group consisting of PS1, PS2, PS3, PS4, PS5, PS6, PS7, PS8, PS9, PS 10, PS11, PS12, PS13, PS14, PS15, PS16, PS17, PS18, PS 19, PS20, PS21 and PS22 in both copies of the FCER1A gene from the individual. A genotyping composition of the invention comprises an oligonucleotide probe or primer which is designed to specifically hybridize to a target region containing, or adjacent to, one of these novel FCER1A polymorphic sites. A genotyping kit of the invention comprises a set of oligonucleotides designed to genotype each of these novel FCER1A polymorphic sites. The genotyping method, composition, and kit are useful in determining whether an individual has one of the haplotypes in Table 5 below or has one of the haplotype pairs in Table 4 below.

The invention also provides a method for haplotyping the FCER1A gene in an individual. In one embodiment, the haplotyping method comprises determining, for one copy of the FCER1A gene, the identity of the nucleotide at one or more polymorphic sites selected from the group consisting of PS1, PS2, PS3, PS4, PS5, PS6, PS7, PS8, PS9, PS10, PS11, PS12, PS13, PS14, PS15, PS16, PS17, PS18, PS19, PS20, PS21 and PS22. In another embodiment, the haplotyping method comprises determining whether one copy of the individual's FCER1A gene is defined by one of the FCER1A haplotypes shown in Table 5, below, or a sub-haplotype thereof. In a preferred embodiment, the haplotyping method comprises determining whether both copies of the individual's FCER1A gene are defined by one of the FCER1A haplotype pairs shown in Table 4 below, or a sub-haplotype pair thereof. Establishing the FCER1A haplotype or haplotype pair of an individual is useful for improving the efficiency and reliability of several steps in the discovery and development of drugs for treating diseases associated with FCER1A activity, e.g., inflammatory disorders, such as allergies, asthma, and autoimmune diseases.

For example, the haplotyping method can be used by the pharmaceutical research scientist to validate FCER1A as a candidate target for treating a specific condition or disease predicted to be associated with FCER1A activity. Determining for a particular population the frequency of one or more of the individual FCER1A haplotypes or haplotype pairs described herein will facilitate a decision on whether to pursue FCER1A as a target for treating the specific disease of interest. In particular, if variable FCER1A activity is associated with the disease, then one or more FCER1A haplotypes or haplotype pairs will be found at a higher frequency in disease cohorts than in appropriately genetically matched controls. Conversely, if each of the observed FCER1A haplotypes are of similar frequencies in the disease and control groups, then it may be inferred that variable FCER1A activity has little, if any, involvement with that disease. In either case, the pharmaceutical research scientist can, without a priori knowledge as to the phenotypic effect of any FCER1A haplotype or haplotype pair, apply the information derived from detecting FCER1A haplotypes in an individual to decide whether modulating FCER1A activity would be useful in treating the disease.

The claimed invention is also useful in screening for compounds targeting FCER1A to treat a specific condition or disease predicted to be associated with FCER1A activity. For example, detecting which of the FCER1A haplotypes or haplotype pairs disclosed herein are present in individual members of a population with the specific disease of interest enables the pharmaceutical scientist to screen for a compound(s) that displays the highest desired agonist or antagonist activity for each of the FCER1A isoforms present in the disease population, or for only the most frequent FCER1A isoforms present in the disease population. Thus, without requiring any a priori knowledge of the phenotypic effect of any particular FCER1A haplotype or haplotype pair, the claimed haplotyping method provides the scientist with a tool to identify lead compounds that are more likely to show efficacy in clinical trials.

Haplotyping the FCER1A gene in an individual is also useful in the design of clinical trials of candidate drugs for treating a specific condition or disease predicted to be associated with FCER1A activity. For example, instead of randomly assigning patients with the disease of interest to the treatment or control group as is typically done now, determining which of the FCER1A haplotype(s) disclosed herein are present in individual patients enables the pharmaceutical scientist to distribute FCER1A haplotypes and/or haplotype pairs evenly to treatment and control groups, thereby reducing the potential for bias in the results that could be introduced by a larger frequency of a FCER1A haplotype or haplotype pair that is associated with response to the drug being studied in the trial, even if this association was previously unknown. Thus, by practicing the claimed invention, the scientist can more confidently rely on the information learned from the trial, without first determining the phenotypic effect of any FCER1A haplotype or haplotype pair.

In another embodiment, the invention provides a method for identifying an association between a trait and a FCER1A genotype, haplotype, or haplotype pair for one or more of the novel polymorphic sites described herein. The method comprises comparing the frequency of the FCER1A genotype, haplotype, or haplotype pair in a population exhibiting the trait with the frequency of the FCER1A genotype or haplotype in a reference population. A higher frequency of the FCER1A genotype, haplotype, or haplotype pair in the trait population than in the reference population indicates the trait is associated with the FCER1A genotype, haplotype, or haplotype pair. In preferred embodiments, the trait is susceptibility to a disease, severity of a disease, the staging of a disease or response to a drug. In a particularly preferred embodiment, the FCER1A haplotype is selected from the haplotypes shown in Table 5, or a sub-haplotype thereof. Such methods have applicability in developing diagnostic tests and therapeutic treatments for inflammatory disorders, such as allergies, asthma, and autoimmune diseases.

In yet another embodiment, the invention provides an isolated polynucleotide comprising a nucleotide sequence which is a polymorphic variant of a reference sequence for the FCER1A gene or a fragment thereof. The reference sequence comprises the contiguous sequences shown in FIG. 1 and the polymorphic variant comprises at least one polymorphism selected from the group consisting of guanine at PS1, cytosine at PS2, cytosine at PS3, thymine at PS4, adenine at PS5, cytosine at PS6, adenine at PS7, thymine at PS8, guanine at PS9, guanine at PS10, adenine at PS11, cytosine at PS12, adenine at PS13, adenine at PS14, adenine at PS15, thymine at PS16, thymine at PS17, cytosine at PS18, adenine at PS19, cytosine at PS20, guanine at PS21 and adenine at PS22.

A particularly preferred polymorphic variant is an isogene of the FCER1A gene. A FCER1A isogene of the invention comprises thymine or guanine at PS1, thymine or cytosine at PS2, thymine or cytosine at PS3, adenine or thymine at PS4, cytosine or adenine at PS5, thymine or cytosine at PS6, cytosine or adenine at PS7, cytosine or thymine at PS8, adenine or guanine at PS9, adenine or guanine at PS10, guanine or adenine at PS11, thymine or cytosine at PS12, guanine or adenine at PS13, guanine or adenine at PS14, guanine or adenine at PS15, cytosine or thymine at PS16, cytosine or thymine at PS17, thymine or cytosine at PS18, cytosine or adenine at PS19, thymine or cytosine at PS20, adenine or guanine at PS21 and guanine or adenine at PS22. The invention also provides a collection of FCER1A isogenes, referred to herein as a FCER1A genome anthology.

In another embodiment, the invention provides a polynucleotide comprising a polymorphic variant of a reference sequence for a FCER1A cDNA or a fragment thereof. The reference sequence comprises SEQ ID NO:2 (FIG. 2) and the polymorphic cDNA comprises at least one polymorphism selected from the group consisting of guanine at a position corresponding to nucleotide 251, adenine at a position corresponding to nucleotide 302, thymine at a position corresponding to nucleotide 530 and adenine at a position corresponding to nucleotide 741. A particularly preferred polymorphic cDNA variant comprises the coding sequence of a FCER1A isogene defined by haplotypes 7, 10, 12, 16, 17, and 19.

Polynucleotides complementary to these FCER1A genomic and cDNA variants are also provided by the invention. It is believed that polymorphic variants of the FCER1A gene will be useful in studying the expression and function of FCER1A, and in expressing FCER1A protein for use in screening for candidate drugs to treat diseases related to FCER1A activity.

In other embodiments, the invention provides a recombinant expression vector comprising one of the polymorphic genomic and cDNA variants operably linked to expression regulatory elements as well as a recombinant host cell transformed or transfected with the expression vector. The recombinant vector and host cell may be used to express FCER1A for protein structure analysis and drug binding studies.

In yet another embodiment, the invention provides a polypeptide comprising a polymorphic variant of a reference amino acid sequence for the FCER1A protein. The reference amino acid sequence comprises SEQ ID NO:3 (FIG. 3) and the polymorphic variant comprises at least one variant amino acid selected from the group consisting of arginine at a position corresponding to amino acid position 84, asparagine at a position corresponding to amino acid position 101, methionine at a position corresponding to amino acid position 177 and lysine at a position corresponding to amino acid position 247. A polymorphic variant of FCER1A is useful in studying the effect of the variation on the biological activity of FCER1A as well as on the binding affinity of candidate drugs targeting FCER1A for the treatment of inflammatory disorders, such as allergies, asthma, and autoimmune diseases.

The present invention also provides antibodies that recognize and bind to the above polymorphic FCER1A protein variant. Such antibodies can be utilized in a variety of diagnostic and prognostic formats and therapeutic methods.

The present invention also provides nonhuman transgenic animals comprising one or more of the FCER1A polymorphic genomic variants described herein and methods for producing such animals. The transgenic animals are useful for studying expression of the FCER1A isogenes in vivo, for in vivo screening and testing of drugs targeted against FCER1A protein, and for testing the efficacy of therapeutic agents and compounds for inflammatory disorders, such as allergies, asthma, and autoimmune diseases in a biological system.

The present invention also provides a computer system for storing and displaying polymorphism data determined for the FCER1A gene. The computer system comprises a computer processing unit; a display; and a database containing the polymorphism data. The polymorphism data includes one or more of the following: the polymorphisms, the genotypes, the haplotypes, and the haplotype pairs identified for the FCER1A gene in a reference population. In a preferred embodiment, the computer system is capable of producing a display showing FCER1A haplotypes organized according to their evolutionary relationships.

### BRIEF DESCRIPTION OF THE DRAWINGS

FIG. 1 illustrates a reference sequence for the FCER1A gene (Genaissance Reference No. 3179200; contiguous lines), with the start and stop positions of each region of coding sequence indicated with a bracket ([or]) and the numerical position below the sequence and the polymorphic site(s) and polymorphism(s) identified by Applicants in a reference population indicated by the variant nucleotide positioned below the polymorphic site in the sequence. SEQ ID NO:1 is equivalent to FIG. 1, with the two alternative allelic variants of each polymorphic site indicated by the appropriate nucleotide symbol (R=G or A, Y=T or C, M=A or C, K=G or T, S=G or C, and W=A or T; WIPO standard ST.25). SEQ ID NO:114 is a modified version of SEQ ID NO:1 that shows the context sequence of each polymorphic site, PS1-PS22, in a uniform format to facilitate electronic searching. For each polymorphic site, SEQ ID NO:114 contains a block of 60 bases of the nucleotide sequence encompassing the centrally-located polymorphic site at the 30^{th} position, followed by 60 bases of unspecified sequence to represent that each PS is separated by genomic sequence whose composition is defined elsewhere herein.

FIG. 2 illustrates a reference sequence for the FCER1A coding sequence (contiguous lines; SEQ ID NO:2), with the polymorphic site(s) and polymorphism(s) identified by Applicants in a reference population indicated by the variant nucleotide positioned below the polymorphic site in the sequence.

FIG. 3 illustrates a reference sequence for the FCER1A protein (contiguous lines; SEQ ID NO:3), with the variant amino acid(s) caused by the polymorphism(s) of FIG. 2 positioned below the polymorphic site in the sequence.

### DESCRIPTION OF THE PREFERRED EMBODIMENTS

The present invention is based on the discovery of novel variants of the FCER1A gene. As described in more detail below, the inventors herein discovered 22 isogenes of the FCER1A gene by characterizing the FCER1A gene found in genomic DNAs isolated from an Index Repository that contains immortalized cell lines from one chimpanzee and 93 human individuals. The human individuals included a reference population of 79 unrelated individuals self-identified as belonging to one of four major population groups: Caucasian (21 individuals), African descent (20 individuals), Asian (20 individuals), or Hispanic/Latino (18 individuals). To the extent possible, the members of this reference population were organized into population subgroups by their self-identified ethnogeographic origin as shown in Table 1 below. In addition, the Index Repository contains three unrelated indigenous American Indians (one from each of North, Central and South America), one three-generation Caucasian family (from the CEPH Utah cohort) and one two-generation African-American family.

TABLE 1
Population Groups in the Index Repository

| | | No. of |
|---|---|---|
| Population Group | Population Subgroup | Individuals |
| African descent | | 20 |
| | Sierra Leone | 1 |
| Asian | | 20 |
| | Burma | 1 |
| | China | 3 |
| | Japan | 6 |
| | Korea | 1 |
| | Philippines | 5 |
| | Vietnam | 4 |
| Caucasian | | 21 |
| | British Isles | 3 |
| | British Isles/Central | 4 |
| | British Isles/Eastern | 1 |
| | Central/Eastern | 1 |
| | Eastern | 3 |
| | Central/Mediterranean | 1 |
| | Mediterranean | 2 |
| | Scandinavian | 2 |
| Hispanic/Latino | | 18 |
| | Caribbean | 8 |
| | Caribbean (Spanish Descent) | 2 |
| | Central American (Spanish Descent) | 1 |
| | Mexican American | 4 |
| | South American (Spanish Descent) | 3 |

The FCER1A isogenes present in the human reference population are defined by haplotypes for 22 polymorphic sites in the FCER1A gene, all of which are believed to be novel. The novel FCER1A polymorphic sites identified by the inventors are referred to as PS1-PS22 to designate the order in which they are located in the gene (see Table 3 below). Using the genotypes identified in the Index Repository for PS1-PS22 and the methodology described in the Examples below, the inventors herein also determined the pair of haplotypes for the FCER1A gene present in individual human members of this repository. The human genotypes and haplotypes found in the repository for the FCER1A gene include those shown in Tables 4 and 5, respectively. The polymorphism and haplotype data disclosed herein are useful for validating whether FCER1A is a suitable target for drugs to treat inflammatory disorders, such as allergies, asthma, and autoimmune diseases, screening for such drugs and reducing bias in clinical trials of such drugs.

In the context of this disclosure, the following terms shall be defined as follows unless otherwise indicated:

Allele—A particular form of a genetic locus, distinguished from other forms by its particular nucleotide sequence.

Candidate Gene—A gene which is hypothesized to be responsible for a disease, condition, or the response to a treatment, or to be correlated with one of these.

Gene—A segment of DNA that contains the coding sequence for a protein, wherein the segment may include promoters, exons, introns, and other untranslated regions that control expression.

Genotype—An unphased 5′ to 3′ sequence of nucleotide pair(s) found at one or more polymorphic sites in a locus on a pair of homologous chromosomes in an individual. As used herein, genotype includes a full-genotype and/or a sub-genotype as described below.

Full-genotype—The unphased 5′ to 3′ sequence of nucleotide pairs found at all polymorphic sites examined herein in a locus on a pair of homologous chromosomes in a single individual.

Sub-genotype—The unphased 5′ to 3′ sequence of nucleotides seen at a subset of the polymorphic sites examined herein in a locus on a pair of homologous chromosomes in a single individual.

Genotyping—A process for determining a genotype of an individual.

Haplotype—A 5′ to 3′ sequence of nucleotides found at one or more polymorphic sites in a locus on a single chromosome from a single individual. As used herein, haplotype includes a full-haplotype and/or a sub-haplotype as described below.

Full-haplotype—The 5′ to 3′ sequence of nucleotides found at all polymorphic sites examined herein in a locus on a single chromosome from a single individual.

Sub-haplotype—The 5′ to 3′ sequence of nucleotides seen at a subset of the polymorphic sites examined herein in a locus on a single chromosome from a single individual.

Haplotype pair—The two haplotypes found for a locus in a single individual.

Haplotyping—A process for determining one or more haplotypes in an individual and includes use of family pedigrees, molecular techniques and/or statistical inference.

Haplotype data—Information concerning one or more of the following for a specific gene: a listing of the haplotype pairs in each individual in a population; a listing of the different haplotypes in a population; frequency of each haplotype in that or other populations, and any known associations between one or more haplotypes and a trait.

Isoform—A particular form of a gene, mRNA, cDNA, coding sequence or the protein encoded thereby, distinguished from other forms by its particular sequence and/or structure.

Isogene—One of the isoforms (e.g., alleles) of a gene found in a population. An isogene (or allele) contains all of the polymorphisms present in the particular isoform of the gene.

Isolated—As applied to a biological molecule such as RNA, DNA, oligonucleotide, or protein, isolated means the molecule is substantially free of other biological molecules such as nucleic acids, proteins, lipids, carbohydrates, or other material such as cellular debris and growth media. Generally, the term “isolated” is not intended to refer to a complete absence of such material or to absence of water, buffers, or salts, unless they are present in amounts that substantially interfere with the methods of the present invention.

Locus—A location on a chromosome or DNA molecule corresponding to a gene or a physical or phenotypic feature, where physical features include polymorphic sites.

Naturally-occurring—A term used to designate that the object it is applied to, e.g., naturally-occurring polynucleotide or polypeptide, can be isolated from a source in nature and which has not been intentionally modified by man.

Nucleotide pair—The nucleotides found at a polymorphic site on the two copies of a chromosome from an individual.

Phased—As applied to a sequence of nucleotide pairs for two or more polymorphic sites in a locus, phased means the combination of nucleotides present at those polymorphic sites on a single copy of the locus is known.

Polymorphic site (PS)—A position on a chromosome or DNA molecule at which at least two alternative sequences are found in a population.

Polymorphic variant (variant)—A gene, mRNA, cDNA, polypeptide, protein or peptide whose nucleotide or amino acid sequence varies from a reference sequence due to the presence of a polymorphism in the gene.

Polymorphism—The sequence variation observed in an individual at a polymorphic site. Polymorphisms include nucleotide substitutions, insertions, deletions and microsatellites and may, but need not, result in detectable differences in gene expression or protein function.

Polymorphism data—Information concerning one or more of the following for a specific gene: location of polymorphic sites; sequence variation at those sites; frequency of polymorphisms in one or more populations; the different genotypes and/or haplotypes determined for the gene; frequency of one or more of these genotypes and/or haplotypes in one or more populations; any known association(s) between a trait and a genotype or a haplotype for the gene.

Polymorphism Database—A collection of polymorphism data arranged in a systematic or methodical way and capable of being individually accessed by electronic or other means.

Polynucleotide—A nucleic acid molecule comprised of single-stranded RNA or DNA or comprised of complementary, double-stranded DNA.

Population Group—A group of individuals sharing a common ethnogeographic origin.

Reference Population—A group of subjects or individuals who are predicted to be representative of the genetic variation found in the general population. Typically, the reference population represents the genetic variation in the population at a certainty level of at least 85%, preferably at least 90%, more preferably at least 95% and even more preferably at least 99%.

Single Nucleotide Polymorphism (SNP)—Typically, the specific pair of nucleotides observed at a single polymorphic site. In rare cases, three or four nucleotides may be found.

Subject—A human individual whose genotypes or haplotypes or response to treatment or disease state are to be determined.

Treatment—A stimulus administered internally or externally to a subject.

Unphased—As applied to a sequence of nucleotide pairs for two or more polymorphic sites in a locus, unphased means the combination of nucleotides present at those polymorphic sites on a single copy of the locus is not known.

As discussed above, information on the identity of genotypes and haplotypes for the FCER1A gene of any particular individual as well as the frequency of such genotypes and haplotypes in any particular population of individuals is useful for a variety of drug discovery and development applications. Thus, the invention also provides compositions and methods for detecting the novel FCER1A polymorphisms, haplotypes and haplotype pairs identified herein.

The compositions comprise at least one oligonucleotide for detecting the variant nucleotide or nucleotide pair located at a novel FCER1A polymorphic site in one copy or two copies of the FCER1A gene. Such oligonucleotides are referred to herein as FCER1A haplotyping oligonucleotides or genotyping oligonucleotides, respectively, and collectively as FCER1A oligonucleotides. In one embodiment, a FCER1A haplotyping or genotyping oligonucleotide is a probe or primer capable of hybridizing to a target region that contains, or that is located close to, one of the novel polymorphic sites described herein.

As used herein, the term “oligonucleotide” refers to a polynucleotide molecule having less than about 100 nucleotides. A preferred oligonucleotide of the invention is 10 to 35 nucleotides long. More preferably, the oligonucleotide is between 15 and 30, and most preferably, between 20 and 25 nucleotides in length. The exact length of the oligonucleotide will depend on many factors that are routinely considered and practiced by the skilled artisan. The oligonucleotide may be comprised of any phosphorylation state of ribonucleotides, deoxyribonucleotides, and acyclic nucleotide derivatives, and other functionally equivalent derivatives. Alternatively, oligonucleotides may have a phosphate-free backbone, which may be comprised of linkages such as carboxymethyl, acetamidate, carbamate, polyamide (peptide nucleic acid (PNA)) and the like (Varma, R. in Molecular Biology and Biotechnology, A Comprehensive Desk Reference, Ed. R. Meyers, VCH Publishers, Inc. (1995), pages 617-620). Oligonucleotides of the invention may be prepared by chemical synthesis using any suitable methodology known in the art, or may be derived from a biological sample, for example, by restriction digestion. The oligonucleotides may be labeled, according to any technique known in the art, including use of radiolabels, fluorescent labels, enzymatic labels, proteins, haptens, antibodies, sequence tags and the like.

Haplotyping or genotyping oligonucleotides of the invention must be capable of specifically hybridizing to a target region of a FCER1A polynucleotide. Preferably, the target region is located in a FCER1A isogene. As used herein, specific hybridization means the oligonucleotide forms an anti-parallel double-stranded structure with the target region under certain hybridizing conditions, while failing to form such a structure when incubated with another region in the FCER1A polynucleotide or with a non-FCER1A polynucleotide under the same hybridizing conditions. Preferably, the oligonucleotide specifically hybridizes to the target region under conventional high stringency conditions. The skilled artisan can readily design and test oligonucleotide probes and primers suitable for detecting polymorphisms in the FCER1A gene using the polymorphism information provided herein in conjunction with the known sequence information for the FCER1A gene and routine techniques.

A nucleic acid molecule such as an oligonucleotide or polynucleotide is said to be a “perfect” or “complete” complement of another nucleic acid molecule if every nucleotide of one of the molecules is complementary to the nucleotide at the corresponding position of the other molecule. A nucleic acid molecule is “substantially complementary” to another molecule if it hybridizes to that molecule with sufficient stability to remain in a duplex form under conventional low-stringency conditions. Conventional hybridization conditions are described, for example, by Sambrook J. et al., in Molecular Cloning, A Laboratory Manual, 2^{nd} Edition, Cold Spring Harbor Press, Cold Spring Harbor, N.Y. (1989) and by Haymes, B. D. et al. in Nucleic Acid Hybridization, A Practical Approach, IRL Press, Washington, D.C. (1985). While perfectly complementary oligonucleotides are preferred for detecting polymorphisms, departures from complete complementarity are contemplated where such departures do not prevent the molecule from specifically hybridizing to the target region. For example, an oligonucleotide primer may have a non-complementary fragment at its 5′ end, with the remainder of the primer being complementary to the target region. Alternatively, non-complementary nucleotides may be interspersed into the probe or primer as long as the resulting probe or primer is still capable of specifically hybridizing to the target region.

Preferred haplotyping or genotyping oligonucleotides of the invention are allele-specific oligonucleotides. As used herein, the term allele-specific oligonucleotide (ASO) means an oligonucleotide that is able, under sufficiently stringent conditions, to hybridize specifically to one allele of a gene, or other locus, at a target region containing a polymorphic site while not hybridizing to the corresponding region in another allele(s). As understood by the skilled artisan, allele-specificity will depend upon a variety of readily optimized stringency conditions, including salt and formamide concentrations, as well as temperatures for both the hybridization and washing steps. Examples of hybridization and washing conditions typically used for ASO probes are found in Kogan et al., “Genetic Prediction of Hemophilia A” in PCR Protocols, A Guide to Methods and Applications, Academic Press, 1990 and Ruaño et al., 87 Proc. Natl. Acad. Sci. USA 6296-6300, 1990. Typically, an ASO will be perfectly complementary to one allele while containing a single mismatch for another allele.

Allele-specific oligonucleotides of the invention include ASO probes and ASO primers. ASO probes which usually provide good discrimination between different alleles are those in which a central position of the oligonucleotide probe aligns with the polymorphic site in the target region (e.g., approximately the 7^{th} or 8^{th} position in a 15mer, the 8^{th} or 9^{th} position in a 16mer, and the 10^{th} or 11^{th} position in a 20mer). An ASO primer of the invention has a 3′ terminal nucleotide, or preferably a 3′ penultimate nucleotide, that is complementary to only one nucleotide of a particular SNP, thereby acting as a primer for polymerase-mediated extension only if the allele containing that nucleotide is present. ASO probes and primers hybridizing to either the coding or noncoding strand are contemplated by the invention. ASO probes and primers listed below use the appropriate nucleotide symbol (R=G or A, Y=T or C, M=A or C, K=G or T, S=G or C, and W=A or T; WIPO standard ST.25) at the position of the polymorphic site to represent that the ASO contains either of the two alternative allelic variants observed at that polymorphic site.

A preferred ASO probe for detecting FCER1A gene polymorphisms comprises a nucleotide sequence, listed 5′ to 3′, selected from the group consisting of:

| | | |
|---|---|---|
| TGAAATAKCAGATTT | (SEQ ID NO:4) | and its complement, |
| ATTCTGCYCTCCCTT | (SEQ ID NO:5) | and its complement, |
| GATATGAYACAGAAA | (SEQ ID NO:6) | and its complement, |
| TACAGAAWACATTTC | (SEQ ID NO:7) | and its complement, |
| AATTACCMCTCCCAG | (SEQ ID NO:8) | and its complement, |
| ACTAATGYATCCTCT | (SEQ ID NO:9) | and its complement, |
| GTATCCTMTCTGGAC | (SEQ ID NO:10) | and its complement, |
| TAATGAGYATGAATC | (SEQ ID NO:11) | and its complement, |
| AATCAAARCAGGGTC | (SEQ ID NO:12) | and its complement, |
| AATGCCARATTTGAA | (SEQ ID NO:13) | and its complement, |
| AATGAGARTGAACCT | (SEQ ID NO:14) | and its complement, |
| AGGCCTCYCATTTTT | (SEQ ID NO:15) | and its complement, |
| TTTGGGARGCTGAGG | (SEQ ID NO:16) | and its complement, |
| ACCATCCRGCTAACA | (SEQ ID NO:17) | and its complement, |
| ATGCGTGRCTCTCTT | (SEQ ID NO:18) | and its complement, |
| TACTGTAYGGGCAAA | (SEQ ID NO:19) | and its complement, |
| AGCCTACYAGACTTG | (SEQ ID NO:20) | and its complement, |
| ATGGTGAYAGTAATA | (SEQ ID NO:21) | and its complement, |
| TTCTGAAMCCACATC | (SEQ ID NO:22) | and its complement, |
| CAATTGCYACTCAAT | (SEQ ID NO:23) | and its complement, |
| AGCTTGCRATATACA | (SEQ ID NO:24) | and its complement, |
| and | | |
| TGAAACTRGTTAAGT | (SEQ ID NO:25) | and its complement. |

A preferred ASO primer for detecting FCER1A gene polymorphisms comprises a nucleotide sequence, listed 5′ to 3′, selected from the group consisting of:

| | |
|---|---|
| AATAAATGAAATAKC; | (SEQ ID NO:26) |
| CTAAATAAATCTGMT; | (SEQ ID NO:27) |
| TGTTTTATTCTGCYC; | (SEQ ID NO:28) |
| GGATGCAAGGGAGRG; | (SEQ ID NO:29) |
| TAACCAGATATGAYA; | (SEQ ID NO:30) |
| AAATGTTTTCTGTRT; | (SEQ ID NO:31) |
| ATATGATACAGAAWA; | (SEQ ID NO:32) |
| CAGAAGGAAATGTWT; | (SEQ ID NO:33) |
| AGATTCAATTACCMC; | (SEQ ID NO:34) |
| GCCTCCCTGGGAGKG; | (SEQ ID NO:35) |
| CTGGACACTAATGYA; | (SEQ ID NO:36) |
| GTCCAGAGAGGATRC; | (SEQ ID NO:37) |
| ACTAATGTATCCTMT; | (SEQ ID NO:38) |
| GCAAAAGTCCAGAKA; | (SEQ ID NO:39) |
| GCTTTCTAATGAGYA; | (SEQ ID NO:40) |
| GGAACAGATTCATRC; | (SEQ ID NO:41) |
| CCTAGAAATCAAARC; | (SEQ ID NO:42) |
| TGATAAGACCCTGYT; | (SEQ ID NO:43) |
| ATTGTGAATGCCARA; | (SEQ ID NO:44) |
| ACTGTCTTCAAATYT; | (SEQ ID NO:45) |
| CAAGTTAATGAGART; | (SEQ ID NO:46) |
| GTACACAGGTTCAYT; | (SEQ ID NO:47) |
| GATTCAAGGCCTCYC; | (SEQ ID NO:48) |
| GGTCTTAAAAATGRG; | (SEQ ID NO:49) |
| CAGCACTTTGGGARG; | (SEQ ID NO:50) |
| CACCTGCCTCAGCYT; | (SEQ ID NO:51) |
| ATCGAGACCATCCRG; | (SEQ ID NO:52) |
| TCACCATGTTAGCYG; | (SEQ ID NO:53) |
| TGCTCTATGCGTGRC; | (SEQ ID NO:54) |
| AGAGAAAAGAGAGYC; | (SEQ ID NO:55) |
| ACCTACTACTGTAYG; | (SEQ ID NO:56) |
| CCACACTTTGCCCRT; | (SEQ ID NO:57) |
| CTGGAAAGCCTACYA; | (SEQ ID NO:58) |
| TCATTGCAAGTCTRG; | (SEQ ID NO:59) |
| TGTTAAATGGTGAYA; | (SEQ ID NO:60) |
| AGCAGGTATTACTRT; | (SEQ ID NO:61) |
| TCAGACTTCTGAAMC; | (SEQ ID NO:62) |
| GCTTAGGATGTGGKT; | (SEQ ID NO:63) |
| CATCAGCAATTGCYA; | (SEQ ID NO:64) |
| TTGACAATTGAGTRG; | (SEQ ID NO:65) |
| AAACACAGCTTGCRA; | (SEQ ID NO:66) |
| TTTCTATGTATATYG; | (SEQ ID NO:67) |
| ACTGAGTGAAACTRG; | (SEQ ID NO:68) |
| and | |
| CATGCCACTTAACYA. | (SEQ ID NO:69) |

Other oligonucleotides of the invention hybridize to a target region located one to several nucleotides downstream of one of the novel polymorphic sites identified herein. Such oligonucleotides are useful in polymerase-mediated primer extension methods for detecting one of the novel polymorphisms described herein and therefore such oligonucleotides are referred to herein as “primer-extension oligonucleotides”. In a preferred embodiment, the 3′-terminus of a primer-extension oligonucleotide is a deoxynucleotide complementary to the nucleotide located immediately adjacent to the polymorphic site.

A particularly preferred oligonucleotide primer for detecting FCER1A gene polymorphisms by primer extension terminates in a nucleotide sequence, listed 5′ to 3′, selected from the group consisting of:

| | |
|---|---|
| AAATGAAATA; | (SEQ ID NO:70) |
| AATAAATCTG; | (SEQ ID NO:71) |
| TTTATTCTGC; | (SEQ ID NO:72) |
| TGCAAGGGAG; | (SEQ ID NO:73) |
| CCAGATATGA; | (SEQ ID NO:74) |
| TGTTTTCTGT; | (SEQ ID NO:75) |
| TGATACAGAA; | (SEQ ID NO:76) |
| AAGGAAATGT; | (SEQ ID NO:77) |
| TTCAATTACC; | (SEQ ID NO:78) |
| TCCCTGGGAG; | (SEQ ID NO:79) |
| GACACTAATG; | (SEQ ID NO:80) |
| CAGAGAGGAT; | (SEQ ID NO:81) |
| AATGTATCCT; | (SEQ ID NO:82) |
| AAAGTCCAGA; | (SEQ ID NO:83) |
| TTCTAATGAG; | (SEQ ID NO:84) |
| ACAGATTCAT; | (SEQ ID NO:85) |
| AGAAATCAAA; | (SEQ ID NO:86) |
| TAAGACCCTG; | (SEQ ID NO:87) |
| GTGAATGCCA; | (SEQ ID NO:88) |
| GTCTTCAAAT; | (SEQ ID NO:89) |
| GTTAATGAGA; | (SEQ ID NO:90) |
| CACAGGTTCA; | (SEQ ID NO:91) |
| TCAAGGCCTC; | (SEQ ID NO:92) |
| CTTAAAAATG; | (SEQ ID NO:93) |
| CACTTTGGGA; | (SEQ ID NO:94) |
| CTGCCTCAGC; | (SEQ ID NO:95) |
| GAGACCATCC; | (SEQ ID NO:96) |
| CCATGTTAGC; | (SEQ ID NO:97) |
| TCTATGCGTG; | (SEQ ID NO:98) |
| GAAAAGAGAG; | (SEQ ID NO:99) |
| TACTACTGTA; | (SEQ ID NO:100) |
| CACTTTGCCC; | (SEQ ID NO:101) |
| GAAAGCCTAC; | (SEQ ID NO:102) |
| TTGCAAGTCT; | (SEQ ID NO:103) |
| TAAATGGTGA; | (SEQ ID NO:104) |
| AGGTATTACT; | (SEQ ID NO:105) |
| GACTTCTGAA; | (SEQ ID NO:106) |
| TAGGATGTGG; | (SEQ ID NO:107) |
| CAGCAATTGC; | (SEQ ID NO:108) |
| ACAATTGAGT; | (SEQ ID NO:109) |
| CACAGCTTGC; | (SEQ ID NO:110) |
| CTATGTATAT; | (SEQ ID NO:111) |
| GAGTGAAACT; | (SEQ ID NO:112) |
| and | |
| GCCACTTAAC. | (SEQ ID NO:113) |

In some embodiments, a composition contains two or more differently labeled FCER1A oligonucleotides for simultaneously probing the identity of nucleotides or nucleotide pairs at two or more polymorphic sites. It is also contemplated that primer compositions may contain two or more sets of allele-specific primer pairs to allow simultaneous targeting and amplification of two or more regions containing a polymorphic site.

FCER1A oligonucleotides of the invention may also be immobilized on or synthesized on a solid surface such as a microchip, bead, or glass slide (see, e.g., WO 98/20020 and WO 98/20019). Such immobilized oligonucleotides may be used in a variety of polymorphism detection assays, including but not limited to probe hybridization and polymerase extension assays. Immobilized FCER1A oligonucleotides of the invention may comprise an ordered array of oligonucleotides designed to rapidly screen a DNA sample for polymorphisms in multiple genes at the same time.

In another embodiment, the invention provides a kit comprising at least two FCER1A oligonucleotides packaged in separate containers. The kit may also contain other components such as hybridization buffer (where the oligonucleotides are to be used as a probe) packaged in a separate container. Alternatively, where the oligonucleotides are to be used to amplify a target region, the kit may contain, packaged in separate containers, a polymerase and a reaction buffer optimized for primer extension mediated by the polymerase, such as PCR.

The above described oligonucleotide compositions and kits are useful in methods for genotyping and/or haplotyping the FCER1A gene in an individual. As used herein, the terms “FCER1A genotype” and “FCER1A haplotype” mean the genotype or haplotype contains the nucleotide pair or nucleotide, respectively, that is present at one or more of the novel polymorphic sites described herein and may optionally also include the nucleotide pair or nucleotide present at one or more additional polymorphic sites in the FCER1A gene. The additional polymorphic sites may be currently known polymorphic sites or sites that are subsequently discovered.

One embodiment of a genotyping method of the invention involves examining both copies of the individual's FCER1A gene, or a fragment thereof, to identify the nucleotide pair at one or more polymorphic sites selected from the group consisting of PS1, PS2, PS3, PS4, PS5, PS6, PS7, PS8, PS9, PS10, PS11, PS12, PS13, PS14, PS15, PS16, PS17, PS18, PS19, PS20, PS21 and PS22 in the two copies to assign a FCER1A genotype to the individual. In some embodiments, “examining a gene” may include examining one or more of: DNA containing the gene, mRNA transcripts thereof, or cDNA copies thereof. As will be readily understood by the skilled artisan, the two “copies” of a gene, mRNA or cDNA (or fragment of such FCER1A molecules) in an individual may be the same allele or may be different alleles. In a preferred embodiment of the method for assigning a FCER1A genotype, the identity of the nucleotide pair at PS4 is also determined. In another embodiment, a genotyping method of the invention comprises determining the identity of the nucleotide pair at each of PS1-PS22.

One method of examining both copies of the individual's FCER1A gene is by isolating from the individual a nucleic acid sample comprising the two copies of the FCER1A gene, mRNA transcripts thereof or cDNA copies thereof, or a fragment of any of the foregoing, that are present in the individual. Typically, the nucleic acid sample is isolated from a biological sample taken from the individual, such as a blood sample or tissue sample. Suitable tissue samples include whole blood, semen, saliva, tears, urine, fecal material, sweat, buccal, skin and hair. The nucleic acid sample may be comprised of genomic DNA, mRNA, or cDNA and, in the latter two cases, the biological sample must be obtained from a tissue in which the FCER1A gene is expressed. Furthermore it will be understood by the skilled artisan that mRNA or cDNA preparations would not be used to detect polymorphisms located in introns or in 5′ and 3′ untranslated regions if not present in the mRNA or cDNA. If a FCER1A gene fragment is isolated, it must contain the polymorphic site(s) to be genotyped.

One embodiment of a haplotyping method of the invention comprises examining one copy of the individual's FCER1A gene, or a fragment thereof, to identify the nucleotide at one or more polymorphic sites selected from the group consisting of PS1, PS2, PS3, PS4, PS5, PS6, PS7, PS8, PS9, PS10, PS11, PS12, PS13, PS14, PS15, PS16, PS17, PS18, PS19, PS20, PS21 and PS22 in that copy to assign a FCER1A haplotype to the individual. In some embodiments, “examining a gene” may include examining one or more of: DNA containing the gene, mRNA transcripts thereof, or cDNA copies thereof. One method of examining one copy of the individual's DNA is by isolating from the individual a nucleic acid sample containing only one of the two copies of the FCER1A gene, mRNA or cDNA, or a fragment of such FCER1A molecules, that is present in the individual and determining in that copy the identity of the nucleotide at one or more polymorphic sites selected from the group consisting of PS1, PS2, PS3, PS4, PS5, PS6, PS7, PS8, PS9, PS10, PS11, PS12, PS13, PS14, PS15, PS16, PS17, PS18, PS19, PS20, PS21 and PS22 in that copy to assign a FCER1A haplotype to the individual.

The nucleic acid used in the above haplotyping methods of the invention may be isolated using any method capable of separating the two copies of the FCER1A gene or fragment such as one of the methods described above for preparing FCER1A isogenes, with targeted in vivo cloning being the preferred approach. As will be readily appreciated by those skilled in the art, any individual clone will typically only provide haplotype information on one of the two FCER1A gene copies present in an individual. If haplotype information is desired for the individual's other copy, additional FCER1A clones will usually need to be examined. Typically, at least five clones should be examined to have more than a 90% probability of haplotyping both copies of the FCER1A gene in an individual. In some cases, however, once the haplotype for one FCER1A allele is directly determined, the haplotype for the other allele may be inferred if the individual has a known genotype for the polymorphic sites of interest or if the haplotype frequency or haplotype pair frequency for the individual's population group is known. In a particularly preferred embodiment, the nucleotide at each of PS1-PS22 is identified.

In another embodiment, the haplotyping method comprises determining whether an individual has one or more of the FCER1A haplotypes shown in Table 5. This can be accomplished by identifying, for one or both copies of the individual's FCER1A gene, the phased sequence of nucleotides present at each of PS1-PS22. This identifying step does not necessarily require that each of PS1-PS22 be directly examined. Typically only a subset of PS1-PS22 will need to be directly examined to assign to an individual one or more of the haplotypes shown in Table 5. This is because at least one polymorphic site in a gene is frequently in strong linkage disequilibrium with one or more other polymorphic sites in that gene (Drysdale, C M et al. 2000 PNAS 97:10483-10488; Rieder M J et al. 1999 Nature Genetics 22:59-62). Two sites are said to be in linkage disequilibrium if the presence of a particular variant at one site enhances the predictability of another variant at the second site (Stephens, J C 1999, Mol. Diag. 4:309-317). Techniques for determining whether any two polymorphic sites are in linkage disequilibrium are well-known in the art (Weir B. S. 1996 Genetic Data Analysis II, Sinauer Associates, Inc. Publishers, Sunderland, Mass.). In addition, Johnson et al. (2001 Nature Genetics 29: 233-237) presented one possible method for selection of subsets of polymorphic sites suitable for identifying known haplotypes.

In another embodiment of a haplotyping method of the invention, a FCER1A haplotype pair is determined for an individual by identifying the phased sequence of nucleotides at one or more polymorphic sites selected from the group consisting of PS1, PS2, PS3, PS4, PS5, PS6, PS7, PS8, PS9, PS10, PS11, PS12, PS13, PS14, PS15, PS16, PS17, PS18, PS19, PS20, PS21 and PS22 in each copy of the FCER1A gene that is present in the individual. In a particularly preferred embodiment, the haplotyping method comprises identifying the phased sequence of nucleotides at each of PS1-PS22 in each copy of the FCER1A gene.

When haplotyping both copies of the gene, the identifying step is preferably performed with each copy of the gene being placed in separate containers. However, it is also envisioned that if the two copies are labeled with different tags, or are otherwise separately distinguishable or identifiable, it could be possible in some cases to perform the method in the same container. For example, if first and second copies of the gene are labeled with different first and second fluorescent dyes, respectively, and an allele-specific oligonucleotide labeled with yet a third different fluorescent dye is used to assay the polymorphic site(s), then detecting a combination of the first and third dyes would identify the polymorphism in the first gene copy while detecting a combination of the second and third dyes would identify the polymorphism in the second gene copy.

In both the genotyping and haplotyping methods, the identity of a nucleotide (or nucleotide pair) at a polymorphic site(s) may be determined by amplifying a target region(s) containing the polymorphic site(s) directly from one or both copies of the FCER1A gene, or a fragment thereof, and the sequence of the amplified region(s) determined by conventional methods. It will be readily appreciated by the skilled artisan that only one nucleotide will be detected at a polymorphic site in individuals who are homozygous at that site, while two different nucleotides will be detected if the individual is heterozygous for that site. The polymorphism may be identified directly, known as positive-type identification, or by inference, referred to as negative-type identification. For example, where a SNP is known to be guanine and cytosine in a reference population, a site may be positively determined to be either guanine or cytosine for an individual homozygous at that site, or both guanine and cytosine, if the individual is heterozygous at that site. Alternatively, the site may be negatively determined to be not guanine (and thus cytosine/cytosine) or not cytosine (and thus guanine/guanine).

The target region(s) may be amplified using any oligonucleotide-directed amplification method, including but not limited to polymerase chain reaction (PCR) (U.S. Pat. No. 4,965,188), ligase chain reaction (LCR) (Barany et al., Proc. Natl. Acad. Sci. USA 88:189-193, 1991; WO90/01069), and oligonucleotide ligation assay (OLA) (Landegren et al., Science 241:1077-1080, 1988). Other known nucleic acid amplification procedures may be used to amplify the target region including transcription-based amplification systems (U.S. Pat. No. 5,130,238; EP 329,822; U.S. Pat. No. 5,169,766, WO89/06700) and isothermal methods (Walker et al., Proc. Natl. Acad. Sci. USA 89:392-396, 1992).

A polymorphism in the target region may also be assayed before or after amplification using one of several hybridization-based methods known in the art. Typically, allele-specific oligonucleotides are utilized in performing such methods. The allele-specific oligonucleotides may be used as differently labeled probe pairs, with one member of the pair showing a perfect match to one variant of a target sequence and the other member showing a perfect match to a different variant. In some embodiments, more than one polymorphic site may be detected at once using a set of allele-specific oligonucleotides or oligonucleotide pairs. Preferably, the members of the set have melting temperatures within 5° C., and more preferably within 2° C., of each other when hybridizing to each of the polymorphic sites being detected.

Hybridization of an allele-specific oligonucleotide to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Allele-specific oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the allele-specific oligonucleotide or target nucleic acid.

The genotype or haplotype for the FCER1A gene of an individual may also be determined by hybridization of a nucleic acid sample containing one or both copies of the gene, mRNA, cDNA or fragment(s) thereof, to nucleic acid arrays and subarrays such as described in WO 95/11995. The arrays would contain a battery of allele-specific oligonucleotides representing each of the polymorphic sites to be included in the genotype or haplotype.

The identity of polymorphisms may also be determined using a mismatch detection technique, including but not limited to the RNase protection method using riboprobes (Winter et al., Proc. Natl. Acad. Sci. USA 82:7575, 1985; Meyers et al., Science 230:1242, 1985) and proteins which recognize nucleotide mismatches, such as the E. coli mutS protein (Modrich, P. Ann. Rev. Genet. 25:229-253, 1991). Alternatively, variant alleles can be identified by single strand conformation polymorphism (SSCP) analysis (Orita et al., Genomics 5:874-879, 1989; Humphries et al., in Molecular Diagnosis of Genetic Diseases, R. Elles, ed., pp. 321-340, 1996) or denaturing gradient gel electrophoresis (DGGE) (Wartell et al., Nucl. Acids Res. 18:2699-2706, 1990; Sheffield et al., Proc. Natl. Acad. Sci. USA 86:232-236, 1989).

A polymerase-mediated primer extension method may also be used to identify the polymorphism(s). Several such methods have been described in the patent and scientific literature and include the “Genetic Bit Analysis” method (WO92/15712) and the ligase/polymerase mediated genetic bit analysis (U.S. Pat. No. 5,679,524. Related methods are disclosed in WO91/02087, WO90/09455, WO95/17676, U.S. Pat. Nos. 5,302,509, and 5,945,283. Extended primers containing a polymorphism may be detected by mass spectrometry as described in U.S. Pat. No. 5,605,798. Another primer extension method is allele-specific PCR (Ruaño et al., Nucl. Acids Res. 17:8392, 1989; Ruaño et al., Nucl. Acids Res. 19, 6877-6882, 1991; WO 93/22456; Turki et al., J. Clin. Invest. 95:1635-1641, 1995). In addition, multiple polymorphic sites may be investigated by simultaneously amplifying multiple regions of the nucleic acid using sets of allele-specific primers as described in Wallace et al. (WO89/10414).

In addition, the identity of the allele(s) present at any of the novel polymorphic sites described herein may be indirectly determined by haplotyping or genotyping another polymorphic site that is in linkage disequilibrium with the polymorphic site that is of interest. Polymorphic sites in linkage disequilibrium with the presently disclosed polymorphic sites may be located in regions of the gene or in other genomic regions not examined herein. Detection of the allele(s) present at a polymorphic site in linkage disequilibrium with the novel polymorphic sites described herein may be performed by, but is not limited to, any of the above-mentioned methods for detecting the identity of the allele at a polymorphic site.

In another aspect of the invention, an individual's FCER1A haplotype pair is predicted from its FCER1A genotype using information on haplotype pairs known to exist in a reference population. In its broadest embodiment, the haplotyping prediction method comprises identifying a FCER1A genotype for the individual at two or more FCER1A polymorphic sites described herein, accessing data containing FCER1A haplotype pairs identified in a reference population, and assigning a haplotype pair to the individual that is consistent with the genotype data. In one embodiment, the reference haplotype pairs include the FCER1A haplotype pairs shown in Table 4. The FCER1A haplotype pair can be assigned by comparing the individual's genotype with the genotypes corresponding to the haplotype pairs known to exist in the general population or in a specific population group, and determining which haplotype pair is consistent with the genotype of the individual. In some embodiments, the comparing step may be performed by visual inspection (for example, by consulting Table 4). When the genotype of the individual is consistent with more than one haplotype pair, frequency data (such as that presented in Table 7) may be used to determine which of these haplotype pairs is most likely to be present in the individual. This determination may also be performed in some embodiments by visual inspection, for example by consulting Table 7. If a particular FCER1A haplotype pair consistent with the genotype of the individual is more frequent in the reference population than others consistent with the genotype, then that haplotype pair with the highest frequency is the most likely to be present in the individual. In other embodiments, the comparison may be made by a computer-implemented algorithm with the genotype of the individual and the reference haplotype data stored in computer-readable formats. For example, as described in PCT/US01/12831, filed Apr. 18, 2001, one computer-implemented algorithm to perform this comparison entails enumerating all possible haplotype pairs which are consistent with the genotype, accessing data containing FCER1A haplotype pairs frequency data determined in a reference population to determine a probability that the individual has a possible haplotype pair, and analyzing the determined probabilities to assign a haplotype pair to the individual.

Generally, the reference population should be composed of randomly-selected individuals representing the major ethnogeographic groups of the world. A preferred reference population for use in the methods of the present invention comprises an approximately equal number of individuals from Caucasian, African-descent, Asian and Hispanic-Latino population groups with the minimum number of each group being chosen based on how rare a haplotype one wants to be guaranteed to see. For example, if one wants to have a q % chance of not missing a haplotype that exists in the population at a p % frequency of occurring in the reference population, the number of individuals (n) who must be sampled is given by 2n=log(1−q)/log(1−p) where p and q are expressed as fractions. A preferred reference population allows the detection of any haplotype whose frequency is at least 10% with about 99% certainty and comprises about 20 unrelated individuals from each of the four population groups named above. A particularly preferred reference population includes a 3-generation family representing one or more of the four population groups to serve as controls for checking quality of haplotyping procedures.

In a preferred embodiment, the haplotype frequency data for each ethnogeographic group is examined to determine whether it is consistent with Hardy-Weinberg equilibrium. Hardy-Weinberg equilibrium (D. L. Hartl et al., Principles of Population Genomics, Sinauer Associates (Sunderland, MA), 3^{rd} Ed., 1997) postulates that the frequency of finding the haplotype pair H₁/H₂ is equal to p_{H-W}(H₁/H₂)=2p(H₁)p(H₂) if H₁≠H₂ and p_{H-W}(H₁/H₂)=p(H₁)p(H₂) if H₁=H₂. A statistically significant difference between the observed and expected haplotype frequencies could be due to one or more factors including significant inbreeding in the population group, strong selective pressure on the gene, sampling bias, and/or errors in the genotyping process. If large deviations from Hardy-Weinberg equilibrium are observed in an ethnogeographic group, the number of individuals in that group can be increased to see if the deviation is due to a sampling bias. If a larger sample size does not reduce the difference between observed and expected haplotype pair frequencies, then one may wish to consider haplotyping the individual using a direct haplotyping method such as, for example, CLASPER System™ technology (U.S. Pat. No. 5,866,404), single molecule dilution, or allele-specific long-range PCR (Michalotos-Beloin et al., Nucleic Acids Res. 24:4841-4843, 1996).

In one embodiment of this method for predicting a FCER1A haplotype pair for an individual, the assigning step involves performing the following analysis. First, each of the possible haplotype pairs is compared to the haplotype pairs in the reference population. Generally, only one of the haplotype pairs in the reference population matches a possible haplotype pair and that pair is assigned to the individual. Occasionally, only one haplotype represented in the reference haplotype pairs is consistent with a possible haplotype pair for an individual, and in such cases the individual is assigned a haplotype pair containing this known haplotype and a new haplotype derived by subtracting the known haplotype from the possible haplotype pair. Alternatively, the haplotype pair in an individual may be predicted from the individual's genotype for that gene using reported methods (e.g., Clark et al. 1990 Mol Bio Evol 7:111-22; copending PCT/US01/12831 filed Apr. 18, 2001) or through a commercial haplotyping service such as offered by Genaissance Pharmaceuticals, Inc. (New Haven, Conn.). In rare cases, either no haplotypes in the reference population are consistent with the possible haplotype pairs, or alternatively, multiple reference haplotype pairs are consistent with the possible haplotype pairs. In such cases, the individual is preferably haplotyped using a direct molecular haplotyping method such as, for example, CLASPER System™ technology (U.S. Pat. No. 5,866,404), SMD, or allele-specific long-range PCR (Michalotos-Beloin et al., supra).

The invention also provides a method for determining the frequency of a FCER1A genotype, haplotype, or haplotype pair in a population. The method comprises, for each member of the population, determining the genotype or the haplotype pair for the novel FCER1A polymorphic sites described herein, and calculating the frequency any particular genotype, haplotype, or haplotype pair is found in the population. The population may be e.g., a reference population, a family population, a same gender population, a population group, or a trait population (e.g., a group of individuals exhibiting a trait of interest such as a medical condition or response to a therapeutic treatment).

In another aspect of the invention, frequency data for FCER1A genotypes, haplotypes, and/or haplotype pairs are determined in a reference population and used in a method for identifying an association between a trait and a FCER1A genotype, haplotype, or haplotype pair. The trait may be any detectable phenotype, including but not limited to susceptibility to a disease or response to a treatment. In one embodiment, the method involves obtaining data on the frequency of the genotype(s), haplotype(s), or haplotype pair(s) of interest in a reference population as well as in a population exhibiting the trait. Frequency data for one or both of the reference and trait populations may be obtained by genotyping or haplotyping each individual in the populations using one or more of the methods described above. The haplotypes for the trait population may be determined directly or, alternatively, by a predictive genotype to haplotype approach as described above. In another embodiment, the frequency data for the reference and/or trait populations is obtained by accessing previously determined frequency data, which may be in written or electronic form. For example, the frequency data may be present in a database that is accessible by a computer. Once the frequency data is obtained, the frequencies of the genotype(s), haplotype(s), or haplotype pair(s) of interest in the reference and trait populations are compared. In a preferred embodiment, the frequencies of all genotypes, haplotypes, and/or haplotype pairs observed in the populations are compared. If a particular FCER1A genotype, haplotype, or haplotype pair is more frequent in the trait population than in the reference population at a statistically significant amount, then the trait is predicted to be associated with that FCER1A genotype, haplotype or haplotype pair. Preferably, the FCER1A genotype, haplotype, or haplotype pair being compared in the trait and reference populations is selected from the full-genotypes and full-haplotypes shown in Tables 4 and 5, or from sub-genotypes and sub-haplotypes derived from these genotypes and haplotypes.

In a preferred embodiment of the method, the trait of interest is a clinical response exhibited by a patient to some therapeutic treatment, for example, response to a drug targeting FCER1A or response to a therapeutic treatment for a medical condition. As used herein, “medical condition” includes but is not limited to any condition or disease manifested as one or more physical and/or psychological symptoms for which treatment is desirable, and includes previously and newly identified diseases and other disorders. As used herein the term “clinical response” means any or all of the following: a quantitative measure of the response, no response, and/or adverse response (i.e., side effects).

In order to deduce a correlation between clinical response to a treatment and a FCER1A genotype, haplotype, or haplotype pair, it is necessary to obtain data on the clinical responses exhibited by a population of individuals who received the treatment, hereinafter the “clinical population”. This clinical data may be obtained by analyzing the results of a clinical trial that has already been run and/or the clinical data may be obtained by designing and carrying out one or more new clinical trials. As used herein, the term “clinical trial” means any research study designed to collect clinical data on responses to a particular treatment, and includes but is not limited to phase I, phase II and phase III clinical trials. Standard methods are used to define the patient population and to enroll subjects.

It is preferred that the individuals included in the clinical population have been graded for the existence of the medical condition of interest. This is important in cases where the symptom(s) being presented by the patients can be caused by more than one underlying condition, and where treatment of the underlying conditions are not the same. An example of this would be where patients experience breathing difficulties that are due to either asthma or respiratory infections. If both sets were treated with an asthma medication, there would be a spurious group of apparent non-responders that did not actually have asthma. These people would affect the ability to detect any correlation between haplotype and treatment outcome. This grading of potential patients could employ a standard physical exam or one or more lab tests. Alternatively, grading of patients could use haplotyping for situations where there is a strong correlation between haplotype pair and disease susceptibility or severity.

The therapeutic treatment of interest is administered to each individual in the trial population and each individual's response to the treatment is measured using one or more predetermined criteria. It is contemplated that in many cases, the trial population will exhibit a range of responses and that the investigator will choose the number of responder groups (e.g., low, medium, high) made up by the various responses. In addition, the FCER1A gene for each individual in the trial population is genotyped and/or haplotyped, which may be done before or after administering the treatment.

After both the clinical and polymorphism data have been obtained, correlations between individual response and FCER1A genotype or haplotype content are created. Correlations may be produced in several ways. In one method, individuals are grouped by their FCER1A genotype or haplotype (or haplotype pair) (also referred to as a polymorphism group), and then the averages and standard deviations of clinical responses exhibited by the members of each polymorphism group are calculated.

These results are then analyzed to determine if any observed variation in clinical response between polymorphism groups is statistically significant. Statistical analysis methods which may be used are described in L. D. Fisher and G. vanBelle, “Biostatistics: A Methodology for the Health Sciences”, Wiley-Interscience (New York) 1993. This analysis may also include a regression calculation of which polymorphic sites in the FCER1A gene give the most significant contribution to the differences in phenotype. One regression model useful in the invention is described in WO 01/01218, entitled “Methods for Obtaining and Using Haplotype Data”.

A second method for finding correlations between FCER1A haplotype content and clinical responses uses predictive models based on error-minimizing optimization algorithms. One of many possible optimization algorithms is a genetic algorithm (R. Judson, “Genetic Algorithms and Their Uses in Chemistry” in Reviews in Computational Chemistry, Vol. 10, pp. 1-73, K. B. Lipkowitz and D. B. Boyd, eds. (VCH Publishers, New York, 1997). Simulated annealing (Press et al., “Numerical Recipes in C: The Art of Scientific Computing”, Cambridge University Press (Cambridge) 1992, Ch. 10), neural networks (E. Rich and K. Knight, “Artificial Intelligence”, 2^{nd} Edition (McGraw-Hill, New York, 1991, Ch. 18), standard gradient descent methods (Press et al., supra, Ch. 10), or other global or local optimization approaches (see discussion in Judson, supra) could also be used. Preferably, the correlation is found using a genetic algorithm approach as described in WO 01/01218.

Correlations may also be analyzed using analysis of variation (ANOVA) techniques to determine how much of the variation in the clinical data is explained by different subsets of the polymorphic sites in the FCER1A gene. As described in WO 01/01218, ANOVA is used to test hypotheses about whether a response variable is caused by or correlated with one or more traits or variables that can be measured (Fisher and vanBelle, supra, Ch. 10).

From the analyses described above, a mathematical model may be readily constructed by the skilled artisan that predicts clinical response as a function of FCER1A genotype or haplotype content. Preferably, the model is validated in one or more follow-up clinical trials designed to test the model.

The identification of an association between a clinical response and a genotype or haplotype (or haplotype pair) for the FCER1A gene may be the basis for designing a diagnostic method to determine those individuals who will or will not respond to the treatment, or alternatively, will respond at a lower level and thus may require more treatment, i.e., a greater dose of a drug. The diagnostic method may take one of several forms: for example, a direct DNA test (i.e., genotyping or haplotyping one or more of the polymorphic sites in the FCER1A gene), a serological test, or a physical exam measurement. The only requirement is that there be a good correlation between the diagnostic test results and the underlying FCER1A genotype or haplotype that is in turn correlated with the clinical response. In a preferred embodiment, this diagnostic method uses the predictive haplotyping method described above.

In another embodiment, the invention provides an isolated polynucleotide comprising a polymorphic variant of the FCER1A gene or a fragment of the gene which contains at least one of the novel polymorphic sites described herein. The nucleotide sequence of a variant FCER1A gene is identical to the reference genomic sequence for those portions of the gene examined, as described in the Examples below, except that it comprises a different nucleotide at one or more of the novel polymorphic sites PS1, PS2, PS3, PS4, PS5, PS6, PS7, PS8, PS9, PS10, PS11, PS12, PS13, PS14, PS15, PS16, PS17, PS18, PS19, PS20, PS21 and PS22. Similarly, the nucleotide sequence of a variant fragment of the FCER1A gene is identical to the corresponding portion of the reference sequence except for having a different nucleotide at one or more of the novel polymorphic sites described herein. Thus, the invention specifically does not include polynucleotides comprising a nucleotide sequence identical to the reference sequence of the FCER1A gene, which is defined by haplotype 2, (or other reported FCER1A sequences) or to portions of the reference sequence (or other reported FCER1A sequences), except for the haplotyping and genotyping oligonucleotides described above.

The location of a polymorphism in a variant FCER1A gene or fragment is preferably identified by aligning its sequence against SEQ ID NO:1. The polymorphism is selected from the group consisting of guanine at PS1, cytosine at PS2, cytosine at PS3, thymine at PS4, adenine at PS5, cytosine at PS6, adenine at PS7, thymine at PS8, guanine at PS9, guanine at PS10, adenine at PS11, cytosine at PS12, adenine at PS13, adenine at PS14, adenine at PS15, thymine at PS16, thymine at PS17, cytosine at PS18, adenine at PS19, cytosine at PS20, guanine at PS21 and adenine at PS22. In a preferred embodiment, the polymorphic variant comprises a naturally-occurring isogene of the FCER1A gene which is defined by any one of haplotypes 1 and 3-20 shown in Table 5 below.

Polymorphic variants of the invention may be prepared by isolating a clone containing the FCER1A gene from a human genomic library. The clone may be sequenced to determine the identity of the nucleotides at the novel polymorphic sites described herein. Any particular variant or fragment thereof, that is claimed herein could be prepared from this clone by performing in vitro mutagenesis using procedures well-known in the art. Any particular FCER1A variant or fragment thereof may also be prepared using synthetic or semi-synthetic methods known in the art.

FCER1A isogenes, or fragments thereof, may be isolated using any method that allows separation of the two “copies” of the FCER1A gene present in an individual, which, as readily understood by the skilled artisan, may be the same allele or different alleles. Separation methods include targeted in vivo cloning (TIVC) in yeast as described in WO 98/01573, U.S. Pat. No. 5,866,404, and U.S. Pat. No. 5,972,614. Another method, which is described in U.S. Pat. No. 5,972,614, uses an allele specific oligonucleotide in combination with primer extension and exonuclease degradation to generate hemizygous DNA targets. Yet other methods are single molecule dilution (SMD) as described in Ruaño et al., Proc. Natl. Acad. Sci. 87:6296-6300, 1990; and allele specific PCR (Ruaño et al., 1989, supra; Ruaño et al., 1991, supra; Michalatos-Beloin et al., supra).

The invention also provides FCER1A genome anthologies, which are collections of at least two FCER1A isogenes found in a given population. The population may be any group of at least two individuals, including but not limited to a reference population, a population group, a family population, a clinical population, and a same gender population. A FCER1A genome anthology may comprise individual FCER1A isogenes stored in separate containers such as microtest tubes, separate wells of a microtitre plate and the like. Alternatively, two or more groups of the FCER1A isogenes in the anthology may be stored in separate containers. Individual isogenes or groups of such isogenes in a genome anthology may be stored in any convenient and stable form, including but not limited to in buffered solutions, as DNA precipitates, freeze-dried preparations and the like. A preferred FCER1A genome anthology of the invention comprises a set of isogenes defined by the haplotypes shown in Table 5 below.

An isolated polynucleotide containing a polymorphic variant nucleotide sequence of the invention may be operably linked to one or more expression regulatory elements in a recombinant expression vector capable of being propagated and expressing the encoded FCER1A protein in a prokaryotic or a eukaryotic host cell. Examples of expression regulatory elements which may be used include, but are not limited to, the lac system, operator and promoter regions of phage lambda, yeast promoters, and promoters derived from vaccinia virus, adenovirus, retroviruses, or SV40. Other regulatory elements include, but are not limited to, appropriate leader sequences, termination codons, polyadenylation signals, and other sequences required for the appropriate transcription and subsequent translation of the nucleic acid sequence in a given host cell. Of course, the correct combinations of expression regulatory elements will depend on the host system used. In addition, it is understood that the expression vector contains any additional elements necessary for its transfer to and subsequent replication in the host cell. Examples of such elements include, but are not limited to, origins of replication and selectable markers. Such expression vectors are commercially available or are readily constructed using methods known to those in the art (e.g., F. Ausubel et al., 1987, in “Current Protocols in Molecular Biology”, John Wiley and Sons, New York, N.Y.). Host cells which may be used to express the variant FCER1A sequences of the invention include, but are not limited to, eukaryotic and mammalian cells, such as animal, plant, insect and yeast cells, and prokaryotic cells, such as E. coli, or algal cells as known in the art. The recombinant expression vector may be introduced into the host cell using any method known to those in the art including, but not limited to, microinjection, electroporation, particle bombardment, transduction, and transfection using DEAE-dextran, lipofection, or calcium phosphate (see e.g., Sambrook et al. (1989) in “Molecular Cloning. A Laboratory Manual”, Cold Spring Harbor Press, Plainview, N.Y.). In a preferred aspect, eukaryotic expression vectors that function in eukaryotic cells, and preferably mammalian cells, are used. Non-limiting examples of such vectors include vaccinia virus vectors, adenovirus vectors, herpes virus vectors, and baculovirus transfer vectors. Preferred eukaryotic cell lines include COS cells, CHO cells, HeLa cells, NIH/3T3 cells, and embryonic stem cells (Thomson, J. A. et al., 1998 Science 282:1145-1147). Particularly preferred host cells are mammalian cells.

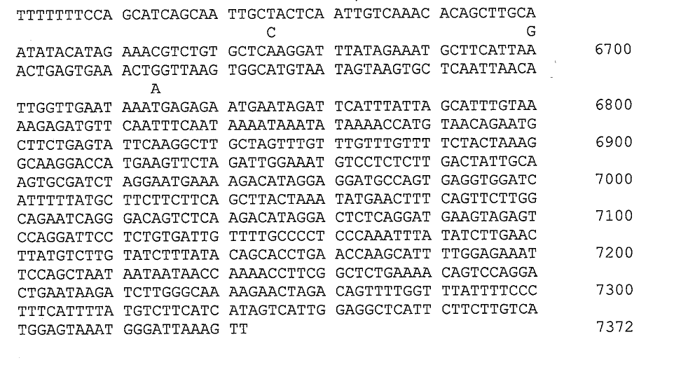

As will be readily recognized by the skilled artisan, expression of polymorphic variants of the FCER1A gene will produce FCER1A mRNAs varying from each other at any polymorphic site retained in the spliced and processed mRNA molecules. These mRNAs can be used for the preparation of a FCER1A cDNA comprising a nucleotide sequence which is a polymorphic variant of the FCER1A reference coding sequence shown in FIG. 2. Thus, the invention also provides FCER1A mRNAs and corresponding cDNAs which comprise a nucleotide sequence that is identical to SEQ ID NO:2 (FIG. 2) (or its corresponding RNA sequence) for those regions of SEQ ID NO:2 that correspond to the examined portions of the FCER1A gene (as described in the Examples below), except for having one or more polymorphisms selected from the group consisting of guanine at a position corresponding to nucleotide 251, adenine at a position corresponding to nucleotide 302, thymine at a position corresponding to nucleotide 530 and adenine at a position corresponding to nucleotide 741. A particularly preferred polymorphic cDNA variant comprises the coding sequence of a FCER1A isogene defined by any one of haplotypes 7, 10, 12, 16, 17, and 19. Fragments of these variant mRNAs and cDNAs are included in the scope of the invention, provided they contain one or more of the novel polymorphisms described herein. The invention specifically excludes polynucleotides identical to previously identified FCER1A mRNAs or cDNAs, and previously described fragments thereof. Polynucleotides comprising a variant FCER1A RNA or DNA sequence may be isolated from a biological sample using well-known molecular biological procedures or may be chemically synthesized.

As used herein, a polymorphic variant of a FCER1A gene, mRNA or cDNA fragment comprises at least one novel polymorphism identified herein and has a length of at least 10 nucleotides and may range up to the full length of the gene. Preferably, such fragments are between 100 and 3000 nucleotides in length, and more preferably between 200 and 2000 nucleotides in length, and most preferably between 500 and 1000 nucleotides in length.

In describing the FCER1A polymorphic sites identified herein, reference is made to the sense strand of the gene for convenience. However, as recognized by the skilled artisan, nucleic acid molecules containing the FCER1A gene or cDNA may be complementary double stranded molecules and thus reference to a particular site on the sense strand refers as well to the corresponding site on the complementary antisense strand. Thus, reference may be made to the same polymorphic site on either strand and an oligonucleotide may be designed to hybridize specifically to either strand at a target region containing the polymorphic site. Thus, the invention also includes single-stranded polynucleotides which are complementary to the sense strand of the FCER1A genomic, mRNA and cDNA variants described herein.

Polynucleotides comprising a polymorphic gene variant or fragment of the invention may be useful for therapeutic purposes. For example, where a patient could benefit from expression, or increased expression, of a particular FCER1A protein isoform, an expression vector encoding the isoform may be administered to the patient. The patient may be one who lacks the FCER1A isogene encoding that isoform or may already have at least one copy of that isogene.

In other situations, it may be desirable to decrease or block expression of a particular FCER1A isogene. Expression of a FCER1A isogene may be turned off by transforming a targeted organ, tissue or cell population with an expression vector that expresses high levels of untranslatable mRNA or antisense RNA for the isogene or fragment thereof. Alternatively, oligonucleotides directed against the regulatory regions (e.g., promoter, introns, enhancers, 3′ untranslated region) of the isogene may block transcription. Oligonucleotides targeting the transcription initiation site, e.g., between positions −10 and +10 from the start site are preferred. Similarly, inhibition of transcription can be achieved using oligonucleotides that base-pair with region(s) of the isogene DNA to form triplex DNA (see e.g., Gee et al. in Huber, B. E. and B. I. Carr, Molecular and Immunologic Approaches, Futura Publishing Co., Mt. Kisco, N.Y., 1994). Antisense oligonucleotides may also be designed to block translation of FCER1A mRNA transcribed from a particular isogene. It is also contemplated that ribozymes may be designed that can catalyze the specific cleavage of FCER1A mRNA transcribed from a particular isogene.

The untranslated mRNA, antisense RNA or antisense oligonucleotides may be delivered to a target cell or tissue by expression from a vector introduced into the cell or tissue in vivo or ex vivo. Alternatively, such molecules may be formulated as a pharmaceutical composition for administration to the patient. Oligoribonucleotides and/or oligodeoxynucleotides intended for use as antisense oligonucleotides may be modified to increase stability and half-life. Possible modifications include, but are not limited to phosphorothioate or 2′ O-methyl linkages, and the inclusion of nontraditional bases such as inosine and queosine, as well as acetyl-, methyl-, thio-, and similarly modified forms of adenine, cytosine, guanine, thymine, and uracil which are not as easily recognized by endogenous nucleases.

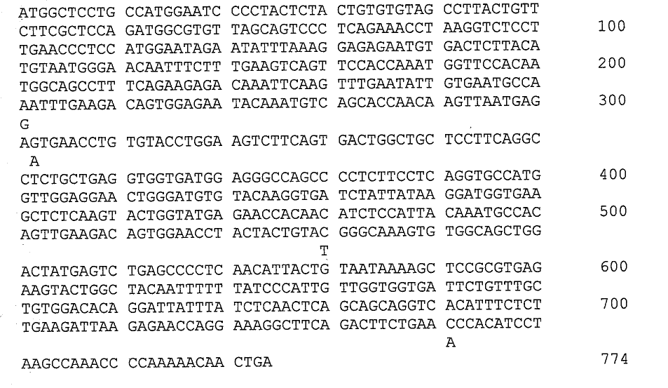

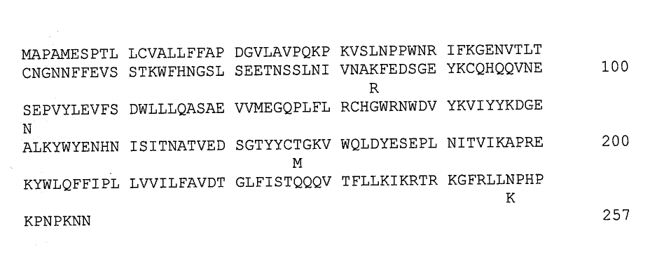

The invention also provides an isolated polypeptide comprising a polymorphic variant of (a) the reference FCER1A amino acid sequence shown in FIG. 3 or (b) a fragment of this reference sequence. The location of a variant amino acid in a FCER1A polypeptide or fragment of the invention is preferably identified by aligning its sequence against SEQ ID NO:3 (FIG. 3). A FCER1A protein variant of the invention comprises an amino acid sequence identical to SEQ ID NO:3 for those regions of SEQ ID NO:3 that are encoded by examined portions of the FCER1A gene (as described in the Examples below), except for having one or more variant amino acids selected from the group consisting of arginine at a position corresponding to amino acid position 84, asparagine at a position corresponding to amino acid position 101, methionine at a position corresponding to amino acid position 177 and lysine at a position corresponding to amino acid position 247. Thus, a FCER1A fragment of the invention, also referred to herein as a FCER1A peptide variant, is any fragment of a FCER1A protein variant that contains one or more of the amino acid variations shown in Table 2. The invention specifically excludes amino acid sequences identical to those previously identified for FCER1A, including SEQ ID NO:3, and previously described fragments thereof. FCER1A protein variants included within the invention comprise all amino acid sequences based on SEQ ID NO:3 and having the combination of amino acid variations described in Table 2 below. In preferred embodiments, a FCER1A protein variant of the invention is encoded by an isogene defined by one of the observed haplotypes, 7, 10, 12, 16, 17, and 19, shown in Table 5.

TABLE 2
Novel Polymorphic Variants of FCER1A

| | |
|---|---|
| Polymorphic | |
| Variant | Amino Acid Position and Identities |

| Number | 84 | 101 | 177 | 247 |
|---|---|---|---|---|
| 1 | K | S | T | K |
| 2 | K | S | M | N |
| 3 | K | S | M | K |
| 4 | K | N | T | N |
| 5 | K | N | T | K |
| 6 | K | N | M | N |
| 7 | K | N | M | K |
| 8 | R | S | T | N |
| 9 | R | S | T | K |
| 10 | R | S | M | N |
| 11 | R | S | M | K |
| 12 | R | N | T | N |
| 13 | R | N | T | K |
| 14 | R | N | M | N |
| 15 | R | N | M | K |

A FCER1A peptide variant of the invention is at least 6 amino acids in length and is preferably any number between 6 and 30 amino acids long, more preferably between 10 and 25, and most preferably between 15 and 20 amino acids long. Such FCER1A peptide variants may be useful as antigens to generate antibodies specific for one of the above FCER1A isoforms. In addition, the FCER1A peptide variants may be useful in drug screening assays.

A FCER1A variant protein or peptide of the invention may be prepared by chemical synthesis or by expressing an appropriate variant FCER1A genomic or cDNA sequence described above. Alternatively, the FCER1A protein variant may be isolated from a biological sample of an individual having a FCER1A isogene which encodes the variant protein. Where the sample contains two different FCER1A isoforms (i.e., the individual has different FCER1A isogenes), a particular FCER1A isoform of the invention can be isolated by immunoaffinity chromatography using an antibody which specifically binds to that particular FCER1A isoform but does not bind to the other FCER1A isoform.

The expressed or isolated FCER1A protein or peptide may be detected by methods known in the art, including Coomassie blue staining, silver staining, and Western blot analysis using antibodies specific for the isoform of the FCER1A protein or peptide as discussed further below. FCER1A variant proteins and peptides can be purified by standard protein purification procedures known in the art, including differential precipitation, molecular sieve chromatography, ion-exchange chromatography, isoelectric focusing, gel electrophoresis, affinity and immunoaffinity chromatography and the like. (Ausubel et. al., 1987, In Current Protocols in Molecular Biology John Wiley and Sons, New York, N.Y.). In the case of immunoaffinity chromatography, antibodies specific for a particular polymorphic variant may be used.

A polymorphic variant FCER1A gene of the invention may also be fused in frame with a heterologous sequence to encode a chimeric FCER1A protein. The non-FCER1A portion of the chimeric protein may be recognized by a commercially available antibody. In addition, the chimeric protein may also be engineered to contain a cleavage site located between the FCER1A and non-FCER1A portions so that the FCER1A protein may be cleaved and purified away from the non-FCER1A portion.

An additional embodiment of the invention relates to using a novel FCER1A protein isoform, or a fragment thereof, in any of a variety of drug screening assays. Such screening assays may be performed to identify agents that bind specifically to all known FCER1A protein isoforms or to only a subset of one or more of these isoforms. The agents may be from chemical compound libraries, peptide libraries and the like. The FCER1A protein or peptide variant may be free in solution or affixed to a solid support. In one embodiment, high throughput screening of compounds for binding to a FCER1A variant may be accomplished using the method described in PCT application WO84/03565, in which large numbers of test compounds are synthesized on a solid substrate, such as plastic pins or some other surface, contacted with the FCER1A protein(s) of interest and then washed. Bound FCER1A protein(s) are then detected using methods well-known in the art.

In another embodiment, a novel FCER1A protein isoform may be used in assays to measure the binding affinities of one or more candidate drugs targeting the FCER1A protein.

In yet another embodiment, when a particular FCER1A haplotype or group of FCER1A haplotypes encodes a FCER1A protein variant with an amino acid sequence distinct from that of FCER1A protein isoforms encoded by other FCER1A haplotypes, then detection of that particular FCER1A haplotype or group of FCER1A haplotypes may be accomplished by detecting expression of the encoded FCER1A protein variant using any of the methods described herein or otherwise commonly known to the skilled artisan.

In another embodiment, the invention provides antibodies specific for and immunoreactive with one or more of the novel FCER1A protein or peptide variants described herein. The antibodies may be either monoclonal or polyclonal in origin. The FCER1A protein or peptide variant used to generate the antibodies may be from natural or recombinant sources (in vitro or in vivo) or produced by chemical synthesis or semi-synthetic synthesis using synthesis techniques known in the art. If the FCER1A protein or peptide variant is of insufficient size to be antigenic, it may be concatenated or conjugated, complexed, or otherwise covalently linked to a carrier molecule to enhance the antigenicity of the peptide. Examples of carrier molecules, include, but are not limited to, albumins (e.g., human, bovine, fish, ovine), and keyhole limpet hemocyanin (Basic and Clinical Immunology, 1991, Eds. D. P. Stites, and A. I. Terr, Appleton and Lange, Norwalk Conn., San Mateo, Calif.).

In one embodiment, an antibody specifically immunoreactive with one of the novel protein or peptide variants described herein is administered to an individual to neutralize activity of the FCER1A isoform expressed by that individual. The antibody may be formulated as a pharmaceutical composition which includes a pharmaceutically acceptable carrier.

Antibodies specific for and immunoreactive with one of the novel protein isoforms described herein may be used to immunoprecipitate the FCER1A protein variant from solution as well as react with FCER1A protein isoforms on Western or immunoblots of polyacrylamide gels on membrane supports or substrates. In another preferred embodiment, the antibodies will detect FCER1A protein isoforms in paraffin or frozen tissue sections, or in cells which have been fixed or unfixed and prepared on slides, coverslips, or the like, for use in immunocytochemical, immunohistochemical, and immunofluorescence techniques.

In another embodiment, an antibody specifically immunoreactive with one of the novel FCER1A protein variants described herein is used in immunoassays to detect this variant in biological samples. In this method, an antibody of the present invention is contacted with a biological sample and the formation of a complex between the FCER1A protein variant and the antibody is detected. As described, suitable immunoassays include radioimmunoassay, Western blot assay, immunofluorescent assay, enzyme linked immunoassay (ELISA), chemiluminescent assay, immunohistochemical assay, immunocytochemical assay, and the like (see, e.g., Principles and Practice of Immunoassay, 1991, Eds. Christopher P. Price and David J. Neoman, Stockton Press, New York, N.Y.; Current Protocols in Molecular Biology, 1987, Eds. Ausubel et al., John Wiley and Sons, New York, N.Y.). Standard techniques known in the art for ELISA are described in Methods in Immunodiagnosis, 2nd Ed., Eds. Rose and Bigazzi, John Wiley and Sons, New York 1980; and Campbell et al., 1984, Methods in Immunology, W. A. Benjamin, Inc.). Such assays may be direct, indirect, competitive, or noncompetitive as described in the art (see, e.g., Principles and Practice of Immunoassay, 1991, Eds. Christopher P. Price and David J. Neoman, Stockton Pres, NY, N.Y.; and Oellirich, M., 1984, J. Clin. Chem. Clin. Biochem., 22:895-904). Proteins may be isolated from test specimens and biological samples by conventional methods, as described in Current Protocols in Molecular Biology, supra.

Exemplary antibody molecules for use in the detection and therapy methods of the present invention are intact immunoglobulin molecules, substantially intact immunoglobulin molecules, or those portions of immunoglobulin molecules that contain the antigen binding site. Polyclonal or monoclonal antibodies may be produced by methods conventionally known in the art (e.g., Kohler and Milstein, 1975, Nature, 256:495-497; Campbell Monoclonal Antibody Technology, the Production and Characterization of Rodent and Human Hybridomas, 1985, In: Laboratory Techniques in Biochemistry and Molecular Biology, Eds. Burdon et al., Volume 13, Elsevier Science Publishers, Amsterdam). The antibodies or antigen binding fragments thereof may also be produced by genetic engineering. The technology for expression of both heavy and light chain genes in E. coli is the subject of PCT patent applications, publication number WO 9014423 and WO 9014424 and in Huse et al., 1989, Science, 246:1275-1281. The antibodies may also be humanized (e.g., Queen, C. et al. 1989 Proc. Natl. Acad. Sci. USA 86;10029).

Effect(s) of the polymorphisms identified herein on expression of FCER1A may be investigated by various means known in the art, such as by in vitro translation of mRNA transcripts of the FCER1A gene, cDNA or fragment thereof, or by preparing recombinant cells and/or nonhuman recombinant organisms, preferably recombinant animals, containing a polymorphic variant of the FCER1A gene. As used herein, “expression” includes but is not limited to one or more of the following: transcription of the gene into precursor mRNA; splicing and other processing of the precursor mRNA to produce mature mRNA; mRNA stability; translation of the mature mRNA(s) into FCER1A protein(s) (including effects of polymorphisms on codon usage and tRNA availability); and glycosylation and/or other modifications of the translation product, if required for proper expression and function.

To prepare a recombinant cell of the invention, the desired FCER1A isogene, cDNA or coding sequence may be introduced into the cell in a vector such that the isogene, cDNA or coding sequence remains extrachromosomal. In such a situation, the gene will be expressed by the cell from the extrachromosomal location. In a preferred embodiment, the FCER1A isogene, cDNA or coding sequence is introduced into a cell in such a way that it recombines with the endogenous FCER1A gene present in the cell. Such recombination requires the occurrence of a double recombination event, thereby resulting in the desired FCER1A gene polymorphism. Vectors for the introduction of genes both for recombination and for extrachromosomal maintenance are known in the art, and any suitable vector or vector construct may be used in the invention. Methods such as electroporation, particle bombardment, calcium phosphate co-precipitation and viral transduction for introducing DNA into cells are known in the art; therefore, the choice of method may lie with the competence and preference of the skilled practitioner. Examples of cells into which the FCER1A isogene, cDNA or coding sequence may be introduced include, but are not limited to, continuous culture cells, such as COS, CHO, NIH/3T3, and primary or culture cells of the relevant tissue type, i.e., they express the FCER1A isogene, cDNA or coding sequence. Such recombinant cells can be used to compare the biological activities of the different protein variants.

Recombinant nonhuman organisms, i.e., transgenic animals, expressing a variant FCER1A gene, cDNA or coding sequence are prepared using standard procedures known in the art. Preferably, a construct comprising the variant gene, cDNA or coding sequence is introduced into a nonhuman animal or an ancestor of the animal at an embryonic stage, i.e., the one-cell stage, or generally not later than about the eight-cell stage. Transgenic animals carrying the constructs of the invention can be made by several methods known to those having skill in the art. One method involves transfecting into the embryo a retrovirus constructed to contain one or more insulator elements, a gene or genes (or cDNA or coding sequence) of interest, and other components known to those skilled in the art to provide a complete shuttle vector harboring the insulated gene(s) as a transgene, see e.g., U.S. Pat. No. 5,610,053. Another method involves directly injecting a transgene into the embryo. A third method involves the use of embryonic stem cells. Examples of animals into which the FCER1A isogene, cDNA or coding sequences may be introduced include, but are not limited to, mice, rats, other rodents, and nonhuman primates (see “The Introduction of Foreign Genes into Mice” and the cited references therein, In: Recombinant DNA, Eds. J. D. Watson, M. Gilman, J. Witkowski, and M. Zoller; W.H. Freeman and Company, New York, pages 254-272). Transgenic animals stably expressing a human FCER1A isogene, cDNA or coding sequence and producing the encoded human FCER1A protein can be used as biological models for studying diseases related to abnormal FCER1A expression and/or activity, and for screening and assaying various candidate drugs, compounds, and treatment regimens to reduce the symptoms or effects of these diseases.

An additional embodiment of the invention relates to pharmaceutical compositions for treating disorders affected by expression or function of a novel FCER1A isogene described herein. The pharmaceutical composition may comprise any of the following active ingredients: a polynucleotide comprising one of these novel FCER1A isogenes (or cDNAs or coding sequences); an antisense oligonucleotide directed against one of the novel FCER1A isogenes, a polynucleotide encoding such an antisense oligonucleotide, or another compound which inhibits expression of a novel FCER1A isogene described herein. Preferably, the composition contains the active ingredient in a therapeutically effective amount. By therapeutically effective amount is meant that one or more of the symptoms relating to disorders affected by expression or function of a novel FCER1A isogene is reduced and/or eliminated. The composition also comprises a pharmaceutically acceptable carrier, examples of which include, but are not limited to, saline, buffered saline, dextrose, and water. Those skilled in the art may employ a formulation most suitable for the active ingredient, whether it is a polynucleotide, oligonucleotide, protein, peptide or small molecule antagonist. The pharmaceutical composition may be administered alone or in combination with at least one other agent, such as a stabilizing compound. Administration of the pharmaceutical composition may be by any number of routes including, but not limited to oral, intravenous, intramuscular, intra-arterial, intramedullary, intrathecal, intraventricular, intradermal, transdermal, subcutaneous, intraperitoneal, intranasal, enteral, topical, sublingual, or rectal. Further details on techniques for formulation and administration may be found in the latest edition of Remington's Pharmaceutical Sciences (Maack Publishing Co., Easton, Pa.).

For any composition, determination of the therapeutically effective dose of active ingredient and/or the appropriate route of administration is well within the capability of those skilled in the art. For example, the dose can be estimated initially either in cell culture assays or in animal models. The animal model may also be used to determine the appropriate concentration range and route of administration. Such information can then be used to determine useful doses and routes for administration in humans. The exact dosage will be determined by the practitioner, in light of factors relating to the patient requiring treatment, including but not limited to severity of the disease state, general health, age, weight and gender of the patient, diet, time and frequency of administration, other drugs being taken by the patient, and tolerance/response to the treatment.

Any or all analytical and mathematical operations involved in practicing the methods of the present invention may be implemented by a computer. In addition, the computer may execute a program that generates views (or screens) displayed on a display device and with which the user can interact to view and analyze large amounts of information relating to the FCER1A gene and its genomic variation, including chromosome location, gene structure, and gene family, gene expression data, polymorphism data, genetic sequence data, and clinical data population data (e.g., data on ethnogeographic origin, clinical responses, genotypes, and haplotypes for one or more populations). The FCER1A polymorphism data described herein may be stored as part of a relational database (e.g., an instance of an Oracle database or a set of ASCII flat files). These polymorphism data may be stored on the computer's hard drive or may, for example, be stored on a CD-ROM or on one or more other storage devices accessible by the computer. For example, the data may be stored on one or more databases in communication with the computer via a network.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### EXAMPLES

The Examples herein are meant to exemplify the various aspects of carrying out the invention and are not intended to limit the scope of the invention in any way. The Examples do not include detailed descriptions for conventional methods employed, such as in the performance of genomic DNA isolation, PCR and sequencing procedures. Such methods are well-known to those skilled in the art and are described in numerous publications, for example, Sambrook, Fritsch, and Maniatis, “Molecular Cloning: A Laboratory Manual”, 2^{nd} Edition, Cold Spring Harbor Laboratory Press, USA, (1989).

### Example 1

This example illustrates examination of various regions of the FCER1A gene for polymorphic sites.

Amplification of Target Regions

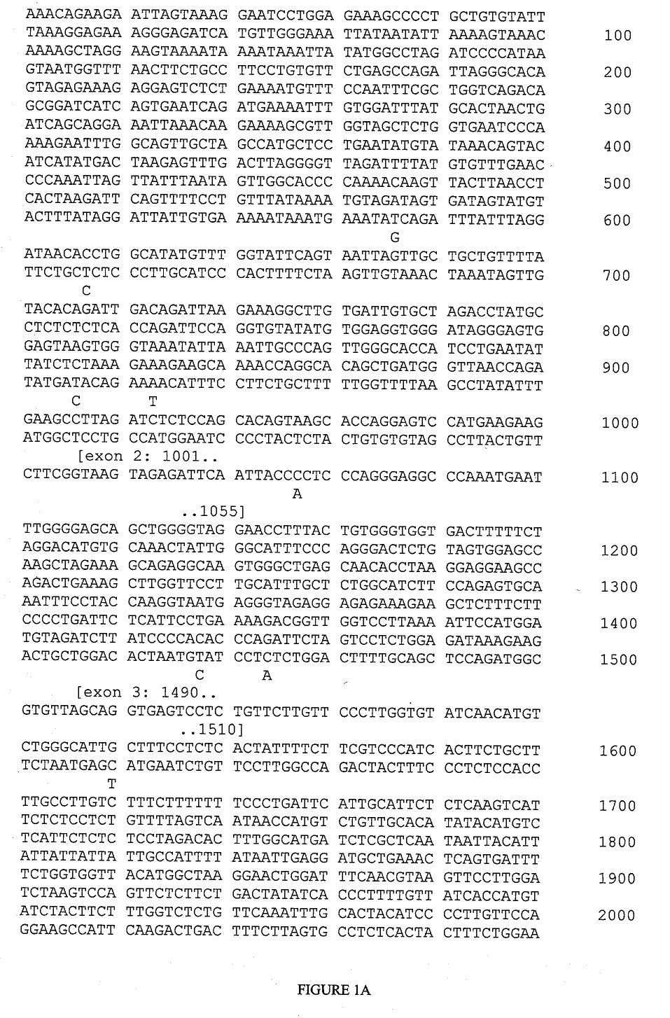

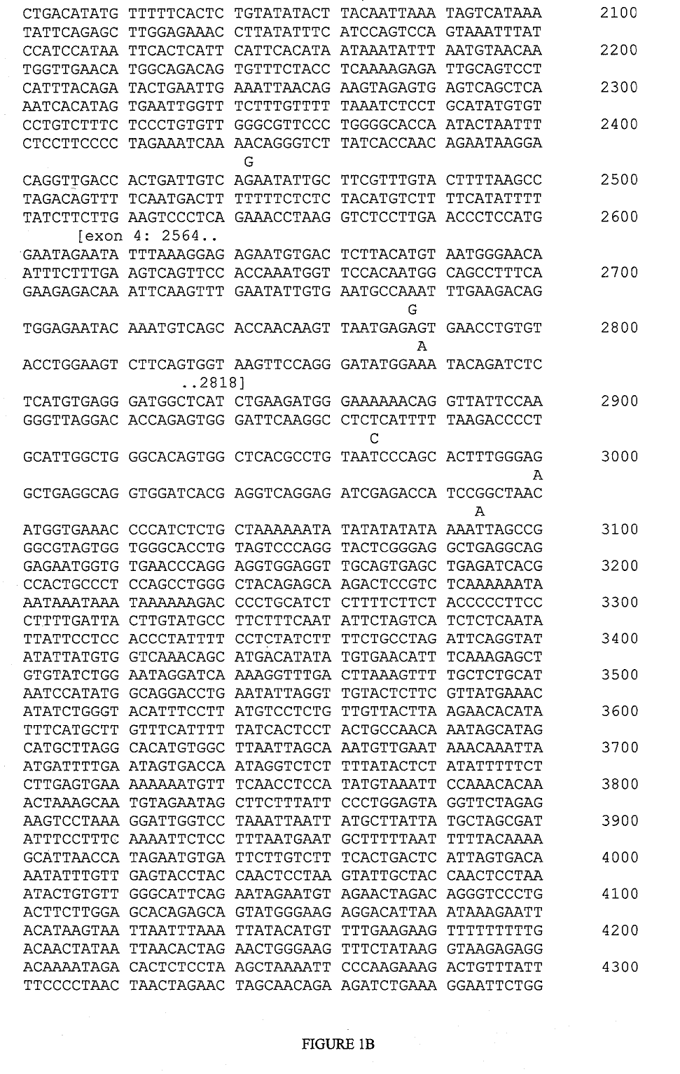

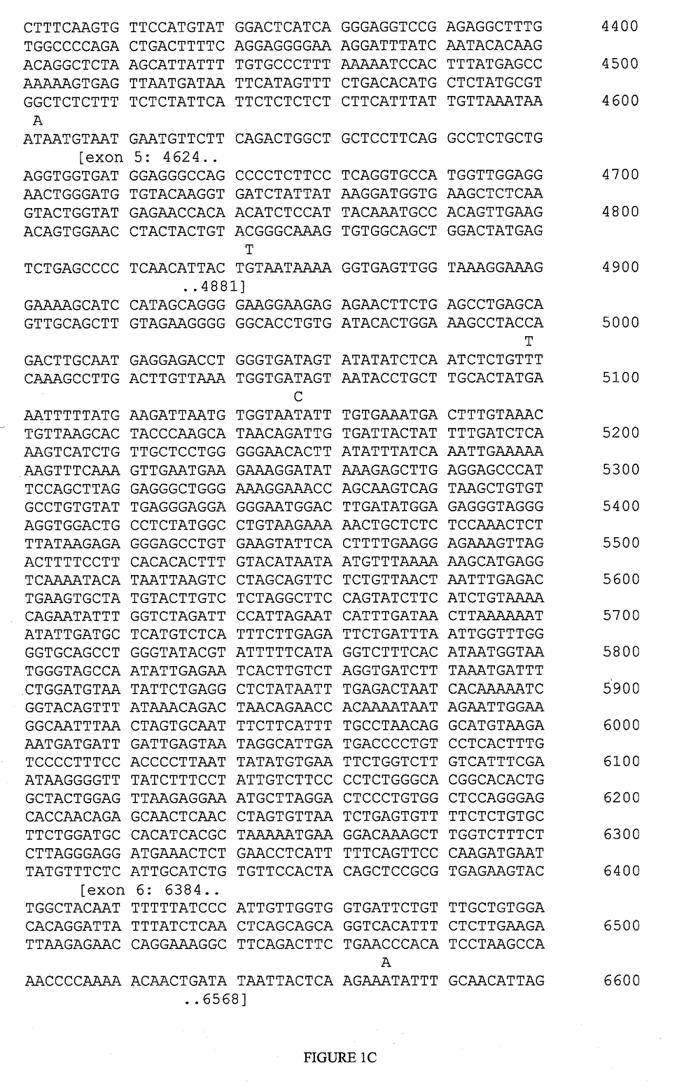

The following target regions of the FCER1A gene were amplified using PCR primer pairs. The primers used for each region are represented below by providing the nucleotide positions of their initial and final nucleotides, which correspond to positions in SEQ ID NO:1 (FIG. 1).

PCR Primer Pairs

| | | | PCR |
|---|---|---|---|
| Fragment No. | Forward Primer | Reverse Primer | Product |
| Fragment 1 | 319-341 | complement of 1138-1113 | 820 nt |
| Fragment 2 | 748-769 | complement of 1221-1199 | 474 nt |
| Fragment 3 | 788-810 | complement of 1331-1306 | 544 nt |
| Fragment 4 | 1319-1342 | complement of 1709-1684 | 391 nt |
| Fragment 5 | 2351-2372 | complement of 2919-2897 | 569 nt |
| Fragment 6 | 2553-2576 | complement of 3067-3045 | 515 nt |
| Fragment 7 | 4359-4382 | complement of 4932-4910 | 574 nt |
| Fragment 8 | 4527-4548 | complement of 5177-5157 | 651 nt |
| Fragment 9 | 6200-6221 | complement of 6926-6901 | 727 nt |
| Fragment 10 | 6423-6444 | complement of 7073-7050 | 651 nt |

These primer pairs were used in PCR reactions containing genomic DNA isolated from immortalized cell lines for each member of the Index Repository. The PCR reactions were carried out under the following conditions:

| | | |
|---|---|---|
| Reaction volume = | 10 | μl |
| 10 × Advantage 2 Polymerase reaction buffer (Clontech) = | 1 | μl |
| 100 ng of human genomic DNA = | 1 | μl |
| 10 mM dNTP = | 0.4 | μl |
| Advantage 2 Polymerase enzyme mix (Clontech) = | 0.2 | μl |
| Forward Primer (10 μM) = | 0.4 | μl |
| Reverse Primer (10 μM) = | 0.4 | μl |
| Water = | 6.6 | μl |

Amplification profile:
97° C.-2 min. 1 cycle

$$\begin{matrix}97 °C - 15 sec . \\ 70 °C - 45 sec . \\ 72 °C - 45 sec .\end{matrix} \} 10 cycles \\ \begin{matrix}97 °C - 15 sec . \\ 64 °C - 45 sec . \\ 72 °C - 45 sec .\end{matrix} \} 35 cycles$$

Sequencing of PCR Products

The PCR products were purified using a Whatman/Polyfiltronics 100 μl 384 well unifilter plate essentially according to the manufacturers protocol. The purified DNA was eluted in 50 μl of distilled water. Sequencing reactions were set up using Applied Biosystems Big Dye Terminator chemistry essentially according to the manufacturers protocol. The purified PCR products were sequenced in both directions using the primer sets described previously or those represented below by the nucleotide positions of their initial and final nucleotides, which correspond to positions in SEQ ID NO:1 (FIG. 1). Reaction products were purified by isopropanol precipitation, and run on an Applied Biosystems 3700 DNA Analyzer.

Sequencing Primer Pairs

| Fragment No. | Forward Primer | Reverse Primer |
|---|---|---|
| Fragment 1 | 470-490 | complement of 1013-994 |
| Fragment 2 | 782-801 | complement of 1185-1166 |
| Fragment 3 | 828-847 | complement of 1295-1275 |
| Fragment 4 | 1366-1387 | complement of 1656-1637 |
| Fragment 5 | 2375-2394 | complement of 2874-2854 |
| Fragment 6 | 2585-2606 | complement of 2960-2941 |
| Fragment 7 | 4399-4418 | complement of 4866-4847 |
| Fragment 8 | 4644-4664 | complement of 5025-5006 |
| Fragment 9 | 6255-6273 | complement of 6727-6706 |
| Fragment 10 | 6505-6526 | complement of 6998-6979 |

Analysis of Sequences for Polymorphic Sites

Sequence information for a minimum of 80 humans was analyzed for the presence of polymorphisms using the Polyphred program (Nickerson et al., Nucleic Acids Res. 14:2745-2751, 1997). The presence of a polymorphism was confirmed on both strands. The polymorphisms and their locations in the FCER1A reference genomic sequence (SEQ ID NO:1) are listed in Table 3 below.

TABLE 3
Polymorphic Sites Identified in the FCER1A Gene

| Polymorphic | | Nucleotide | Reference | Variant | CDS Variant | AA |
|---|---|---|---|---|---|---|
| Site Number | PolyId(a) | Position | Allele | Allele | Position | Variant |
| PS1 | 19003315 | 586 | T | G | | |
| PS2 | 19003411 | 657 | T | C | | |
| PS3 | 3179431 | 906 | T | C | | |
| PS4 | 3179433 | 913 | A | T | | |
| PS5 | 3179442 | 1077 | C | A | | |
| PS6 | 3179448 | 1468 | T | C | | |
| PS7 | 19004079 | 1474 | C | A | | |
| PS8 | 3179452 | 1610 | C | T | | |
| PS9 | 19004175 | 2422 | A | G | | |
| PS10 | 19004269 | 2738 | A | G | 251 | K84R |
| PS11 | 3179463 | 2789 | G | A | 302 | S101N |
| PS12 | 3179465 | 2934 | T | C | | |
| PS13 | 3179469 | 3000 | G | A | | |
| PS14 | 19004448 | 3044 | G | A | | |
| PS15 | 3179479 | 4552 | G | A | | |
| PS16 | 3179483 | 4822 | C | T | 530 | T177M |
| PS17 | 3179490 | 4999 | C | T | | |
| PS18 | 3179495 | 5077 | T | C | | |
| PS19 | 3179508 | 6535 | C | A | 741 | N247K |
| PS20 | 3179510 | 6625 | T | C | | |
| PS21 | 3179515 | 6650 | A | G | | |
| PS22 | 3179522 | 6714 | G | A | | |

(a)PolyId is a unique identifier assigned to each PS by Genaissance Pharmaceuticals, Inc.

### Example 2

This example illustrates analysis of the FCER1A polymorphisms identified in the Index Repository for human genotypes and haplotypes.

The different genotypes containing these polymorphisms that were observed in unrelated members of the reference population are shown in Table 4 below, with the haplotype pair indicating the combination of haplotypes determined for the individual using the haplotype derivation protocol described below. In Table 4, homozygous positions are indicated by one nucleotide and heterozygous positions are indicated by two nucleotides. Missing nucleotides in any given genotype in Table 4 were inferred based on linkage disequilibrium and/or Mendelian inheritance.

TABLE 4

(Part 1).
Genotypes and Haplotype Pairs
Observed in the IGERA Gene

Polymorphic Sites

| | | | | | | | | | | | | |
|---|---|---|---|---|---|---|---|---|---|---|---|---|
| Genotype | HAP | PS | PS | PS | PS | PS | PS | PS | PS | PS | PS | PS |
| Number | Pair | 1 | 2 | 3 | 4 | 5 | 6 | 7 | 8 | 9 | 10 | 11 |

| | | | | | | | | | | | | | |
|---|---|---|---|---|---|---|---|---|---|---|---|---|---|
| 1 | 1 | 1 | T | C | T | A | C | T | C | C | A | A | G |
| 2 | 1 | 2 | T | C/T | T | A | C | T | C | C | A | A | G |
| 3 | 1 | 3 | T | C/T | T | A | C | T | C | C | A | A | G |
| 4 | 1 | 4 | T | C | T/C | A | C | T | C | C | A | A | G |
| 5 | 1 | 5 | T/G | C | T | A | C | T | C | C | A | A | G |
| 6 | 1 | 7 | T | C | T | A | C | T | C | C/T | A | A/G | G |
| 7 | 1 | 11 | T/G | C | T | A | C | T/C | C | C | A | A | G |
| 8 | 1 | 12 | T | C | T | A | C | T | C | C | A | A | G |
| 9 | 1 | 15 | T | C | T | A | C | T | C/A | C/T | A | A | G |
| 10 | 1 | 16 | T | C | T | A | C | T | C | C | A | A | G |
| 11 | 1 | 17 | T | C | T | A | C | T | C | C/T | A | A/G | G |
| 12 | 1 | 20 | T | C/T | T | A | C/A | T | C | C/T | A | A | G |
| 13 | 2 | 2 | T | T | T | A | C | T | C | C | A | A | G |
| 14 | 2 | 3 | T | T | T | A | C | T | C | C | A | A | G |
| 15 | 2 | 4 | T | C/T | T/C | A | C | T | C | C | A | A | G |
| 16 | 2 | 6 | T | C/T | T | A | C | T | C | C/T | A | A | G |
| 17 | 2 | 9 | T | T | T | A | C | T | C | C | A/G | A | G |
| 18 | 2 | 10 | T | C/T | T | A | C | T | C | C/T | A | A | G/A |
| 19 | 2 | 13 | T | C/T | T | A | C | T | C | C/T | — | A | G |
| 20 | 2 | 14 | T | T | T | A | C | T | C | C/T | A | A | G |
| 21 | 3 | 3 | T | T | T | A | C | T | C | C | A | A | G |
| 22 | 3 | 4 | T | C/T | T/C | A | C | T | C | C | A | A | G |
| 23 | 3 | 5 | T/G | C/T | T | A | C | T | C | C | A | A | G |
| 24 | 3 | 6 | T | C/T | T | A | C | T | C | C/T | A | A | G |
| 25 | 3 | 9 | T | T | T | A | C | T | C | C | A/G | A | G |
| 26 | 3 | 12 | T | C/T | T | A | C | T | C | C | A | A | G |
| 27 | 3 | 15 | T | C/T | T | A | C | T | C/A | C/T | A | A | G |
| 28 | 3 | 19 | T | T | T | A | C | T | C | C | A | A | G |
| 29 | 4 | 4 | T | C | C | A | C | T | C | C | A | A | G |
| 30 | 4 | 5 | T/G | C | T/C | A | C | T | C | C | A | A | G |
| 31 | 4 | 6 | T | C | T/C | A | C | T | C | C/T | A | A | G |
| 32 | 4 | 8 | T | C | T/C | A | C | T | C | C/T | A | A | G |
| 33 | 4 | 11 | T/G | C | T/C | A | C | T/C | C | C | A | A | G |
| 34 | 4 | 13 | T | C | T/C | A | C | T | C | C/T | A | A | G |
| 35 | 5 | 5 | G | C | T | A | C | T | C | C | A | A | G |
| 36 | 5 | 11 | G | C | T | A | C | T/C | C | C | A | A | G |
| 37 | 5 | 15 | T/G | C | T | A | C | T | C/A | C/T | A | A | G |
| 38 | 6 | 6 | T | C | T | A | C | T | C | — | A | A | G |
| 39 | 6 | 7 | T | C | T | A | C | T | C | T | A | A/G | G |
| 40 | 6 | 8 | T | C | T | A | C | T | C | T | A | A | G |
| 41 | 6 | 10 | T | C | T | A | C | T | C | T | A | A | G/A |
| 42 | 6 | 18 | T | C | T | A/T | C | T | C | T | A | A | G |
| 43 | 7 | 7 | T | C | T | A | C | T | C | T | A | G | G |
| 44 | 7 | 10 | T | C | T | A | C | T | C | T | A | A/G | G/A |

(Part 2).
Genotypes and Haplotype Pairs
Observed in the IGERA Gene

Polymorphic Sites

| | | | | | | | | | | | | |
|---|---|---|---|---|---|---|---|---|---|---|---|---|
| Genotype | HAP | PS | PS | PS | PS | PS | PS | PS | PS | PS | PS | PS |
| Number | Pair | 12 | 13 | 14 | 15 | 16 | 17 | 18 | 19 | 20 | 21 | 22 |

| | | | | | | | | | | | | | |
|---|---|---|---|---|---|---|---|---|---|---|---|---|---|
| 1 | 1 | 1 | T | G | G | G | C | T | T | C | T | A | G |
| 2 | 1 | 2 | T | G | G | G | C | T/C | T | C | T | A | G |
| 3 | 1 | 3 | T | G | G | G | C | T/C | T | C | T | A | G/A |
| 4 | 1 | 4 | T | G | G | G | C | T | T | C | T | A | G |
| 5 | 1 | 5 | T | G | G | G | C | T | T | C | T | A | G |
| 6 | 1 | 7 | T | G | G | G/A | C | T | T/C | C | T | A/G | G |
| 7 | 1 | 11 | T/C | G | G | G | C | T | T | C | T/C | A | G |
| 8 | 1 | 12 | T | G | G | — | — | T | T | C/A | T | A | G |
| 9 | 1 | 15 | T | G | G | G | C | T | T | C | T | A | G |
| 10 | 1 | 16 | T | G | G/A | G | C | T | T | C/A | T | A | G |
| 11 | 1 | 17 | T | G | G | G | C | T | T/C | C | T | A | G |
| 12 | 1 | 20 | T | G | G | G/A | C | T/C | T/C | C | T | A | G |
| 13 | 2 | 2 | T | G | G | G | C | C | T | C | T | A | G |
| 14 | 2 | 3 | T | G | G | G | C | C | T | C | T | A | G/A |
| 15 | 2 | 4 | T | G | G | G | C | T/C | T | C | T | A | G |
| 16 | 2 | 6 | T | G | G | G/A | C | T/C | T/C | C | T | A | G |
| 17 | 2 | 9 | T | G | G | G | C | C | T | C | T | A | G |
| 18 | 2 | 10 | T | G/A | G | G | C | T/C | T | C | T | A | G |
| 19 | 2 | 13 | T | G | G | G/A | C | T/C | T/C | C | T | A/G | G |
| 20 | 2 | 14 | T | G | G | G | C | T/C | T | C | T | A | G/A |
| 21 | 3 | 3 | T | G | G | G | C | C | T | C | T | A | A |
| 22 | 3 | 4 | T | G | G | G | C | T/C | T | C | T | A | G/A |
| 23 | 3 | 5 | — | — | — | G | C | T/C | T | C | T | A | G/A |
| 24 | 3 | 6 | T | G | G | G/A | C | T/C | T/C | C | T | A | G/A |
| 25 | 3 | 9 | — | — | — | G | C | C | T | C | T | A | G/A |
| 26 | 3 | 12 | T | G | G | G | C | T/C | T | C/A | T | A | G/A |
| 27 | 3 | 15 | T | G | G | G | C | T/C | T | C | T | A | G/A |
| 28 | 3 | 19 | T | G | G | G | C/T | C | T | C | T | A | A |
| 29 | 4 | 4 | T | G | G | G | C | T | T | C | T | A | G |
| 30 | 4 | 5 | T | G | G | G | C | T | T | C | T | A | G |
| 31 | 4 | 6 | T | G | G | G/A | C | T | T/C | C | T | A | G |
| 32 | 4 | 8 | T | G | G | G | C | T | T | C | T | A | G |
| 33 | 4 | 11 | T/C | G | G | G | C | T | T | C | T/C | A | G |
| 34 | 4 | 13 | T | G | G | G/A | C | — | — | C | T | A/G | G |
| 35 | 5 | 5 | T | G | G | G | C | T | T | C | T | A | G |
| 36 | 5 | 11 | T/C | G | G | G | C | T | T | C | T/C | A | G |
| 37 | 5 | 15 | T | G | G | G | C | T | T | C | T | A | G |
| 38 | 6 | 6 | T | G | G | A | C | T | C | C | T | A | G |
| 39 | 6 | 7 | T | G | G | A | C | T | C | C | T | A/G | G |
| 40 | 6 | 8 | T | G | G | G/A | C | T | T/C | C | T | A | G |
| 41 | 6 | 10 | T | G/A | G | G/A | C | T | T/C | C | T | A | G |
| 42 | 6 | 18 | T | G | G | A | C | T | C | C | T | A | G |
| 43 | 7 | 7 | T | G | G | A | C | T | C | C | T | G | G |
| 44 | 7 | 10 | T | G/A | G | G/A | C | T | T/C | C | T | A/G | G |

The haplotype pairs shown in Table 4 were estimated from the unphased genotypes using a computer-implemented extension of Clark's algorithm (Clark, A. G. 1990 Mol Bio Evol 7, 111-122) for assigning haplotypes to unrelated individuals in a population sample, as described in PCT/US01/12831, filed Apr. 18, 2001. In this method, haplotypes are assigned directly from individuals who are homozygous at all sites or heterozygous at no more than one of the variable sites. This list of haplotypes is then used to deconvolute the unphased genotypes in the remaining (multiply heterozygous) individuals. In the present analysis, the list of haplotypes was augmented with haplotypes obtained from two families (one three-generation Caucasian family and one two-generation African-American family).

By following this protocol, it was determined that the Index Repository examined herein and, by extension, the general population contains the 22 human FCER1A haplotypes shown in Table 5 below.

A FCER1A isogene defined by a full-haplotype shown in Table 5 below comprises the regions of the SEQ ID NOS indicated in Table 5, with their corresponding set of polymorphic locations and identities, which are also set forth in Table 5.

TABLE 5

(Part 1).
Haplotypes Observed in the FCER1A Gene

| Regions | PS | PS | Haplotype Number(d) | | | | | | | | | |
|---|---|---|---|---|---|---|---|---|---|---|---|---|
| Examined(a) | Number(b) | Position(c) | 1 | 2 | 3 | 4 | 5 | 6 | 7 | 8 | 9 | 10 |

| | | | | | | | | | | | | |
|---|---|---|---|---|---|---|---|---|---|---|---|---|
| 319-1709 | PS1 | 586/30 | T | T | T | T | G | T | T | T | T | T |
| 319-1709 | PS2 | 657/150 | C | T | T | C | C | C | C | C | T | C |
| 319-1709 | PS3 | 906/270 | T | T | T | C | T | T | T | T | T | T |
| 319-1709 | PS4 | 913/390 | A | A | A | A | A | A | A | A | A | A |
| 319-1709 | PS5 | 1077/510 | C | C | C | C | C | C | C | C | C | C |
| 319-1709 | PS6 | 1468/630 | T | T | T | T | T | T | T | T | T | T |
| 319-1709 | PS7 | 1474/750 | C | C | C | C | C | C | C | C | C | C |
| 319-1709 | PS8 | 1610/870 | C | C | C | C | C | T | T | T | C | T |
| 2351-3067 | PS9 | 2422/990 | A | A | A | A | A | A | A | A | G | A |
| 2351-3067 | PS10 | 2738/1110 | A | A | A | A | A | A | G | A | A | A |
| 2351-3067 | PS11 | 2789/1230 | G | G | G | G | G | G | G | G | G | A |
| 2351-3067 | PS12 | 2934/1350 | T | T | T | T | T | T | T | T | T | T |
| 2351-3067 | PS13 | 3000/1470 | G | G | G | G | G | G | G | G | G | A |
| 2351-3067 | PS14 | 3044/1590 | G | G | G | G | G | G | G | G | G | G |
| 4359-5177 | PS15 | 4552/1710 | G | G | G | G | G | A | A | G | G | G |
| 4359-5177 | PS16 | 4822/1830 | C | C | C | C | C | C | C | C | C | C |
| 4359-5177 | PS17 | 4999/1950 | T | C | C | T | T | T | T | T | C | T |
| 4359-5177 | PS18 | 5077/2070 | T | T | T | T | T | C | C | T | T | T |
| 6200-7073 | PS19 | 6535/2190 | C | C | C | C | C | C | C | C | C | C |
| 6200-7073 | PS20 | 6625/2310 | T | T | T | T | T | T | T | T | T | T |
| 6200-7073 | PS21 | 6650/2430 | A | A | A | A | A | A | G | A | A | A |
| 6200-7073 | PS22 | 6714/2550 | G | G | A | G | G | G | G | G | G | G |

(Part 2).
Haplotypes Observed in the FCER1A Gene

| Regions | PS | PS | Haplotype Number(d) | | | | | | | | | |
|---|---|---|---|---|---|---|---|---|---|---|---|---|
| Examined(a) | Number(b) | Position(c) | 11 | 12 | 13 | 14 | 15 | 16 | 17 | 18 | 19 | 20 |

| | | | | | | | | | | | | |
|---|---|---|---|---|---|---|---|---|---|---|---|---|
| 319-1709 | PS1 | 586/30 | G | T | T | T | T | T | T | T | T | T |
| 319-1709 | PS2 | 657/150 | C | C | C | T | C | C | C | C | T | T |
| 319-1709 | PS3 | 906/270 | T | T | T | T | T | T | T | T | T | T |
| 319-1709 | PS4 | 913/390 | A | A | A | A | A | A | A | T | A | A |
| 319-1709 | PS5 | 1077/510 | C | C | C | C | C | C | C | C | C | A |
| 319-1709 | PS6 | 1468/630 | C | T | T | T | T | T | T | T | T | T |
| 319-1709 | PS7 | 1474/750 | C | C | C | C | A | C | C | C | C | C |
| 319-1709 | PS8 | 1610/870 | C | C | T | T | T | C | T | T | C | T |
| 2351-3067 | PS9 | 2422/990 | A | A | A | A | A | A | A | A | A | A |
| 2351-3067 | PS10 | 2738/1110 | A | A | A | A | A | A | G | A | A | A |
| 2351-3067 | PS11 | 2789/1230 | G | G | G | G | G | G | G | G | G | G |
| 2351-3067 | PS12 | 2934/1350 | C | T | T | T | T | T | T | T | T | T |
| 2351-3067 | PS13 | 3000/1470 | G | G | G | G | G | G | G | G | G | G |
| 2351-3067 | PS14 | 3044/1590 | G | G | G | G | G | A | G | G | G | G |
| 4359-5177 | PS15 | 4552/1710 | G | G | A | G | G | G | G | A | G | A |
| 4359-5177 | PS16 | 4822/1830 | C | C | C | C | C | C | C | C | T | C |
| 4359-5177 | PS17 | 4999/1950 | T | T | T | C | T | T | T | T | C | C |
| 4359-5177 | PS18 | 5077/2070 | T | T | C | T | T | T | T | C | T | C |
| 6200-7073 | PS19 | 6535/2190 | C | A | C | C | C | A | C | C | C | C |
| 6200-7073 | PS20 | 6625/2310 | C | T | T | T | T | T | T | T | T | T |
| 6200-7073 | PS21 | 6650/2430 | A | A | G | A | A | A | A | A | A | A |
| 6200-7073 | PS22 | 6714/2550 | G | G | G | A | G | G | G | G | A | G |

(a)Region examined represents the nucleotide positions defining the start and stop positions within SEQ ID NO: 1 of the regions sequenced;
(b)PS = polymorphic site;
(c)Position of PS within the indicated SEQ ID NO, with the 1^{st} position number referring to SEQ ID NO: 1 and the 2^{nd} position number referring to SEQ ID NO:114, a modified version of SEQ ID NO: 1 that comprises the context sequence of each polymorphic site, PS1-PS22, to facilitate electronic searching of the haplotypes;
(d)Alleles for FCER1A haplotypes are presented 5′ to 3′ in each column.

SEQ ID NO:1 refers to FIG. 1, with the two alternative allelic variants of each polymorphic site indicated by the appropriate nucleotide symbol. SEQ ID NO:114 is a modified version of SEQ ID NO:1 that shows the context sequence of each of PS1-PS22 in a uniform format to facilitate electronic searching of the FCER1A haplotypes. For each polymorphic site, SEQ ID NO:114 contains a block of 60 bases of the nucleotide sequence encompassing the centrally-located polymorphic site at the 30^{th} position, followed by 60 bases of unspecified sequence to represent that each polymorphic site is separated by genomic sequence whose composition is defined elsewhere herein.

Table 6 below shows the percent of chromosomes characterized by a given FCER1A haplotype for all unrelated individuals in the Index Repository for which haplotype data was obtained. The percent of these unrelated individuals who have a given FCER1A haplotype pair is shown in Table 7. In Tables 6 and 7, the “Total” column shows this frequency data for all of these unrelated individuals, while the other columns show the frequency data for these unrelated individuals categorized according to their self-identified ethnogeographic origin. Abbreviations used in Tables 6 and 7 are AF=African Descent, AS=Asian, CA=Caucasian, HL=Hispanic-Latino, and AM=Native American.

TABLE 6
Frequency of Observed FCER1A Haplotypes
In Unrelated Individuals

| HAP No. | Total | CA | AF | AS | HL | AM |
|---|---|---|---|---|---|---|
| 1 | 18.9 | 7.14 | 25 | 20 | 22.22 | 33.33 |
| 2 | 17.07 | 26.19 | 0 | 25 | 16.67 | 16.67 |
| 3 | 14.63 | 21.43 | 7.5 | 0 | 27.78 | 33.33 |
| 4 | 13.41 | 19.05 | 15 | 2.5 | 19.44 | 0 |
| 5 | 9.15 | 0 | 35 | 0 | 2.78 | 0 |
| 6 | 9.15 | 14.29 | 2.5 | 20 | 0 | 0 |
| 7 | 3.66 | 0 | 0 | 15 | 0 | 0 |
| 8 | 1.22 | 2.38 | 2.5 | 0 | 0 | 0 |
| 9 | 1.83 | 2.38 | 0 | 0 | 5.56 | 0 |
| 10 | 1.83 | 0 | 0 | 7.5 | 0 | 0 |
| 11 | 1.83 | 0 | 7.5 | 0 | 0 | 0 |
| 12 | 1.22 | 0 | 0 | 0 | 5.56 | 0 |
| 13 | 1.22 | 0 | 0 | 5 | 0 | 0 |
| 14 | 1.22 | 4.76 | 0 | 0 | 0 | 0 |
| 15 | 0.61 | 0 | 2.5 | 0 | 0 | 0 |
| 16 | 0.61 | 0 | 2.5 | 0 | 0 | 0 |
| 17 | 0.61 | 0 | 0 | 2.5 | 0 | 0 |
| 18 | 0.61 | 0 | 0 | 2.5 | 0 | 0 |
| 19 | 0.61 | 0 | 0 | 0 | 0 | 16.67 |
| 20 | 0.61 | 2.38 | 0 | 0 | 0 | 0 |

TABLE 7
Frequency of Observed FCER1A Haplotype
Pairs In Unrelated Individuals

| HAP1 | HAP2 | Total | CA | AF | AS | HL | AM |
|---|---|---|---|---|---|---|---|
| 1 | 1 | 3.66 | 0 | 0 | 10 | 0 | 33.33 |
| 1 | 2 | 6.1 | 4.76 | 0 | 5 | 16.67 | 0 |
| 1 | 3 | 3.66 | 4.76 | 0 | 0 | 11.11 | 0 |
| 1 | 4 | 3.66 | 0 | 5 | 0 | 11.11 | 0 |
| 1 | 5 | 8.54 | 0 | 35 | 0 | 0 | 0 |
| 1 | 7 | 2.44 | 0 | 0 | 10 | 0 | 0 |
| 1 | 11 | 1.22 | 0 | 5 | 0 | 0 | 0 |
| 1 | 12 | 1.22 | 0 | 0 | 0 | 5.56 | 0 |
| 1 | 16 | 1.22 | 0 | 5 | 0 | 0 | 0 |
| 1 | 17 | 1.22 | 0 | 0 | 5 | 0 | 0 |
| 1 | 20 | 1.22 | 4.76 | 0 | 0 | 0 | 0 |
| 2 | 2 | 3.66 | 0 | 0 | 15 | 0 | 0 |
| 2 | 3 | 6.1 | 9.52 | 0 | 0 | 11.11 | 33.33 |
| 2 | 4 | 4.88 | 19.05 | 0 | 0 | 0 | 0 |
| 2 | 6 | 2.44 | 4.76 | 0 | 5 | 0 | 0 |
| 2 | 9 | 2.44 | 4.76 | 0 | 0 | 5.56 | 0 |
| 2 | 10 | 1.22 | 0 | 0 | 5 | 0 | 0 |
| 2 | 13 | 1.22 | 0 | 0 | 5 | 0 | 0 |
| 2 | 14 | 2.44 | 9.52 | 0 | 0 | 0 | 0 |
| 3 | 3 | 2.44 | 0 | 5 | 0 | 5.56 | 0 |
| 3 | 4 | 4.88 | 14.29 | 0 | 0 | 5.56 | 0 |
| 3 | 5 | 1.22 | 0 | 0 | 0 | 5.56 | 0 |
| 3 | 6 | 3.66 | 14.29 | 0 | 0 | 0 | 0 |
| 3 | 9 | 1.22 | 0 | 0 | 0 | 5.56 | 0 |
| 3 | 12 | 1.22 | 0 | 0 | 0 | 5.56 | 0 |
| 3 | 15 | 1.22 | 0 | 5 | 0 | 0 | 0 |
| 3 | 19 | 1.22 | 0 | 0 | 0 | 0 | 33.33 |
| 4 | 4 | 2.44 | 0 | 0 | 0 | 11.11 | 0 |
| 4 | 5 | 4.88 | 0 | 20 | 0 | 0 | 0 |
| 4 | 8 | 1.22 | 4.76 | 0 | 0 | 0 | 0 |
| 4 | 11 | 1.22 | 0 | 5 | 0 | 0 | 0 |
| 4 | 13 | 1.22 | 0 | 0 | 5 | 0 | 0 |
| 5 | 5 | 1.22 | 0 | 5 | 0 | 0 | 0 |
| 5 | 11 | 1.22 | 0 | 5 | 0 | 0 | 0 |
| 6 | 6 | 3.66 | 4.76 | 0 | 10 | 0 | 0 |
| 6 | 7 | 1.22 | 0 | 0 | 5 | 0 | 0 |
| 6 | 8 | 1.22 | 0 | 5 | 0 | 0 | 0 |
| 6 | 10 | 1.22 | 0 | 0 | 5 | 0 | 0 |
| 6 | 18 | 1.22 | 0 | 0 | 5 | 0 | 0 |
| 7 | 7 | 1.22 | 0 | 0 | 5 | 0 | 0 |
| 7 | 10 | 1.22 | 0 | 0 | 5 | 0 | 0 |

The size and composition of the Index Repository were chosen to represent the genetic diversity across and within four major population groups comprising the general United States population. For example, as described in Table 1 above, this repository contains approximately equal sample sized of African-descent, Asian-American, European-American, and Hispanic-Latino population groups. Almost all individuals representing each group had all four grandparents with the same ethnogeographic background. The number of unrelated individuals in the Index Repository provides a sample size that is sufficient to detect SNPs and haplotypes that occur in the general population with high statistical certainty. For instance, a haplotype that occurs with a frequency of 5% in the general population has a probability higher than 99.9% of being observed in a sample of 80 individuals from the general population. Similarly, a haplotype that occurs with a frequency of 10% in a specific population group has a 99% probability of being observed in a sample of 20 individuals from that population group. In addition, the size and composition of the Index Repository means that the relative frequencies determined therein for the haplotypes and haplotype pairs of the FCER1A gene are likely to be similar to the relative frequencies of these FCER1A haplotypes and haplotype pairs in the general U.S. population and in the four population groups represented in the Index Repository. The genetic diversity observed for the three Native Americans is presented because it is of scientific interest, but due to the small sample size it lacks statistical significance.

In view of the above, it will be seen that the several advantages of the invention are achieved and other advantageous results attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including patents and patent applications, are hereby incorporated in their entirety by reference. The discussion of references herein is intended merely to summarize the assertions made by their authors and no admission is made that any reference constitutes prior art. Applicants reserve the right to challenge the accuracy and pertinency of the cited references.

1. A method for haplotyping the Fc fragment of IgE, high affinity I, receptor for; alpha polypeptide (FCER1A) gene of an individual, which comprises determining which of the FCER1A haplotypes shown in the table immediately below defines one copy of the individual's FCER1A gene, wherein the determining step comprises identifying the phased sequence of nucleotides present at each of PS1-PS22 on at least one copy of the individual's FCER1A gene, and wherein each of the FCER1A haplotypes comprises a sequence of polymorphisms whose positions and identities are set forth in the table immediately below:
PS

| Num- | PS | Haplotype Number(c) | | | | | | | | | |
|---|---|---|---|---|---|---|---|---|---|---|---|
| ber(a) | Position(b) | 1 | 2 | 3 | 4 | 5 | 6 | 7 | 8 | 9 | 10 |
| | | | | | | | | | | | |
|---|---|---|---|---|---|---|---|---|---|---|---|
| PS1 | 586 | T | T | T | T | G | T | T | T | T | T |
| PS2 | 657 | C | T | T | C | C | C | C | C | T | C |
| PS3 | 906 | T | T | T | C | T | T | T | T | T | T |
| PS4 | 913 | A | A | A | A | A | A | A | A | A | A |
| PS5 | 1077 | C | C | C | C | C | C | C | C | C | C |
| PS6 | 1468 | T | T | T | T | T | T | T | T | T | T |
| PS7 | 1474 | C | C | C | C | C | C | C | C | C | C |
| PS8 | 1610 | C | C | C | C | C | T | T | T | C | T |
| PS9 | 2422 | A | A | A | A | A | A | A | A | G | A |
| PS10 | 2738 | A | A | A | A | A | A | G | A | A | A |
| PS11 | 2789 | G | G | G | G | G | G | G | G | G | A |
| PS12 | 2934 | T | T | T | T | T | T | T | T | T | T |
| PS13 | 3000 | G | G | G | G | G | G | G | G | G | A |
| PS14 | 3044 | G | G | G | G | G | G | G | G | G | G |
| PS15 | 4552 | G | G | G | G | G | A | A | G | G | G |
| PS16 | 4822 | C | C | C | C | C | C | C | C | C | C |
| PS17 | 4999 | T | C | C | T | T | T | T | T | C | T |
| PS18 | 5077 | T | T | T | T | T | C | C | T | T | T |
| PS19 | 6535 | C | C | C | C | C | C | C | C | C | C |
| PS20 | 6625 | T | T | T | T | T | T | T | T | T | T |
| PS21 | 6650 | A | A | A | A | A | A | G | A | A | A |
| PS22 | 6714 | G | G | A | G | G | G | G | G | G | G |

PS

| Num- | PS | Haplotype Number(c) | | | | | | | | | |
|---|---|---|---|---|---|---|---|---|---|---|---|
| ber(a) | Position(b) | 11 | 12 | 13 | 14 | 15 | 16 | 17 | 18 | 19 | 20 |
| | | | | | | | | | | | |
|---|---|---|---|---|---|---|---|---|---|---|---|
| PS1 | 586 | G | T | T | T | T | T | T | T | T | T |
| PS2 | 657 | C | C | C | T | C | C | C | C | T | T |
| PS3 | 906 | T | T | T | T | T | T | T | T | T | T |
| PS4 | 913 | A | A | A | A | A | A | A | T | A | A |
| PS5 | 1077 | C | C | C | C | C | C | C | C | C | A |
| PS6 | 1468 | C | T | T | T | T | T | T | T | T | T |
| PS7 | 1474 | C | C | C | C | A | C | C | C | C | C |
| PS8 | 1610 | C | C | T | T | T | C | T | T | C | T |
| PS9 | 2422 | A | A | A | A | A | A | A | A | A | A |
| PS10 | 2738 | A | A | A | A | A | A | G | A | A | A |
| PS11 | 2789 | G | G | G | G | G | G | G | G | G | G |
| PS12 | 2934 | C | T | T | T | T | T | T | T | T | T |
| PS13 | 3000 | G | G | G | G | G | G | G | G | G | G |
| PS14 | 3044 | G | G | G | G | G | A | G | G | G | G |
| PS15 | 4552 | G | G | A | G | G | G | G | A | G | A |
| PS16 | 4822 | C | C | C | C | C | C | C | C | T | C |
| PS17 | 4999 | T | T | T | C | T | T | T | T | C | C |
| PS18 | 5077 | T | T | C | T | T | T | T | C | T | C |
| PS19 | 6535 | C | A | C | C | C | A | C | C | C | C |
| PS20 | 6625 | C | T | T | T | T | T | T | T | T | T |
| PS21 | 6650 | A | A | G | A | A | A | A | A | A | A |
| PS22 | 6714 | G | G | G | A | G | G | G | G | A | G |

(a)PS = polymorphic site;
(b)Position of PS within SEQ ID NO:1;
(c)Alleles for haplotypes are presented 5′ to 3′ in each column.

2. A method for haplotyping the Fc fragment of IgE, high affinity I, receptor for; alpha polypeptide (FCER1A) gene of an individual, which comprises determining which of the FCER1A haplotype pairs shown in the table immediately below defines both copies of the individual's FCER1A gene, wherein the determining step comprises identifying the phased sequence of nucleotides present at each of PS1-PS22 on both copies of the individual's FCER1A gene, and wherein each of the FCER1A haplotype pairs consists of first and second haplotypes which comprise first and second sequences of polymorphisms whose positions and identities are set forth in the table immediately below:

| PS | PS | Haplotype Pair(c) Part 1 | | | | | | | | | | | | | | |
|---|---|---|---|---|---|---|---|---|---|---|---|---|---|---|---|---|
| Number(a) | Position(b) | 1/1 | 1/2 | 1/3 | 1/4 | 1/5 | 1/7 | 1/11 | 1/12 | 1/15 | 1/16 | 1/17 | 1/20 | 2/2 | 2/3 | 2/4 |
| PS1 | 586 | T | T | T | T | T/G | T | T/G | T | T | T | T | T | T | T | T |
|---|---|---|---|---|---|---|---|---|---|---|---|---|---|---|---|---|
| PS2 | 657 | C | C/T | C/T | C | C | C | C | C | C | C | C | C/T | T | T | C/T |
| PS3 | 906 | T | T | T | T/C | T | T | T | T | T | T | T | T | T | T | T/C |
| PS4 | 913 | A | A | A | A | A | A | A | A | A | A | A | A | A | A | A |
| PS5 | 1077 | C | C | C | C | C | C | C | C | C | C | C | C/A | C | C | C |
| PS6 | 1468 | T | T | T | T | T | T | T/C | T | T | T | T | T | T | T | T |
| PS7 | 1474 | C | C | C | C | C | C | C | C | C/A | C | C | C | C | C | C |
| PS8 | 1610 | C | C | C | C | C | C/T | C | C | C/T | C | C/T | C/T | C | C | C |
| PS9 | 2422 | A | A | A | A | A | A | A | A | A | A | A | A | A | A | A |
| PS10 | 2738 | A | A | A | A | A | A/G | A | A | A | A | A/G | A | A | A | A |
| PS11 | 2789 | G | G | G | G | G | G | G | G | G | G | G | G | G | G | G |
| PS12 | 2934 | T | T | T | T | T | T | T/C | T | T | T | T | T | T | T | T |
| PS13 | 3000 | G | G | G | G | G | G | G | G | G | G | G | G | G | G | G |
| PS14 | 3044 | G | G | G | G | G | G | G | G | G | G/A | G | G | G | G | G |
| PS15 | 4552 | G | G | G | G | G | G/A | G | G | G | G | G | G/A | G | G | G |
| PS16 | 4822 | C | C | C | C | C | C | C | C | C | C | C | C | C | C | C |
| PS17 | 4999 | T | T/C | T/C | T | T | T | T | T | T | T | T | T/C | C | C | T/C |
| PS18 | 5077 | T | T | T | T | T | T/C | T | T | T | T | T/C | T/C | T | T | T |
| PS19 | 6535 | C | C | C | C | C | C | C | C/A | C | C/A | C | C | C | C | C |
| PS20 | 6625 | T | T | T | T | T | T | T/C | T | T | T | T | T | T | T | T |
| PS21 | 6650 | A | A | A | A | A | A/G | A | A | A | A | A | A | A | A | A |
| PS22 | 6714 | G | G | G/A | G | G | G | G | G | G | G | G | G | G | G/A | G |
| PS | PS | Haplotype Pair(c) Part 2 | | | | | | | | | | | | | | |
|---|---|---|---|---|---|---|---|---|---|---|---|---|---|---|---|---|
| Number(a) | Position(b) | 2/6 | 2/9 | 2/10 | 2/13 | 2/14 | 3/3 | 3/4 | 3/5 | 3/6 | 3/9 | 3/12 | 3/15 | 3/19 | 4/4 | 4/5 |
| | | | | | | | | | | | | | | | | |
|---|---|---|---|---|---|---|---|---|---|---|---|---|---|---|---|---|
| PS1 | 586 | T | T | T | T | T | T | T | T/G | T | T | T | T | T | T | T/G |
| PS2 | 657 | C/T | T | C/T | C/T | T | T | C/T | C/T | C/T | T | C/T | C/T | T | C | C |
| PS3 | 906 | T | T | T | T | T | T | T/C | T | T | T | T | T | T | C | T/C |
| PS4 | 913 | A | A | A | A | A | A | A | A | A | A | A | A | A | A | A |
| PS5 | 1077 | C | C | C | C | C | C | C | C | C | C | C | C | C | C | C |
| PS6 | 1468 | T | T | T | T | T | T | T | T | T | T | T | T | T | T | T |
| PS7 | 1474 | C | C | C | C | C | C | C | C | C | C | C | C/A | C | C | C |
| PS8 | 1610 | C/T | C | C/T | C/T | C/T | C | C | C | C/T | C | C | C/T | C | C | C |
| PS9 | 2422 | A | A/G | A | A | A | A | A | A | A | A/G | A | A | A | A | A |
| PS10 | 2738 | A | A | A | A | A | A | A | A | A | A | A | A | A | A | A |
| PS11 | 2789 | G | G | G/A | G | G | G | G | G | G | G | G | G | G | G | G |
| PS12 | 2934 | T | T | T | T | T | T | T | T | T | T | T | T | T | T | T |
| PS13 | 3000 | G | G | G/A | G | G | G | G | G | G | G | G | G | G | G | G |
| PS14 | 3044 | G | G | G | G | G | G | G | G | G | G | G | G | G | G | G |
| PS15 | 4552 | G/A | G | G | G/A | G | G | G | G | G/A | G | G | G | G | G | G |
| PS16 | 4822 | C | C | C | C | C | C | C | C | C | C | C | C | C/T | C | C |
| PS17 | 4999 | T/C | C | T/C | T/C | T/C | C | T/C | T/C | T/C | C | T/C | T/C | C | T | T |
| PS18 | 5077 | T/C | T | T | T/C | T | T | T | T | T/C | T | T | T | T | T | T |
| PS19 | 6535 | C | C | C | C | C | C | C | C | C | C | C/A | C | C | C | C |
| PS20 | 6625 | T | T | T | T | T | T | T | T | T | T | T | T | T | T | T |
| PS21 | 6650 | A | A | A | A/G | A | A | A | A | A | A | A | A | A | A | A |
| PS22 | 6714 | G | G | G | G | G/A | A | G/A | G/A | G/A | G/A | G/A | G/A | A | G | G |
| PS | PS | Haplotype Pair(c) Part 3 | | | | | | | | | | | | | |
|---|---|---|---|---|---|---|---|---|---|---|---|---|---|---|---|
| Number(a) | Position(b) | 4/6 | 4/8 | 4/11 | 4/13 | 5/5 | 5/11 | 5/15 | 6/6 | 6/7 | 6/8 | 6/10 | 6/18 | 7/7 | 7/10 |
| | | | | | | | | | | | | | | | |
|---|---|---|---|---|---|---|---|---|---|---|---|---|---|---|---|
| PS1 | 586 | T | T | T/G | T | G | G | T/G | T | T | T | T | T | T | T |
| PS2 | 657 | C | C | C | C | C | C | C | C | C | C | C | C | C | C |
| PS3 | 906 | T/C | T/C | T/C | T/C | T | T | T | T | T | T | T | T | T | T |
| PS4 | 913 | A | A | A | A | A | A | A | A | A | A | A | A/T | A | A |
| PS5 | 1077 | C | C | C | C | C | C | C | C | C | C | C | C | C | C |
| PS6 | 1468 | T | T | T/C | T | T | T/C | T | T | T | T | T | T | T | T |
| PS7 | 1474 | C | C | C | C | C | C | C/A | C | C | C | C | C | C | C |
| PS8 | 1610 | C/T | C/T | C | C/T | C | C | C/T | T | T | T | T | T | T | T |
| PS9 | 2422 | A | A | A | A | A | A | A | A | A | A | A | A | A | A |
| PS10 | 2738 | A | A | A | A | A | A | A | A | A/G | A | A | A | G | A/G |
| PS11 | 2789 | G | G | G | G | G | G | G | G | G | G | G/A | G | G | G/A |
| PS12 | 2934 | T | T | T/C | T | T | T/C | T | T | T | T | T | T | T | T |
| PS13 | 3000 | G | G | G | G | G | G | G | G | G | G | G/A | G | G | G/A |
| PS14 | 3044 | G | G | G | G | G | G | G | G | G | G | G | G | G | G |
| PS15 | 4552 | G/A | G | G | G/A | G | G | G | A | A | G/A | G/A | A | A | G/A |
| PS16 | 4822 | C | C | C | C | C | C | C | C | C | C | C | C | C | C |
| PS17 | 4999 | T | T | T | T | T | T | T | T | T | T | T | T | T | T |
| PS18 | 5077 | T/C | T | T | T/C | T | T | T | C | C | T/C | T/C | C | C | T/C |
| PS19 | 6535 | C | C | C | C | C | C | C | C | C | C | C | C | C | C |
| PS20 | 6625 | T | T | T/C | T | T | T/C | T | T | T | T | T | T | T | T |
| PS21 | 6650 | A | A | A | A/G | A | A | A | A | A/G | A | A | A | G | A/G |
| PS22 | 6714 | G | G | G | G | G | G | G | G | G | G | G | G | G | G |

(a)PS = polymorphic site;
(b)Position of PS in SEQ ID NO:1;
(c)Haplotype pairs are represented as 1^{st} haplotype/2^{nd} haplotype; with alleles of each haplotype shown 5′ to 3′ as 1^{st} polymorphism/2^{nd} polymorphism in each column.

3. A method for genotyping the Fc fragment of IgE, high affinity I, receptor for; alpha polypeptide (FCER1A) gene of an individual, comprising determining for the two copies of the FCER1A gene present in the individual the identity of the nucleotide pair at one or more polymorphic sites (PS) selected from the group consisting of PS1, PS2, PS3, PS4, PS5, PS6, PS7, PS8, PS9, PS10, PS11, PS12, PS13, PS14, PS15, PS16, PS17, PS18, PS19, PS20, PS21 and PS22, wherein the one or more polymorphic sites (PS) have the position and alternative alleles shown in SEQ ID NO:1.

4. The method of claim 3, wherein the determining step comprises:
(a) isolating from the individual a nucleic acid mixture comprising both copies of the FCER1A gene, or a fragment thereof, that are present in the individual;
(b) amplifying from the nucleic acid mixture a target region containing one of the selected polymorphic sites;
(c) hybridizing a primer extension oligonucleotide to one allele of the amplified target region, wherein the oligonucleotide is designed for genotyping the selected polymorphic site in the target region;
(d) performing a nucleic acid template-dependent, primer extension reaction on the hybridized oligonucleotide in the presence of at least one terminator of the reaction, wherein the terminator is complementary to one of the alternative nucleotides present at the selected polymorphic site; and
(e) detecting the presence and identity of the terminator in the extended oligonucleotide.

5. The method of claim 3, which comprises determining for the two copies of the FCER1A gene present in the individual the identity of the nucleotide pair at each of PS1-PS22.

6. A method for haplotyping the Fc fragment of IgE, high affinity I, receptor for; alpha polypeptide (FCER1A) gene of an individual which comprises determining, for one copy of the FCER1A gene present in the individual, the identity of the nucleotide at two or more polymorphic sites (PS) selected from the group consisting of PS1, PS2, PS3, PS4, PS5, PS6, PS7, PS8, PS9, PS10, PS11, PS12, PS13, PS14, PS15, PS16, PS17, PS18, PS19, PS20, PS21 and PS22, wherein the selected PS have the position and alternative alleles shown in SEQ ID NO:1.

7. The method of claim 6, wherein the determining step comprises:
(a) isolating from the individual a nucleic acid sample containing only one of the two copies of the FCER1A gene, or a fragment thereof, that is present in the individual;
(b) amplifying from the nucleic acid sample a target region containing one of the selected polymorphic sites;
(c) hybridizing a primer extension oligonucleotide to one allele of the amplified target region, wherein the oligonucleotide is designed for haplotyping the selected polymorphic site in the target region;
(d) performing a nucleic acid template-dependent, primer extension reaction on the hybridized oligonucleotide in the presence of at least one terminator of the reaction, wherein the terminator is complementary to one of the alternative nucleotides present at the selected polymorphic site; and
(e) detecting the presence and identity of the terminator in the extended oligonucleotide.

8. A method for predicting a haplotype pair for the Fc fragment of IgE, high affinity I, receptor for; alpha polypeptide (FCER1A) gene of an individual comprising:
(a) identifying a FCER1A genotype for the individual, wherein the genotype comprises the nucleotide pair at two or more polymorphic sites (PS) selected from the group consisting of PS1, PS2, PS3, PS4, PS5, PS6, PS7, PS8, PS9, PS10, PS11, PS12, PS13, PS14, PS15, PS16, PS17, PS18, PS19, PS20, PS21 and PS22, wherein the selected PS have the position and alternative alleles shown in SEQ ID NO:1;
(b) comparing the genotype to the haplotype pair data set forth in the table immediately below; and
(c) determining which haplotype pair is consistent with the genotype of the individual and with the haplotype pair data

| PS | PS | Haplotype Pair(c) Part 1 | | | | | | | | | | | | | | |
|---|---|---|---|---|---|---|---|---|---|---|---|---|---|---|---|---|
| Number(a) | Position(b) | 1/1 | 1/2 | 1/3 | 1/4 | 1/5 | 1/7 | 1/11 | 1/12 | 1/15 | 1/16 | 1/17 | 1/20 | 2/2 | 2/3 | 2/4 |
| | | | | | | | | | | | | | | | | |
|---|---|---|---|---|---|---|---|---|---|---|---|---|---|---|---|---|
| PS1 | 586 | T | T | T | T | T/G | T | T/G | T | T | T | T | T | T | T | T |
| PS2 | 657 | C | C/T | C/T | C | C | C | C | C | C | C | C | C/T | T | T | C/T |
| PS3 | 906 | T | T | T | T/C | T | T | T | T | T | T | T | T | T | T | T/C |
| PS4 | 913 | A | A | A | A | A | A | A | A | A | A | A | A | A | A | A |
| PS5 | 1077 | C | C | C | C | C | C | C | C | C | C | C | C/A | C | C | C |
| PS6 | 1468 | T | T | T | T | T | T | T/C | T | T | T | T | T | T | T | T |
| PS7 | 1474 | C | C | C | C | C | C | C | C | C/A | C | C | C | C | C | C |
| PS8 | 1610 | C | C | C | C | C | C/T | C | C | C/T | C | C/T | C/T | C | C | C |
| PS9 | 2422 | A | A | A | A | A | A | A | A | A | A | A | A | A | A | A |
| PS10 | 2738 | A | A | A | A | A | A/G | A | A | A | A | A/G | A | A | A | A |
| PS11 | 2789 | G | G | G | G | G | G | G | G | G | G | G | G | G | G | G |
| PS12 | 2934 | T | T | T | T | T | T | T/C | T | T | T | T | T | T | T | T |
| PS13 | 3000 | G | G | G | G | G | G | G | G | G | G | G | G | G | G | G |
| PS14 | 3044 | G | G | G | G | G | G | G | G | G | G/A | G | G | G | G | G |
| PS15 | 4552 | G | G | G | G | G | G/A | G | G | G | G | G | G/A | G | G | G |
| PS16 | 4822 | C | C | C | C | C | C | C | C | C | C | C | C | C | C | C |
| PS17 | 4999 | T | T/C | T/C | T | T | T | T | T | T | T | T | T/C | C | C | T/C |
| PS18 | 5077 | T | T | T | T | T | T/C | T | T | T | T | T/C | T/C | T | T | T |
| PS19 | 6535 | C | C | C | C | C | C | C | C/A | C | C/A | C | C | C | C | C |
| PS20 | 6625 | T | T | T | T | T | T | T/C | T | T | T | T | T | T | T | T |
| PS21 | 6650 | A | A | A | A | A | A/G | A | A | A | A | A | A | A | A | A |
| PS22 | 6714 | G | G | G/A | G | G | G | G | G | G | G | G | G | G | G/A | G |
| PS | PS | Haplotype Pair(c) Part 2 | | | | | | | | | | | | | | |
|---|---|---|---|---|---|---|---|---|---|---|---|---|---|---|---|---|
| Number(a) | Position(b) | 2/6 | 2/9 | 2/10 | 2/13 | 2/14 | 3/3 | 3/4 | 3/5 | 3/6 | 3/9 | 3/12 | 3/15 | 3/19 | 4/4 | 4/5 |
| | | | | | | | | | | | | | | | | |
|---|---|---|---|---|---|---|---|---|---|---|---|---|---|---|---|---|
| PS1 | 586 | T | T | T | T | T | T | T | T/G | T | T | T | T | T | T | T/G |
| PS2 | 657 | C/T | T | C/T | C/T | T | T | C/T | C/T | C/T | T | C/T | C/T | T | C | C |
| PS3 | 906 | T | T | T | T | T | T | T/C | T | T | T | T | T | T | C | T/C |
| PS4 | 913 | A | A | A | A | A | A | A | A | A | A | A | A | A | A | A |
| PS5 | 1077 | C | C | C | C | C | C | C | C | C | C | C | C | C | C | C |
| PS6 | 1468 | T | T | T | T | T | T | T | T | T | T | T | T | T | T | T |
| PS7 | 1474 | C | C | C | C | C | C | C | C | C | C | C | C/A | C | C | C |
| PS8 | 1610 | C/T | C | C/T | C/T | C/T | C | C | C | C/T | C | C | C/T | C | C | C |
| PS9 | 2422 | A | A/G | A | A | A | A | A | A | A | A/G | A | A | A | A | A |
| PS10 | 2738 | A | A | A | A | A | A | A | A | A | A | A | A | A | A | A |
| PS11 | 2789 | G | G | G/A | G | G | G | G | G | G | G | G | G | G | G | G |
| PS12 | 2934 | T | T | T | T | T | T | T | T | T | T | T | T | T | T | T |
| PS13 | 3000 | G | G | G/A | G | G | G | G | G | G | G | G | G | G | G | G |
| PS14 | 3044 | G | G | G | G | G | G | G | G | G | G | G | G | G | G | G |
| PS15 | 4552 | G/A | G | G | G/A | G | G | G | G | G/A | G | G | G | G | G | G |
| PS16 | 4822 | C | C | C | C | C | C | C | C | C | C | C | C | C/T | C | C |
| PS17 | 4999 | T/C | C | T/C | T/C | T/C | C | T/C | T/C | T/C | C | T/C | T/C | C | T | T |
| PS18 | 5077 | T/C | T | T | T/C | T | T | T | T | T/C | T | T | T | T | T | T |
| PS19 | 6535 | C | C | C | C | C | C | C | C | C | C | C/A | C | C | C | C |
| PS20 | 6625 | T | T | T | T | T | T | T | T | T | T | T | T | T | T | T |
| PS21 | 6650 | A | A | A | A/G | A | A | A | A | A | A | A | A | A | A | A |
| PS22 | 6714 | G | G | G | G | G/A | A | G/A | G/A | G/A | G/A | G/A | G/A | A | G | G |
| PS | PS | Haplotype Pair(c) Part 3 | | | | | | | | | | | | | |
|---|---|---|---|---|---|---|---|---|---|---|---|---|---|---|---|
| Number(a) | Position(b) | 4/6 | 4/8 | 4/11 | 4/13 | 5/5 | 5/11 | 5/15 | 6/6 | 6/7 | 6/8 | 6/10 | 6/18 | 7/7 | 7/10 |
| | | | | | | | | | | | | | | | |
|---|---|---|---|---|---|---|---|---|---|---|---|---|---|---|---|
| PS1 | 586 | T | T | T/G | T | G | G | T/G | T | T | T | T | T | T | T |
| PS2 | 657 | C | C | C | C | C | C | C | C | C | C | C | C | C | C |
| PS3 | 906 | T/C | T/C | T/C | T/C | T | T | T | T | T | T | T | T | T | T |
| PS4 | 913 | A | A | A | A | A | A | A | A | A | A | A | A/T | A | A |
| PS5 | 1077 | C | C | C | C | C | C | C | C | C | C | C | C | C | C |
| PS6 | 1468 | T | T | T/C | T | T | T/C | T | T | T | T | T | T | T | T |
| PS7 | 1474 | C | C | C | C | C | C | C/A | C | C | C | C | C | C | C |
| PS8 | 1610 | C/T | C/T | C | C/T | C | C | C/T | T | T | T | T | T | T | T |
| PS9 | 2422 | A | A | A | A | A | A | A | A | A | A | A | A | A | A |
| PS10 | 2738 | A | A | A | A | A | A | A | A | A/G | A | A | A | G | A/G |
| PS11 | 2789 | G | G | G | G | G | G | G | G | G | G | G/A | G | G | G/A |
| PS12 | 2934 | T | T | T/C | T | T | T/C | T | T | T | T | T | T | T | T |
| PS13 | 3000 | G | G | G | G | G | G | G | G | G | G | G/A | G | G | G/A |
| PS14 | 3044 | G | G | G | G | G | G | G | G | G | G | G | G | G | G |
| PS15 | 4552 | G/A | G | G | G/A | G | G | G | A | A | G/A | G/A | A | A | G/A |
| PS16 | 4822 | C | C | C | C | C | C | C | C | C | C | C | C | C | C |
| PS17 | 4999 | T | T | T | T | T | T | T | T | T | T | T | T | T | T |
| PS18 | 5077 | T/C | T | T | T/C | T | T | T | C | C | T/C | T/C | C | C | T/C |
| PS19 | 6535 | C | C | C | C | C | C | C | C | C | C | C | C | C | C |
| PS20 | 6625 | T | T | T/C | T | T | T/C | T | T | T | T | T | T | T | T |
| PS21 | 6650 | A | A | A | A/G | A | A | A | A | A/G | A | A | A | G | A/G |
| PS22 | 6714 | G | G | G | G | G | G | G | G | G | G | G | G | G | G |

(a)PS = polymorphic site;
(b)Position of PS in SEQ ID NO:1;
(c)Haplotype pairs are represented as 1^{st} haplotype/2^{nd} haplotype; with alleles of each haplotype shown 5′ to 3′ as 1^{st} polymorphism/2^{nd} polymorphism in each column.

9. The method of claim 8, wherein the identified genotype of the individual comprises the nucleotide pair at each of PS1-PS22, which have the position and alternative alleles shown in SEQ ID NO:1.

10. A method for identifying an association between a trait and at least one haplotype or haplotype pair of the Fc fragment of IgE, high affinity I, receptor for; alpha polypeptide (FCER1A) gene which comprises comparing the frequency of the haplotype or haplotype pair in a population exhibiting the trait with the frequency of the haplotype or haplotype pair in a reference population, wherein the haplotype is selected from haplotypes 1-20 shown in the table presented immediately below, wherein each of the haplotypes comprises a sequence of polymorphisms whose positions and identities are set forth in the table immediately below:
PS

| Num- | PS | Haplotype Number(c) | | | | | | | | | |
|---|---|---|---|---|---|---|---|---|---|---|---|
| ber(a) | Position(b) | 1 | 2 | 3 | 4 | 5 | 6 | 7 | 8 | 9 | 10 |
| | | | | | | | | | | | |
|---|---|---|---|---|---|---|---|---|---|---|---|
| PS1 | 586 | T | T | T | T | G | T | T | T | T | T |
| PS2 | 657 | C | T | T | C | C | C | C | C | T | C |
| PS3 | 906 | T | T | T | C | T | T | T | T | T | T |
| PS4 | 913 | A | A | A | A | A | A | A | A | A | A |
| PS5 | 1077 | C | C | C | C | C | C | C | C | C | C |
| PS6 | 1468 | T | T | T | T | T | T | T | T | T | T |
| PS7 | 1474 | C | C | C | C | C | C | C | C | C | C |
| PS8 | 1610 | C | C | C | C | C | T | T | T | C | T |
| PS9 | 2422 | A | A | A | A | A | A | A | A | G | A |
| PS10 | 2738 | A | A | A | A | A | A | G | A | A | A |
| PS11 | 2789 | G | G | G | G | G | G | G | G | G | A |
| PS12 | 2934 | T | T | T | T | T | T | T | T | T | T |
| PS13 | 3000 | G | G | G | G | G | G | G | G | G | A |
| PS14 | 3044 | G | G | G | G | G | G | G | G | G | G |
| PS15 | 4552 | G | G | G | G | G | A | A | G | G | G |
| PS16 | 4822 | C | C | C | C | C | C | C | C | C | C |
| PS17 | 4999 | T | C | C | T | T | T | T | T | C | T |
| PS18 | 5077 | T | T | T | T | T | C | C | T | T | T |
| PS19 | 6535 | C | C | C | C | C | C | C | C | C | C |
| PS20 | 6625 | T | T | T | T | T | T | T | T | T | T |
| PS21 | 6650 | A | A | A | A | A | A | G | A | A | A |
| PS22 | 6714 | G | G | A | G | G | G | G | G | G | G |

PS

| Num- | PS | Haplotype Number(c) | | | | | | | | | |
|---|---|---|---|---|---|---|---|---|---|---|---|
| ber(a) | Position(b) | 11 | 12 | 13 | 14 | 15 | 16 | 17 | 18 | 19 | 20 |
| | | | | | | | | | | | |
|---|---|---|---|---|---|---|---|---|---|---|---|
| PS1 | 586 | G | T | T | T | T | T | T | T | T | T |
| PS2 | 657 | C | C | C | T | C | C | C | C | T | T |
| PS3 | 906 | T | T | T | T | T | T | T | T | T | T |
| PS4 | 913 | A | A | A | A | A | A | A | T | A | A |
| PS5 | 1077 | C | C | C | C | C | C | C | C | C | A |
| PS6 | 1468 | C | T | T | T | T | T | T | T | T | T |
| PS7 | 1474 | C | C | C | C | A | C | C | C | C | C |
| PS8 | 1610 | C | C | T | T | T | C | T | T | C | T |
| PS9 | 2422 | A | A | A | A | A | A | A | A | A | A |
| PS10 | 2738 | A | A | A | A | A | A | G | A | A | A |
| PS11 | 2789 | G | G | G | G | G | G | G | G | G | G |
| PS12 | 2934 | C | T | T | T | T | T | T | T | T | T |
| PS13 | 3000 | G | G | G | G | G | G | G | G | G | G |
| PS14 | 3044 | G | G | G | G | G | A | G | G | G | G |
| PS15 | 4552 | G | G | A | G | G | G | G | A | G | A |
| PS16 | 4822 | C | C | C | C | C | C | C | C | T | C |
| PS17 | 4999 | T | T | T | C | T | T | T | T | C | C |
| PS18 | 5077 | T | T | C | T | T | T | T | C | T | C |
| PS19 | 6535 | C | A | C | C | C | A | C | C | C | C |
| PS20 | 6625 | C | T | T | T | T | T | T | T | T | T |
| PS21 | 6650 | A | A | G | A | A | A | A | A | A | A |
| PS22 | 6714 | G | G | G | A | G | G | G | G | A | G |

(a)PS = polymorphic site;
(b)Position of PS within SEQ ID NO:1;
(c)Alleles for haplotypes are presented 5′ to 3′ in each column;
and wherein the haplotype pair is selected from the haplotype pairs shown in the table immediately below, wherein each of the FCER1A haplotype pairs consists of first and second haplotypes which comprise first and second sequences of polymorphisms whose positions in SEQ ID NO:1 and identities are set forth in the table immediately below:

| PS | PS | Halotype Pair(c) Part 1 | | | | | | | | | | | | | | |
|---|---|---|---|---|---|---|---|---|---|---|---|---|---|---|---|---|
| Number(a) | Position(b) | 1/1 | 1/2 | 1/3 | 1/4 | 1/5 | 1/7 | 1/11 | 1/12 | 1/15 | 1/16 | 1/17 | 1/20 | 2/2 | 2/3 | 2/4 |
| | | | | | | | | | | | | | | | | |
|---|---|---|---|---|---|---|---|---|---|---|---|---|---|---|---|---|
| PS1 | 586 | T | T | T | T | T/G | T | T/G | T | T | T | T | T | T | T | T |
| PS2 | 657 | C | C/T | C/T | C | C | C | C | C | C | C | C | C/T | T | T | C/T |
| PS3 | 906 | T | T | T | T/C | T | T | T | T | T | T | T | T | T | T | T/C |
| PS4 | 913 | A | A | A | A | A | A | A | A | A | A | A | A | A | A | A |
| PS5 | 1077 | C | C | C | C | C | C | C | C | C | C | C | C/A | C | C | C |
| PS6 | 1468 | T | T | T | T | T | T | T/C | T | T | T | T | T | T | T | T |
| PS7 | 1474 | C | C | C | C | C | C | C | C | C/A | C | C | C | C | C | C |
| PS8 | 1610 | C | C | C | C | C | C/T | C | C | C/T | C | C/T | C/T | C | C | C |
| PS9 | 2422 | A | A | A | A | A | A | A | A | A | A | A | A | A | A | A |
| PS10 | 2738 | A | A | A | A | A | A/G | A | A | A | A | A/G | A | A | A | A |
| PS11 | 2789 | G | G | G | G | G | G | G | G | G | G | G | G | G | G | G |
| PS12 | 2934 | T | T | T | T | T | T | T/C | T | T | T | T | T | T | T | T |
| PS13 | 3000 | G | G | G | G | G | G | G | G | G | G | G | G | G | G | G |
| PS14 | 3044 | G | G | G | G | G | G | G | G | G | G/A | G | G | G | G | G |
| PS15 | 4552 | G | G | G | G | G | G/A | G | G | G | G | G | G/A | G | G | G |
| PS16 | 4822 | C | C | C | C | C | C | C | C | C | C | C | C | C | C | C |
| PS17 | 4999 | T | T/C | T/C | T | T | T | T | T | T | T | T | T/C | C | C | T/C |
| PS18 | 5077 | T | T | T | T | T | T/C | T | T | T | T | T/C | T/C | T | T | T |
| PS19 | 6535 | C | C | C | C | C | C | C | C/A | C | C/A | C | C | C | C | C |
| PS20 | 6625 | T | T | T | T | T | T | T/C | T | T | T | T | T | T | T | T |
| PS21 | 6650 | A | A | A | A | A | A/G | A | A | A | A | A | A | A | A | A |
| PS22 | 6714 | G | G | G/A | G | G | G | G | G | G | G | G | G | G | G/A | G |
| PS | PS | Haplotype Pair(c) Part 2 | | | | | | | | | | | | | | |
|---|---|---|---|---|---|---|---|---|---|---|---|---|---|---|---|---|
| Number(a) | Position(b) | 2/6 | 2/9 | 2/10 | 2/13 | 2/14 | 3/3 | 3/4 | 3/5 | 3/6 | 3/9 | 3/12 | 3/15 | 3/19 | 4/4 | 4/5 |
| | | | | | | | | | | | | | | | | |
|---|---|---|---|---|---|---|---|---|---|---|---|---|---|---|---|---|
| PS1 | 586 | T | T | T | T | T | T | T | T/G | T | T | T | T | T | T | T/G |
| PS2 | 657 | C/T | T | C/T | C/T | T | T | C/T | C/T | C/T | T | C/T | C/T | T | C | C |
| PS3 | 906 | T | T | T | T | T | T | T/C | T | T | T | T | T | T | C | T/C |
| PS4 | 913 | A | A | A | A | A | A | A | A | A | A | A | A | A | A | A |
| PS5 | 1077 | C | C | C | C | C | C | C | C | C | C | C | C | C | C | C |
| PS6 | 1468 | T | T | T | T | T | T | T | T | T | T | T | T | T | T | T |
| PS7 | 1474 | C | C | C | C | C | C | C | C | C | C | C | C/A | C | C | C |
| PS8 | 1610 | C/T | C | C/T | C/T | C/T | C | C | C | C/T | C | C | C/T | C | C | C |
| PS9 | 2422 | A | A/G | A | A | A | A | A | A | A | A/G | A | A | A | A | A |
| PS10 | 2738 | A | A | A | A | A | A | A | A | A | A | A | A | A | A | A |
| PS11 | 2789 | G | G | G/A | G | G | G | G | G | G | G | G | G | G | G | G |
| PS12 | 2934 | T | T | T | T | T | T | T | T | T | T | T | T | T | T | T |
| PS13 | 3000 | G | G | G/A | G | G | G | G | G | G | G | G | G | G | G | G |
| PS14 | 3044 | G | G | G | G | G | G | G | G | G | G | G | G | G | G | G |
| PS15 | 4552 | G/A | G | G | G/A | G | G | G | G | G/A | G | G | G | G | G | G |
| PS16 | 4822 | C | C | C | C | C | C | C | C | C | C | C | C | C/T | C | C |
| PS17 | 4999 | T/C | C | T/C | T/C | T/C | C | T/C | T/C | T/C | C | T/C | T/C | C | T | T |
| PS18 | 5077 | T/C | T | T | T/C | T | T | T | T | T/C | T | T | T | T | T | T |
| PS19 | 6535 | C | C | C | C | C | C | C | C | C | C | C/A | C | C | C | C |
| PS20 | 6625 | T | T | T | T | T | T | T | T | T | T | T | T | T | T | T |
| PS21 | 6650 | A | A | A | A/G | A | A | A | A | A | A | A | A | A | A | A |
| PS22 | 6714 | G | G | G | G | G/A | A | G/A | G/A | G/A | G/A | G/A | G/A | A | G | G |
| PS | PS | Haplotype Pair(c) Part 3 | | | | | | | | | | | | | |
|---|---|---|---|---|---|---|---|---|---|---|---|---|---|---|---|
| Number(a) | Position(b) | 4/6 | 4/8 | 4/11 | 4/13 | 5/5 | 5/11 | 5/15 | 6/6 | 6/7 | 6/8 | 6/10 | 6/18 | 7/7 | 7/10 |
| | | | | | | | | | | | | | | | |
|---|---|---|---|---|---|---|---|---|---|---|---|---|---|---|---|
| PS1 | 586 | T | T | T/G | T | G | G | T/G | T | T | T | T | T | T | T |
| PS2 | 657 | C | C | C | C | C | C | C | C | C | C | C | C | C | C |
| PS3 | 906 | T/C | T/C | T/C | T/C | T | T | T | T | T | T | T | T | T | T |
| PS4 | 913 | A | A | A | A | A | A | A | A | A | A | A | A/T | A | A |
| PS5 | 1077 | C | C | C | C | C | C | C | C | C | C | C | C | C | C |
| PS6 | 1468 | T | T | T/C | T | T | T/C | T | T | T | T | T | T | T | T |
| PS7 | 1474 | C | C | C | C | C | C | C/A | C | C | C | C | C | C | C |
| PS8 | 1610 | C/T | C/T | C | C/T | C | C | C/T | T | T | T | T | T | T | T |
| PS9 | 2422 | A | A | A | A | A | A | A | A | A | A | A | A | A | A |
| PS10 | 2738 | A | A | A | A | A | A | A | A | A/G | A | A | A | G | A/G |
| PS11 | 2789 | G | G | G | G | G | G | G | G | G | G | G/A | G | G | G/A |
| PS12 | 2934 | T | T | T/C | T | T | T/C | T | T | T | T | T | T | T | T |
| PS13 | 3000 | G | G | G | G | G | G | G | G | G | G | G/A | G | G | G/A |
| PS14 | 3044 | G | G | G | G | G | G | G | G | G | G | G | G | G | G |
| PS15 | 4552 | G/A | G | G | G/A | G | G | G | A | A | G/A | G/A | A | A | G/A |
| PS16 | 4822 | C | C | C | C | C | C | C | C | C | C | C | C | C | C |
| PS17 | 4999 | T | T | T | T | T | T | T | T | T | T | T | T | T | T |
| PS18 | 5077 | T/C | T | T | T/C | T | T | T | C | C | T/C | T/C | C | C | T/C |
| PS19 | 6535 | C | C | C | C | C | C | C | C | C | C | C | C | C | C |
| PS20 | 6625 | T | T | T/C | T | T | T/C | T | T | T | T | T | T | T | T |
| PS21 | 6650 | A | A | A | A/G | A | A | A | A | A/G | A | A | A | G | A/G |
| PS22 | 6714 | G | G | G | G | G | G | G | G | G | G | G | G | G | G |

(a)PS = polymorphic site;
(b)Position of PS in SEQ ID NO:1;
(c)Haplotype pairs are represented as 1^{st} haplotype/2^{nd} haplotype; with alleles of each haplotype shown 5′ to 3′ as 1^{st} polymorphism/2^{nd} polymorphism in each column;
wherein a higher frequency of the haplotype or haplotype pair in the trait population than in the reference population indicates the trait is associated with the haplotype or haplotype pair.

11. The method of claim 10, wherein the trait is a clinical response to a drug targeting FCER1A or to a drug for treating a condition or disease predicted to be associated with FCER1A activity.

12. An isolated oligonucleotide designed for detecting a polymorphism in the Fc fragment of IgE, high affinity I, receptor for; alpha polypeptide (FCER1A) gene at a polymorphic site (PS) selected from the group consisting of PS1, PS2, PS3, PS4, PS5, PS6, PS7, PS8, PS9, PS10, PS11, PS12, PS13, PS14, PS15, PS16, PS17, PS18, PS19, PS20, PS21 and PS22, wherein the selected PS have the position and alternative alleles shown in SEQ ID NO:1.

13. The isolated oligonucleotide of claim 12, which is an allele-specific oligonucleotide that specifically hybridizes to an allele of the FCER1A gene at a region containing the polymorphic site.

14. The allele-specific oligonucleotide of claim 13, which comprises a nucleotide sequence selected from the group consisting of SEQ ID NOS:4-25, the complements of SEQ ID NOS:4-25, and SEQ ID NOS:26-69.

15. The isolated oligonucleotide of claim 12, which is a primer-extension oligonucleotide.

16. The primer-extension oligonucleotide of claim 15, which comprises a nucleotide sequence selected from the group consisting of SEQ ID NOS:70-113.

17. A kit for haplotyping or genotyping the Fc fragment of IgE, high affinity I, receptor for; alpha polypeptide (FCER1A) gene of an individual, which comprises a set of oligonucleotides designed to haplotype or genotype each of polymorphic sites (PS) PS1, PS2, PS3, PS4, PS5, PS6, PS7, PS8, PS9, PS10, PS11, PS12, PS13, PS14, PS15, PS16, PS17, PS18, PS19, PS20, PS21 and PS22, wherein the selected PS have the position and alternative alleles shown in SEQ ID NO:1.

18. An isolated polynucleotide comprising a nucleotide sequence selected from the group consisting of:
(a) a first nucleotide sequence which comprises a Fc fragment of IgE, high affinity I, receptor for; alpha polypeptide (FCER1A) isogene, wherein the FCER1A isogene is selected from the group consisting of isogenes 1 and 3-20 shown in the table immediately below and wherein each of the isogenes comprises the regions of SEQ ID NO:1 shown in the table immediately below and wherein each of the isogenes 1 and 3-20 is further defined by the corresponding sequence of polymorphisms whose positions and identities are set forth in the table immediately below; and

| Regions | PS | PS | Isogene Number(d) (Part 1) | | | | | | | | |
|---|---|---|---|---|---|---|---|---|---|---|---|
| Examined(a) | Number(b) | Position(c) | 1 | 3 | 4 | 5 | 6 | 7 | 8 | 9 | 10 |
| | | | | | | | | | | | |
|---|---|---|---|---|---|---|---|---|---|---|---|
| 319-1709 | PS1 | 586 | T | T | T | G | T | T | T | T | T |
| 319-1709 | PS2 | 657 | C | T | C | C | C | C | C | T | C |
| 319-1709 | PS3 | 906 | T | T | C | T | T | T | T | T | T |
| 319-1709 | PS4 | 913 | A | A | A | A | A | A | A | A | A |
| 319-1709 | PS5 | 1077 | C | C | C | C | C | C | C | C | C |
| 319-1709 | PS6 | 1468 | T | T | T | T | T | T | T | T | T |
| 319-1709 | PS7 | 1474 | C | C | C | C | C | C | C | C | C |
| 319-1709 | PS8 | 1610 | C | C | C | C | T | T | T | C | T |
| 2351-3067 | PS9 | 2422 | A | A | A | A | A | A | A | G | A |
| 2351-3067 | PS10 | 2738 | A | A | A | A | A | G | A | A | A |
| 2351-3067 | PS11 | 2789 | G | G | G | G | G | G | G | G | A |
| 2351-3067 | PS12 | 2934 | T | T | T | T | T | T | T | T | T |
| 2351-3067 | PS13 | 3000 | G | G | G | G | G | G | G | G | A |
| 2351-3067 | PS14 | 3044 | G | G | G | G | G | G | G | G | G |
| 4359-5177 | PS15 | 4552 | G | G | G | G | A | A | G | G | G |
| 4359-5177 | PS16 | 4822 | C | C | C | C | C | C | C | C | C |
| 4359-5177 | PS17 | 4999 | T | C | T | T | T | T | T | C | T |
| 4359-5177 | PS18 | 5077 | T | T | T | T | C | C | T | T | T |
| 6200-7073 | PS19 | 6535 | C | C | C | C | C | C | C | C | C |
| 6200-7073 | PS20 | 6625 | T | T | T | T | T | T | T | T | T |
| 6200-7073 | PS21 | 6650 | A | A | A | A | A | G | A | A | A |
| 6200-7073 | PS22 | 6714 | G | A | G | G | G | G | G | G | G |
| Regions | PS | PS | Isogene Number(d) (Part 2) | | | | | | | | | |
|---|---|---|---|---|---|---|---|---|---|---|---|---|
| Examined(a) | Number(b) | Position(c) | 11 | 12 | 13 | 14 | 15 | 16 | 17 | 18 | 19 | 20 |
| | | | | | | | | | | | | |
|---|---|---|---|---|---|---|---|---|---|---|---|---|
| 319-1709 | PS1 | 586 | G | T | T | T | T | T | T | T | T | T |
| 319-1709 | PS2 | 657 | C | C | C | T | C | C | C | C | T | T |
| 319-1709 | PS3 | 906 | T | T | T | T | T | T | T | T | T | T |
| 319-1709 | PS4 | 913 | A | A | A | A | A | A | A | T | A | A |
| 319-1709 | PS5 | 1077 | C | C | C | C | C | C | C | C | C | A |
| 319-1709 | PS6 | 1468 | C | T | T | T | T | T | T | T | T | T |
| 319-1709 | PS7 | 1474 | C | C | C | C | A | C | C | C | C | C |
| 319-1709 | PS8 | 1610 | C | C | T | T | T | C | T | T | C | T |
| 2351-3067 | PS9 | 2422 | A | A | A | A | A | A | A | A | A | A |
| 2351-3067 | PS10 | 2738 | A | A | A | A | A | A | G | A | A | A |
| 2351-3067 | PS11 | 2789 | G | G | G | G | G | G | G | G | G | G |
| 2351-3067 | PS12 | 2934 | C | T | T | T | T | T | T | T | T | T |
| 2351-3067 | PS13 | 3000 | G | G | G | G | G | G | G | G | G | G |
| 2351-3067 | PS14 | 3044 | G | G | G | G | G | A | G | G | G | G |
| 4359-5177 | PS15 | 4552 | G | G | A | G | G | G | G | A | G | A |
| 4359-5177 | PS16 | 4822 | C | C | C | C | C | C | C | C | T | C |
| 4359-5177 | PS17 | 4999 | T | T | T | C | T | T | T | T | C | C |
| 4359-5177 | PS18 | 5077 | T | T | C | T | T | T | T | C | T | C |
| 6200-7073 | PS19 | 6535 | C | A | C | C | C | A | C | C | C | C |
| 6200-7073 | PS20 | 6625 | C | T | T | T | T | T | T | T | T | T |
| 6200-7073 | PS21 | 6650 | A | A | G | A | A | A | A | A | A | A |
| 6200-7073 | PS22 | 6714 | G | G | G | A | G | G | G | G | A | G |

(a)Region examined represents the nucleotide positions defining the start and stop positions within the 1^{st} SEQ ID NO of the sequenced region;
(b)PS = polymorphic site;
(c)Position of PS in SEQ ID NO:1;
(d)Alleles for isogenes are presented 5′ to 3′ in each column;
(b) a second nucleotide sequence which is complementary to the first nucleotide sequence.

19. The isolated polynucleotide of claim 18, which is a DNA molecule and comprises both the first and second nucleotide sequences and further comprises expression regulatory elements operably linked to the first nucleotide sequence.

20. A recombinant nonhuman organism transformed or transfected with the isolated polynucleotide of claim 19, wherein the organism expresses a FCER1A protein that is encoded by the first nucleotide sequence.

21. The recombinant nonhuman organism of claim 20, which is a transgenic animal.

22. An isolated fragment of a Fc fragment of IgE, high affinity I, receptor for; alpha polypeptide (FCER1A) isogene, wherein the fragment comprises at least 10 nucleotides in one of the regions of SEQ ID NO:1 shown in the table immediately below and wherein the fragment comprises one or more polymorphisms selected from the group consisting of guanine at PS1, cytosine at PS2, cytosine at PS3, thymine at PS4, adenine at PS5, cytosine at PS6, adenine at PS7, thymine at PS8, guanine at PS9, guanine at PS10, adenine at PS11, cytosine at PS12, adenine at PS13, adenine at PS14, adenine at PS15, thymine at PS16, thymine at PS17, cytosine at PS18, adenine at PS19, cytosine at PS20, guanine at PS21 and adenine at PS22, wherein the selected polymorphism has the position set forth in the table immediately below:

| Regions | PS | PS | Isogene Number(d) (Part 1) | | | | | | | | |
|---|---|---|---|---|---|---|---|---|---|---|---|
| Examined(a) | Number(b) | Position(c) | 1 | 3 | 4 | 5 | 6 | 7 | 8 | 9 | 10 |
| | | | | | | | | | | | |
|---|---|---|---|---|---|---|---|---|---|---|---|
| 319-1709 | PS1 | 586 | T | T | T | G | T | T | T | T | T |
| 319-1709 | PS2 | 657 | C | T | C | C | C | C | C | T | C |
| 319-1709 | PS3 | 906 | T | T | C | T | T | T | T | T | T |
| 319-1709 | PS4 | 913 | A | A | A | A | A | A | A | A | A |
| 319-1709 | PS5 | 1077 | C | C | C | C | C | C | C | C | C |
| 319-1709 | PS6 | 1468 | T | T | T | T | T | T | T | T | T |
| 319-1709 | PS7 | 1474 | C | C | C | C | C | C | C | C | C |
| 319-1709 | PS8 | 1610 | C | C | C | C | T | T | T | C | T |
| 2351-3067 | PS9 | 2422 | A | A | A | A | A | A | A | G | A |
| 2351-3067 | PS10 | 2738 | A | A | A | A | A | G | A | A | A |
| 2351-3067 | PS11 | 2789 | G | G | G | G | G | G | G | G | A |
| 2351-3067 | PS12 | 2934 | T | T | T | T | T | T | T | T | T |
| 2351-3067 | PS13 | 3000 | G | G | G | G | G | G | G | G | A |
| 2351-3067 | PS14 | 3044 | G | G | G | G | G | G | G | G | G |
| 4359-5177 | PS15 | 4552 | G | G | G | G | A | A | G | G | G |
| 4359-5177 | PS16 | 4822 | C | C | C | C | C | C | C | C | C |
| 4359-5177 | PS17 | 4999 | T | C | T | T | T | T | T | C | T |
| 4359-5177 | PS18 | 5077 | T | T | T | T | C | C | T | T | T |
| 6200-7073 | PS19 | 6535 | C | C | C | C | C | C | C | C | C |
| 6200-7073 | PS20 | 6625 | T | T | T | T | T | T | T | T | T |
| 6200-7073 | PS21 | 6650 | A | A | A | A | A | G | A | A | A |
| 6200-7073 | PS22 | 6714 | G | A | G | G | G | G | G | G | G |
| Regions | PS | PS | Isogene Number(d) (Part 2) | | | | | | | | | |
|---|---|---|---|---|---|---|---|---|---|---|---|---|
| Examined(a) | Number(b) | Position(c) | 11 | 12 | 13 | 14 | 15 | 16 | 17 | 18 | 19 | 20 |
| | | | | | | | | | | | | |
|---|---|---|---|---|---|---|---|---|---|---|---|---|
| 319-1709 | PS1 | 586 | G | T | T | T | T | T | T | T | T | T |
| 319-1709 | PS2 | 657 | C | C | C | T | C | C | C | C | T | T |
| 319-1709 | PS3 | 906 | T | T | T | T | T | T | T | T | T | T |
| 319-1709 | PS4 | 913 | A | A | A | A | A | A | A | T | A | A |
| 319-1709 | PS5 | 1077 | C | C | C | C | C | C | C | C | C | A |
| 319-1709 | PS6 | 1468 | C | T | T | T | T | T | T | T | T | T |
| 319-1709 | PS7 | 1474 | C | C | C | C | A | C | C | C | C | C |
| 319-1709 | PS8 | 1610 | C | C | T | T | T | C | T | T | C | T |
| 2351-3067 | PS9 | 2422 | A | A | A | A | A | A | A | A | A | A |
| 2351-3067 | PS10 | 2738 | A | A | A | A | A | A | G | A | A | A |
| 2351-3067 | PS11 | 2789 | G | G | G | G | G | G | G | G | G | G |
| 2351-3067 | PS12 | 2934 | C | T | T | T | T | T | T | T | T | T |
| 2351-3067 | PS13 | 3000 | G | G | G | G | G | G | G | G | G | G |
| 2351-3067 | PS14 | 3044 | G | G | G | G | G | A | G | G | G | G |
| 4359-5177 | PS15 | 4552 | G | G | A | G | G | G | G | A | G | A |
| 4359-5177 | PS16 | 4822 | C | C | C | C | C | C | C | C | T | C |
| 4359-5177 | PS17 | 4999 | T | T | T | C | T | T | T | T | C | C |
| 4359-5177 | PS18 | 5077 | T | T | C | T | T | T | T | C | T | C |
| 6200-7073 | PS19 | 6535 | C | A | C | C | C | A | C | C | C | C |
| 6200-7073 | PS20 | 6625 | C | T | T | T | T | T | T | T | T | T |
| 6200-7073 | PS21 | 6650 | A | A | G | A | A | A | A | A | A | A |
| 6200-7073 | PS22 | 6714 | G | G | G | A | G | G | G | G | A | G |

(a)Region examined represents the nucleotide positions defining the start and stop positions within SEQ ID NO: 1 of the regions sequenced;
(b)PS = polymorphic site;
(c)Position of PS within SEQ ID NO:1;
(d)Alleles for FCER1A isogenes are presented 5′ to 3′ in each column.

23. An isolated polynucleotide comprising a coding sequence for a FCER1A isogene, wherein the coding sequence comprises the regions of SEQ ID NO:2, except at each of the polymorphic sites which have the positions in SEQ ID NO:2 and polymorphisms set forth in the table immediately below:

| | | | |
|---|---|---|---|
| | | | Isogene Coding |
| Regions | PS | Position | Sequence Number(d) |
| | | | | | | | | |
|---|---|---|---|---|---|---|---|---|
| Examined(a) | Number(b) | (c) | 7 | 10 | 12 | 16 | 17 | 19 |
| | | | | | | | | |
|---|---|---|---|---|---|---|---|---|
| 1-774 | PS10 | 251 | G | A | A | A | G | A |
| 1-774 | PS11 | 302 | G | A | G | G | G | G |
| 1-774 | PS16 | 503 | C | C | C | C | C | T |
| 1-774 | PS19 | 741 | C | C | A | A | C | C |

(a)Region examined represents the nucleotide positions defining the start and stop positions within SEQ ID NO:2 of the regions sequenced;
(b)PS = polymorphic site;
(c)Position of PS within SEQ ID NO:2;
(d)Alleles for FCER1A isogenes are presented 5′ to 3′ in each column.

24. A recombinant nonhuman organism transformed or transfected with the isolated polynucleotide of claim 23, wherein the organism expresses a Fc fragment of IgE, high affinity I, receptor for; alpha polypeptide (FCER1A) protein that is encoded by the polymorphic variant sequence.

25. The recombinant nonhuman organism of claim 24, which is a transgenic animal.

26. An isolated fragment of a FCER1A coding sequence, wherein the fragment comprises one or more polymorphisms selected from the group consisting of guanine at a position corresponding to nucleotide 251, adenine at a position corresponding to nucleotide 302, thymine at a position corresponding to nucleotide 530 and adenine at a position corresponding to nucleotide 741 in SEQ ID NO:2.

27. An isolated polypeptide comprising an amino acid sequence which is a polymorphic variant of a reference sequence for the Fc fragment of IgE, high affinity I, receptor for; alpha polypeptide (FCER1A) protein, wherein the reference sequence comprises SEQ ID NO:3, except the polymorphic variant comprises one or more variant amino acids selected from the group consisting of arginine at a position corresponding to amino acid position 84, asparagine at a position corresponding to amino acid position 101, methionine at a position corresponding to amino acid position 177 and lysine at a position corresponding to amino acid position 247.

28. An isolated monoclonal antibody specific for and immunoreactive with the isolated polypeptide of claim 27.

29. A method for screening for drugs targeting the isolated polypeptide of claim 27 which comprises contacting the FCER1A polymorphic variant with a candidate agent and assaying for binding activity.

30. An isolated fragment of a FCER1A protein, wherein the fragment comprises one or more variant amino acids selected from the group consisting of arginine at a position corresponding to amino acid position 84, asparagine at a position corresponding to amino acid position 101, methionine at a position corresponding to amino acid position 177 and lysine at a position corresponding to amino acid position 247 in SEQ ID NO:3.

31. A computer system for storing and analyzing polymorphism data for the Fc fragment of IgE, high affinity I, receptor for; alpha polypeptide gene, comprising:
(a) a central processing unit (CPI);
(b) a communication interface;
(c) a display device;
(d) an input device; and
(e) a database containing the polymorphism data;
wherein the polymorphism data comprises any one or more of the haplotypes set forth in the table immediately below:

| | | |
|---|---|---|
| PS | | |
| Num- | PS | Haplotype Number(c) |
| | | | | | | | | | | | |
|---|---|---|---|---|---|---|---|---|---|---|---|
| ber(a) | Position(b) | 1 | 2 | 3 | 4 | 5 | 6 | 7 | 8 | 9 | 10 |
| | | | | | | | | | | | |
|---|---|---|---|---|---|---|---|---|---|---|---|
| PS1 | 586 | T | T | T | T | G | T | T | T | T | T |
| PS2 | 657 | C | T | T | C | C | C | C | C | T | C |
| PS3 | 906 | T | T | T | C | T | T | T | T | T | T |
| PS4 | 913 | A | A | A | A | A | A | A | A | A | A |
| PS5 | 1077 | C | C | C | C | C | C | C | C | C | C |
| PS6 | 1468 | T | T | T | T | T | T | T | T | T | T |
| PS7 | 1474 | C | C | C | C | C | C | C | C | C | C |
| PS8 | 1610 | C | C | C | C | C | T | T | T | C | T |
| PS9 | 2422 | A | A | A | A | A | A | A | A | G | A |
| PS10 | 2738 | A | A | A | A | A | A | G | A | A | A |
| PS11 | 2789 | G | G | G | G | G | G | G | G | G | A |
| PS12 | 2934 | T | T | T | T | T | T | T | T | T | T |
| PS13 | 3000 | G | G | G | G | G | G | G | G | G | A |
| PS14 | 3044 | G | G | G | G | G | G | G | G | G | G |
| PS15 | 4552 | G | G | G | G | G | A | A | G | G | G |
| PS16 | 4822 | C | C | C | C | C | C | C | C | C | C |
| PS17 | 4999 | T | C | C | T | T | T | T | T | C | T |
| PS18 | 5077 | T | T | T | T | T | C | C | T | T | T |
| PS19 | 6535 | C | C | C | C | C | C | C | C | C | C |
| PS20 | 6625 | T | T | T | T | T | T | T | T | T | T |
| PS21 | 6650 | A | A | A | A | A | A | G | A | A | A |
| PS22 | 6714 | G | G | A | G | G | G | G | G | G | G |
| | | |
|---|---|---|
| PS | | |
| Num- | PS | Haplotype Number(c) |
| | | | | | | | | | | | |
|---|---|---|---|---|---|---|---|---|---|---|---|
| ber(a) | Position(b) | 11 | 12 | 13 | 14 | 15 | 16 | 17 | 18 | 19 | 20 |
| | | | | | | | | | | | |
|---|---|---|---|---|---|---|---|---|---|---|---|
| PS1 | 586 | G | T | T | T | T | T | T | T | T | T |
| PS2 | 657 | C | C | C | T | C | C | C | C | T | T |
| PS3 | 906 | T | T | T | T | T | T | T | T | T | T |
| PS4 | 913 | A | A | A | A | A | A | A | T | A | A |
| PS5 | 1077 | C | C | C | C | C | C | C | C | C | A |
| PS6 | 1468 | C | T | T | T | T | T | T | T | T | T |
| PS7 | 1474 | C | C | C | C | A | C | C | C | C | C |
| PS8 | 1610 | C | C | T | T | T | C | T | T | C | T |
| PS9 | 2422 | A | A | A | A | A | A | A | A | A | A |
| PS10 | 2738 | A | A | A | A | A | A | G | A | A | A |
| PS11 | 2789 | G | G | G | G | G | G | G | G | G | G |
| PS12 | 2934 | C | T | T | T | T | T | T | T | T | T |
| PS13 | 3000 | G | G | G | G | G | G | G | G | G | G |
| PS14 | 3044 | G | G | G | G | G | A | G | G | G | G |
| PS15 | 4552 | G | G | A | G | G | G | G | A | G | A |
| PS16 | 4822 | C | C | C | C | C | C | C | C | T | C |
| PS17 | 4999 | T | T | T | C | T | T | T | T | C | C |
| PS18 | 5077 | T | T | C | T | T | T | T | C | T | C |
| PS19 | 6535 | C | A | C | C | C | A | C | C | C | C |
| PS20 | 6625 | C | T | T | T | T | T | T | T | T | T |
| PS21 | 6650 | A | A | G | A | A | A | A | A | A | A |
| PS22 | 6714 | G | G | G | A | G | G | G | G | A | G |

(a)PS = polymorphic site;
(b)Position of PS within SEQ ID NO:1;
(c)Alleles for haplotypes are presented 5′ to 3′ in each column;
the haplotype pairs set forth in the table immediately below:

| PS | PS | Haplotype Pair(c) Part 1 | | | | | | | | | | | | | | |
|---|---|---|---|---|---|---|---|---|---|---|---|---|---|---|---|---|
| Number(a) | Position(b) | 1/1 | 1/2 | 1/3 | 1/4 | 1/5 | 1/7 | 1/11 | 1/12 | 1/15 | 1/16 | 1/17 | 1/20 | 2/2 | 2/3 | 2/4 |
| | | | | | | | | | | | | | | | | |
|---|---|---|---|---|---|---|---|---|---|---|---|---|---|---|---|---|
| PS1 | 586 | T | T | T | T | T/G | T | T/G | T | T | T | T | T | T | T | T |
| PS2 | 657 | C | C/T | C/T | C | C | C | C | C | C | C | C | C/T | T | T | C/T |
| PS3 | 906 | T | T | T | T/C | T | T | T | T | T | T | T | T | T | T | T/C |
| PS4 | 913 | A | A | A | A | A | A | A | A | A | A | A | A | A | A | A |
| PS5 | 1077 | C | C | C | C | C | C | C | C | C | C | C | C/A | C | C | C |
| PS6 | 1468 | T | T | T | T | T | T | T/C | T | T | T | T | T | T | T | T |
| PS7 | 1474 | C | C | C | C | C | C | C | C | C/A | C | C | C | C | C | C |
| PS8 | 1610 | C | C | C | C | C | C/T | C | C | C/T | C | C/T | C/T | C | C | C |
| PS9 | 2422 | A | A | A | A | A | A | A | A | A | A | A | A | A | A | A |
| PS10 | 2738 | A | A | A | A | A | A/G | A | A | A | A | A/G | A | A | A | A |
| PS11 | 2789 | G | G | G | G | G | G | G | G | G | G | G | G | G | G | G |
| PS12 | 2934 | T | T | T | T | T | T | T/C | T | T | T | T | T | T | T | T |
| PS13 | 3000 | G | G | G | G | G | G | G | G | G | G | G | G | G | G | G |
| PS14 | 3044 | G | G | G | G | G | G | G | G | G | G/A | G | G | G | G | G |
| PS15 | 4552 | G | G | G | G | G | G/A | G | G | G | G | G | G/A | G | G | G |
| PS16 | 4822 | C | C | C | C | C | C | C | C | C | C | C | C | C | C | C |
| PS17 | 4999 | T | T/C | T/C | T | T | T | T | T | T | T | T | T/C | C | C | T/C |
| PS18 | 5077 | T | T | T | T | T | T/C | T | T | T | T | T/C | T/C | T | T | T |
| PS19 | 6535 | C | C | C | C | C | C | C | C/A | C | C/A | C | C | C | C | C |
| PS20 | 6625 | T | T | T | T | T | T | T/C | T | T | T | T | T | T | T | T |
| PS21 | 6650 | A | A | A | A | A | A/G | A | A | A | A | A | A | A | A | A |
| PS22 | 6714 | G | G | G/A | G | G | G | G | G | G | G | G | G | G | G/A | G |
| PS | PS | Haplotype Pair(c) Part 2 | | | | | | | | | | | | | | |
|---|---|---|---|---|---|---|---|---|---|---|---|---|---|---|---|---|
| Number(a) | Position(b) | 2/6 | 2/9 | 2/10 | 2/13 | 2/14 | 3/3 | 3/4 | 3/5 | 3/6 | 3/9 | 3/12 | 3/15 | 3/19 | 4/4 | 4/5 |
| | | | | | | | | | | | | | | | | |
|---|---|---|---|---|---|---|---|---|---|---|---|---|---|---|---|---|
| PS1 | 586 | T | T | T | T | T | T | T | T/G | T | T | T | T | T | T | T/G |
| PS2 | 657 | C/T | T | C/T | C/T | T | T | C/T | C/T | C/T | T | C/T | C/T | T | C | C |
| PS3 | 906 | T | T | T | T | T | T | T/C | T | T | T | T | T | T | C | T/C |
| PS4 | 913 | A | A | A | A | A | A | A | A | A | A | A | A | A | A | A |
| PS5 | 1077 | C | C | C | C | C | C | C | C | C | C | C | C | C | C | C |
| PS6 | 1468 | T | T | T | T | T | T | T | T | T | T | T | T | T | T | T |
| PS7 | 1474 | C | C | C | C | C | C | C | C | C | C | C | C/A | C | C | C |
| PS8 | 1610 | C/T | C | C/T | C/T | C/T | C | C | C | C/T | C | C | C/T | C | C | C |
| PS9 | 2422 | A | A/G | A | A | A | A | A | A | A | A/G | A | A | A | A | A |
| PS10 | 2738 | A | A | A | A | A | A | A | A | A | A | A | A | A | A | A |
| PS11 | 2789 | G | G | G/A | G | G | G | G | G | G | G | G | G | G | G | G |
| PS12 | 2934 | T | T | T | T | T | T | T | T | T | T | T | T | T | T | T |
| PS13 | 3000 | G | G | G/A | G | G | G | G | G | G | G | G | G | G | G | G |
| PS14 | 3044 | G | G | G | G | G | G | G | G | G | G | G | G | G | G | G |
| PS15 | 4552 | G/A | G | G | G/A | G | G | G | G | G/A | G | G | G | G | G | G |
| PS16 | 4822 | C | C | C | C | C | C | C | C | C | C | C | C | C/T | C | C |
| PS17 | 4999 | T/C | C | T/C | T/C | T/C | C | T/C | T/C | T/C | C | T/C | T/C | C | T | T |
| PS18 | 5077 | T/C | T | T | T/C | T | T | T | T | T/C | T | T | T | T | T | T |
| PS19 | 6535 | C | C | C | C | C | C | C | C | C | C | C/A | C | C | C | C |
| PS20 | 6625 | T | T | T | T | T | T | T | T | T | T | T | T | T | T | T |
| PS21 | 6650 | A | A | A | A/G | A | A | A | A | A | A | A | A | A | A | A |
| PS22 | 6714 | G | G | G | G | G/A | A | G/A | G/A | G/A | G/A | G/A | G/A | A | G | G |
| PS | PS | Haplotype Pair(c) Part 3 | | | | | | | | | | | | | |
|---|---|---|---|---|---|---|---|---|---|---|---|---|---|---|---|
| Number(a) | Position(b) | 4/6 | 4/8 | 4/11 | 4/13 | 5/5 | 5/11 | 5/15 | 6/6 | 6/7 | 6/8 | 6/10 | 6/18 | 7/7 | 7/10 |
| | | | | | | | | | | | | | | | |
|---|---|---|---|---|---|---|---|---|---|---|---|---|---|---|---|
| PS1 | 586 | T | T | T/G | T | G | G | T/G | T | T | T | T | T | T | T |
| PS2 | 657 | C | C | C | C | C | C | C | C | C | C | C | C | C | C |
| PS3 | 906 | T/C | T/C | T/C | T/C | T | T | T | T | T | T | T | T | T | T |
| PS4 | 913 | A | A | A | A | A | A | A | A | A | A | A | A/T | A | A |
| PS5 | 1077 | C | C | C | C | C | C | C | C | C | C | C | C | C | C |
| PS6 | 1468 | T | T | T/C | T | T | T/C | T | T | T | T | T | T | T | T |
| PS7 | 1474 | C | C | C | C | C | C | C/A | C | C | C | C | C | C | C |
| PS8 | 1610 | C/T | C/T | C | C/T | C | C | C/T | T | T | T | T | T | T | T |
| PS9 | 2422 | A | A | A | A | A | A | A | A | A | A | A | A | A | A |
| PS10 | 2738 | A | A | A | A | A | A | A | A | A/G | A | A | A | G | A/G |
| PS11 | 2789 | G | G | G | G | G | G | G | G | G | G | G/A | G | G | G/A |
| PS12 | 2934 | T | T | T/C | T | T | T/C | T | T | T | T | T | T | T | T |
| PS13 | 3000 | G | G | G | G | G | G | G | G | G | G | G/A | G | G | G/A |
| PS14 | 3044 | G | G | G | G | G | G | G | G | G | G | G | G | G | G |
| PS15 | 4552 | G/A | G | G | G/A | G | G | G | A | A | G/A | G/A | A | A | G/A |
| PS16 | 4822 | C | C | C | C | C | C | C | C | C | C | C | C | C | C |
| PS17 | 4999 | T | T | T | T | T | T | T | T | T | T | T | T | T | T |
| PS18 | 5077 | T/C | T | T | T/C | T | T | T | C | C | T/C | T/C | C | C | T/C |
| PS19 | 6535 | C | C | C | C | C | C | C | C | C | C | C | C | C | C |
| PS20 | 6625 | T | T | T/C | T | T | T/C | T | T | T | T | T | T | T | T |
| PS21 | 6650 | A | A | A | A/G | A | A | A | A | A/G | A | A | A | G | A/G |
| PS22 | 6714 | G | G | G | G | G | G | G | G | G | G | G | G | G | G |

(a)PS = polymorphic site;
(b)Position of PS in SEQ ID NO:1;
(c)Haplotype pairs are represented as 1^{st} haplotype/2^{nd} haplotype; with alleles of each haplotype shown 5′ to 3′ as 1^{st} polymorphism/2^{nd} polymorphism in each column;
and the frequency data in Tables 6 and 7.

32. A genome anthology for the Fc fragment of IgE, high affinity I, receptor for; alpha polypeptide (FCER1A) gene which comprises two or more FCER1A isogenes selected from the group consisting of isogenes 1-20 shown in the table immediately below, and wherein each of the isogenes comprises the regions of SEQ ID NO:1 shown in the table immediately below and wherein each of the isogenes 1-20 is further defined by the corresponding sequence of polymorphisms whose positions and identities are set forth in the table immediately below:

| Regions | PS | PS | Isogene Number(d) (Part 1) | | | | | | | | | |
|---|---|---|---|---|---|---|---|---|---|---|---|---|
| Examined(a) | Number(b) | Position(c) | 1 | 2 | 3 | 4 | 5 | 6 | 7 | 8 | 9 | 10 |
| | | | | | | | | | | | | |
|---|---|---|---|---|---|---|---|---|---|---|---|---|
| 319-1709 | PS1 | 586 | T | T | T | T | G | T | T | T | T | T |
| 319-1709 | PS2 | 657 | C | T | T | C | C | C | C | C | T | C |
| 319-1709 | PS3 | 906 | T | T | T | C | T | T | T | T | T | T |
| 319-1709 | PS4 | 913 | A | A | A | A | A | A | A | A | A | A |
| 319-1709 | PS5 | 1077 | C | C | C | C | C | C | C | C | C | C |
| 319-1709 | PS6 | 1468 | T | T | T | T | T | T | T | T | T | T |
| 319-1709 | PS7 | 1474 | C | C | C | C | C | C | C | C | C | C |
| 319-1709 | PS8 | 1610 | C | C | C | C | C | T | T | T | C | T |
| 2351-3067 | PS9 | 2422 | A | A | A | A | A | A | A | A | G | A |
| 2351-3067 | PS10 | 2738 | A | A | A | A | A | A | G | A | A | A |
| 2351-3067 | PS11 | 2789 | G | G | G | G | G | G | G | G | G | A |
| 2351-3067 | PS12 | 2934 | T | T | T | T | T | T | T | T | T | T |
| 2351-3067 | PS13 | 3000 | G | G | G | G | G | G | G | G | G | A |
| 2351-3067 | PS14 | 3044 | G | G | G | G | G | G | G | G | G | G |
| 4359-5177 | PS15 | 4552 | G | G | G | G | G | A | A | G | G | G |
| 4359-5177 | PS16 | 4822 | C | C | C | C | C | C | C | C | C | C |
| 4359-5177 | PS17 | 4999 | T | C | C | T | T | T | T | T | C | T |
| 4359-5177 | PS18 | 5077 | T | T | T | T | T | C | C | T | T | T |
| 6200-7073 | PS19 | 6535 | C | C | C | C | C | C | C | C | C | C |
| 6200-7073 | PS20 | 6625 | T | T | T | T | T | T | T | T | T | T |
| 6200-7073 | PS21 | 6650 | A | A | A | A | A | A | G | A | A | A |
| 6200-7073 | PS22 | 6714 | G | G | A | G | G | G | G | G | G | G |
| Regions | PS | PS | Isogene Number(d) (Part 2) | | | | | | | | | |
|---|---|---|---|---|---|---|---|---|---|---|---|---|
| Examined(a) | Number(b) | Position(c) | 11 | 12 | 13 | 14 | 15 | 16 | 17 | 18 | 19 | 20 |
| | | | | | | | | | | | | |
|---|---|---|---|---|---|---|---|---|---|---|---|---|
| 319-1709 | PS1 | 586 | G | T | T | T | T | T | T | T | T | T |
| 319-1709 | PS2 | 657 | C | C | C | T | C | C | C | C | T | T |
| 319-1709 | PS3 | 906 | T | T | T | T | T | T | T | T | T | T |
| 319-1709 | PS4 | 913 | A | A | A | A | A | A | A | T | A | A |
| 319-1709 | PS5 | 1077 | C | C | C | C | C | C | C | C | C | A |
| 319-1709 | PS6 | 1468 | C | T | T | T | T | T | T | T | T | T |
| 319-1709 | PS7 | 1474 | C | C | C | C | A | C | C | C | C | C |
| 319-1709 | PS8 | 1610 | C | C | T | T | T | C | T | T | C | T |
| 2351-3067 | PS9 | 2422 | A | A | A | A | A | A | A | A | A | A |
| 2351-3067 | PS10 | 2738 | A | A | A | A | A | A | G | A | A | A |
| 2351-3067 | PS11 | 2789 | G | G | G | G | G | G | G | G | G | G |
| 2351-3067 | PS12 | 2934 | C | T | T | T | T | T | T | T | T | T |
| 2351-3067 | PS13 | 3000 | G | G | G | G | G | G | G | G | G | G |
| 2351-3067 | PS14 | 3044 | G | G | G | G | G | A | G | G | G | G |
| 4359-5177 | PS15 | 4552 | G | G | A | G | G | G | G | A | G | A |
| 4359-5177 | PS16 | 4822 | C | C | C | C | C | C | C | C | T | C |
| 4359-5177 | PS17 | 4999 | T | T | T | C | T | T | T | T | C | C |
| 4359-5177 | PS18 | 5077 | T | T | C | T | T | T | T | C | T | C |
| 6200-7073 | PS19 | 6535 | C | A | C | C | C | A | C | C | C | C |
| 6200-7073 | PS20 | 6625 | C | T | T | T | T | T | T | T | T | T |
| 6200-7073 | PS21 | 6650 | A | A | G | A | A | A | A | A | A | A |
| 6200-7073 | PS22 | 6714 | G | G | G | A | G | G | G | G | A | G |

(a)Region examined represents the nucleotide positions defining the start and stop positions within SEQ ID NO: 1 of the regions sequenced;
(b)PS = polymorphic site;
(c)IPosition of PS within SEQ ID NO:1;
(d)Alleles for FCER1A isogenes are presented 5′ to 3′ in each column.


SEQUENCE LISTING (114 sequences)

SEQ ID NO: 1 - DNA, 7372 bp, Homo sapiens
  allele (586)..(586): PS1 Polymorphic base T or G
aaacagaagaattagtaaaggaatcctggagaaagcccctgctgtgtatttaaaggagaaagggagatcatgttgggaaattataatattaaaagtaaacaaaagctaggaagtaaaataaaataaattatatggcctagatccccataagtaatggtttaacttctgccttcctgtgttctgagccagattagggcacagtagagaaagaggagtctctgaaaatgtttccaatttcgctggtcagacagcggatcatcagtgaatcagatgaaaatttgtggatttatgcactaactgatcagcaggaaattaaacaagaaaagcgttggtagctctggtgaatcccaaaagaatttggcagttgctagccatgctcctgaatatgtataaacagtacatcatatgactaagagtttgacttaggggttagattttatgtgtttgaaccccaaattagttatttaatagttggcaccccaaaacaagttacttaacctcactaagattcagttttcctgtttataaaatgtagatagtgatagtatgtactttataggattattgtgaaaaataaatgaaatakcagatttatttaggataacacctggcatatgtttggtattcagtaattagttgctgctgttttattctgcyctcccttgcatcccacttttctaagttgtaaactaaatagttgtacacagattgacagattaagaaaggcttgtgattgtgctagacctatgcctctctctcaccagattccaggtgtatatgtggaggtgggatagggagtggagtaagtgggtaaatattaaattgcccagttgggcaccatcctgaatattatctctaaagaaagaagcaaaaccaggcacagctgatgggttaaccagatatgayacagaawacatttccttctgctttttggttttaagcctatatttgaagccttagatctctccagcacagtaagcaccaggagtccatgaagaagatggctcctgccatggaatcccctactctactgtgtgtagccttactgttcttcggtaagtagagattcaattaccmctcccagggaggcccaaatgaatttggggagcagctggggtaggaacctttactgtgggtggtgactttttctaggacatgtgcaaactattgggcatttcccagggactctgtagtggagccaagctagaaagcagaggcaagtgggctgagcaacacctaaggaggaagccagactgaaagcttggttccttgcatttgctctggcatcttccagagtgcaaatttcctaccaaggtaatgagggtagaggagagaaagaagctctttcttcccctgattctcattcctgaaaagacggttggtccttaaaattccatggatgtagatcttatccccacacccagattctagtcctctggagataaagaagactgctggacactaatgyatcctmtctggacttttgcagctccagatggcgtgttagcaggtgagtcctctgttcttgttcccttggtgtatcaacatgtctgggcattgctttcctctcactattttcttcgtcccatcacttctgctttctaatgagyatgaatctgttccttggccagactactttccctctccaccttgccttgtctttctttttttccctgattcattgcattctctcaagtcattctctcctctgttttagtcaataaccatgtctgttgcacatatacatgtctcattctctctcctagacactttggcatgatctcgctcaataattacattattattattattgccattttataattgaggatgctgaaactcagtgattttctggtggttacatggctaaggaactggatttcaacgtaagttccttggatctaagtccagttctcttctgactatatcacccttttgttatcaccatgtatctacttctttggtctctgttcaaatttgcactacatccccttgttccaggaagccattcaagactgactttcttagtgcctctcactactttctggaactgacatatgtttttcactctgtatatacttacaattaaatagtcataaatattcagagcttggagaaaccttatatttcatccagtccagtaaatttatccatccataattcactcattcattcacataataaatatttaatgtaacaatggttgaacatggcagacagtgtttctacctcaaaagagattgcagtcctcatttacagatactgaattgaaattaacagaagtagagtgagtcagctcaaatcacatagtgaattggtttctttgtttttaaatctcctgcatatgtgtcctgtctttctccctgtgttgggcgttccctggggcaccaatactaatttctccttcccctagaaatcaaarcagggtcttatcaccaacagaataaggacaggttgaccactgattgtcagaatattgcttcgtttgtacttttaagcctagacagttttcaatgactttttttctctctacatgtcttttcatatttttatcttcttgaagtccctcagaaacctaaggtctccttgaaccctccatggaatagaatatttaaaggagagaatgtgactcttacatgtaatgggaacaatttctttgaagtcagttccaccaaatggttccacaatggcagcctttcagaagagacaaattcaagtttgaatattgtgaatgccaratttgaagacagtggagaatacaaatgtcagcaccaacaagttaatgagartgaacctgtgtacctggaagtcttcagtggtaagttccagggatatggaaatacagatctctcatgtgagggatggctcatctgaagatgggaaaaaacaggttattccaagggttaggacaccagagtgggattcaaggcctcycatttttaagacccctgcattggctgggcacagtggctcacgcctgtaatcccagcactttgggargctgaggcaggtggatcacgaggtcaggagatcgagaccatccrgctaacatggtgaaaccccatctctgctaaaaaatatatatatataaaattagccgggcgtagtggtgggcacctgtagtcccaggtactcgggaggctgaggcaggagaatggtgtgaacccaggaggtggaggttgcagtgagctgagatcacgccactgccctccagcctgggctacagagcaagactccgtctcaaaaaataaataaataaataaaaaagacccctgcatctcttttcttctacccccttcccttttgattacttgtatgccttctttcaatattctagtcatctctcaatattattcctccaccctattttcctctatcttttctgcctagattcaggtatatattatgtggtcaaacagcatgacatatatgtgaacatttcaaagagctgtgtatctggaataggatcaaaaggtttgacttaaagttttgctctgcataatccatatggcaggacctgaatattaggttgtactcttcgttatgaaacatatctgggtacatttccttatgtcctctgttgttacttaagaacacatatttcatgcttgtttcatttttatcactcctactgccaacaaatagcatagcatgcttaggcacatgtggcttaattagcaaatgttgaataaacaaattaatgattttgaatagtgaccaataggtctcttttatactctatatttttctcttgagtgaaaaaaaatgtttcaacctccatatgtaaattccaaacacaaactaaagcaatgtagaatagcttctttattccctggagtaggttctagagaagtcctaaaggattggtcctaaattaattatgcttattatgctagcgatatttcctttcaaaattctcctttaatgaatgctttttaatttttacaaaagcattaaccatagaatgtgattcttgtctttcactgactcattagtgacaaatatttgttgagtacctaccaactcctaagtattgctaccaactcctaaatactgtgttgggcattcagaatagaatgtagaactagacagggtccctgacttcttggagcacagagcagtatgggaagaggacattaaataaagaattacataagtaattaatttaaattatacatgttttgaagaagtttttttttgacaactataattaacactagaactgggaagtttctataaggtaagagaggacaaaatagacactctcctaagctaaaattcccaagaaagactgtttattttcccctaactaactagaactagcaacagaagatctgaaaggaattctggctttcaagtgttccatgtatggactcatcagggaggtccgagaggctttgtggccccagactgacttttcaggaggggaaaggatttatcaatacacaagacaggctctaagcattattttgtgccctttaaaaatccactttatgagccaaaaagtgagttaatgataattcatagtttctgacacatgctctatgcgtgrctctcttttctctattcattctctctctcttcatttattgttaaataaataatgtaatgaatgttcttcagactggctgctccttcaggcctctgctgaggtggtgatggagggccagcccctcttcctcaggtgccatggttggaggaactgggatgtgtacaaggtgatctattataaggatggtgaagctctcaagtactggtatgagaaccacaacatctccattacaaatgccacagttgaagacagtggaacctactactgtaygggcaaagtgtggcagctggactatgagtctgagcccctcaacattactgtaataaaaggtgagttggtaaaggaaaggaaaagcatccatagcaggggaaggaagagagaacttctgagcctgagcagttgcagcttgtagaaggggggcacctgtgatacactggaaagcctacyagacttgcaatgaggagacctgggtgatagtatatatctcaatctctgtttcaaagccttgacttgttaaatggtgayagtaatacctgcttgcactatgaaatttttatgaagattaatgtggtaatatttgtgaaatgactttgtaaactgttaagcactacccaagcataacagattgtgattactattttgatctcaaagtcatctgttgctcctgggggaacacttatatttatcaaattgaaaaaaagtttcaaagttgaatgaagaaaggatataaagagcttgaggagcccattccagcttaggagggctgggaaaggaaaccagcaagtcagtaagctgtgtgcctgtgtattgagggaggagggaatggacttgatatggagagggtagggaggtggactgcctctatggcctgtaagaaaaactgctctctccaaactctttataagagagggagcctgtgaagtattcacttttgaaggagaaagttagacttttccttcacacactttgtacataataatgtttaaaaaagcatgaggtcaaaatacataattaagtcctagcagttctctgttaactaatttgagactgaagtgctatgtacttgtctctaggcttccagtatcttcatctgtaaaacagaatatttggtctagattccattagaatcatttgataacttaaaaaatatattgatgctcatgtctcatttcttgagattctgatttaattggtttggggtgcagcctgggtatacgtatttttcataggtctttcacataatggtaatgggtagccaatattgagaatcacttgtctaggtgatctttaaatgatttctggatgtaatattctgaggctctataatttgagactaatcacaaaaatcggtacagtttataaacagactaacagaaccacaaaataatagaattggaaggcaatttaactagtgcaatttcttcattttgcctaacaggcatgtaagaaatgatgattgattgagtaataggcattgatgacccctgtcctcactttgtcccctttccaccccttaattatatgtgaattctggtcttgtcatttcgaataaggggtttatctttcctattgtcttcccctctgggcacggcacactggctactggagttaagaggaaatgcttaggactccctgtggctccagggagcaccaacagagcaactcaacctagtgttaatctgagtgttttctctgtgcttctggatgccacatcacgctaaaaatgaaggacaaagcttggtctttctcttagggaggatgaaactctgaacctcatttttcagttcccaagatgaattatgtttctcattgcatctgtgttccactacagctccgcgtgagaagtactggctacaattttttatcccattgttggtggtgattctgtttgctgtggacacaggattatttatctcaactcagcagcaggtcacatttctcttgaagattaagagaaccaggaaaggcttcagacttctgaamccacatcctaagccaaaccccaaaaacaactgatataattactcaagaaatatttgcaacattagtttttttccagcatcagcaattgcyactcaattgtcaaacacagcttgcratatacatagaaacgtctgtgctcaaggatttatagaaatgcttcattaaactgagtgaaactrgttaagtggcatgtaatagtaagtgctcaattaacattggttgaataaatgagagaatgaatagattcatttattagcatttgtaaaagagatgttcaatttcaataaaataaatataaaaccatgtaacagaatgcttctgagtattcaaggcttgctagtttgtttgtttgttttctactaaaggcaaggaccatgaagttctagattggaaatgtcctctcttgactattgcaagtgcgatctaggaatgaaaagacataggaggatgccagtgaggtggatcatttttatgcttcttcttcagcttactaaatatgaactttcagttcttggcagaatcagggacagtctcaagacataggactctcaggatgaagtagagtccaggattcctctgtgattgttttgcccctcccaaatttatatcttgaacttatgtcttgtatctttatacagcacctgaaccaagcattttggagaaattccagctaataataataaccaaaaccttcggctctgaaaacagtccaggactgaataagatcttgggcaaaagaactagacagttttggtttattttccctttcattttatgtcttcatcatagtcattggaggctcattcttcttgtcatggagtaaatgggattaaagtt

SEQ ID NO: 2 - DNA, 774 bp, Homo sapiens
atggctcctgccatggaatcccctactctactgtgtgtagccttactgttcttcgctccagatggcgtgttagcagtccctcagaaacctaaggtctccttgaaccctccatggaatagaatatttaaaggagagaatgtgactcttacatgtaatgggaacaatttctttgaagtcagttccaccaaatggttccacaatggcagcctttcagaagagacaaattcaagtttgaatattgtgaatgccaaatttgaagacagtggagaatacaaatgtcagcaccaacaagttaatgagagtgaacctgtgtacctggaagtcttcagtgactggctgctccttcaggcctctgctgaggtggtgatggagggccagcccctcttcctcaggtgccatggttggaggaactgggatgtgtacaaggtgatctattataaggatggtgaagctctcaagtactggtatgagaaccacaacatctccattacaaatgccacagttgaagacagtggaacctactactgtacgggcaaagtgtggcagctggactatgagtctgagcccctcaacattactgtaataaaagctccgcgtgagaagtactggctacaattttttatcccattgttggtggtgattctgtttgctgtggacacaggattatttatctcaactcagcagcaggtcacatttctcttgaagattaagagaaccaggaaaggcttcagacttctgaacccacatcctaagccaaaccccaaaaacaactga

SEQ ID NO: 3 - protein, 257 aa, Homo sapiens
MAPAMESPTLLCVALLFFAPDGVLAVPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKAPREKYWLQFFIPLLVVILFAVDTGLFISTQQQVTFLLKIKRTRKGFRLLNPHPKPNPKNN

SEQ ID NO: 4 - DNA, 15 bp, Homo sapiens
tgaaatakcagattt

SEQ ID NO: 5 - DNA, 15 bp, Homo sapiens
attctgcyctccctt

SEQ ID NO: 6 - DNA, 15 bp, Homo sapiens
gatatgayacagaaa

SEQ ID NO: 7 - DNA, 15 bp, Homo sapiens
tacagaawacatttc

SEQ ID NO: 8 - DNA, 15 bp, Homo sapiens
aattaccmctcccag

SEQ ID NO: 9 - DNA, 15 bp, Homo sapiens
actaatgyatcctct

SEQ ID NO: 10 - DNA, 15 bp, Homo sapiens
gtatcctmtctggac

SEQ ID NO: 11 - DNA, 15 bp, Homo sapiens
taatgagyatgaatc

SEQ ID NO: 12 - DNA, 15 bp, Homo sapiens
aatcaaarcagggtc

SEQ ID NO: 13 - DNA, 15 bp, Homo sapiens
aatgccaratttgaa

SEQ ID NO: 14 - DNA, 15 bp, Homo sapiens
aatgagartgaacct

SEQ ID NO: 15 - DNA, 15 bp, Homo sapiens
aggcctcycattttt

SEQ ID NO: 16 - DNA, 15 bp, Homo sapiens
tttgggargctgagg

SEQ ID NO: 17 - DNA, 15 bp, Homo sapiens
accatccrgctaaca

SEQ ID NO: 18 - DNA, 15 bp, Homo sapiens
atgcgtgrctctctt

SEQ ID NO: 19 - DNA, 15 bp, Homo sapiens
tactgtaygggcaaa

SEQ ID NO: 20 - DNA, 15 bp, Homo sapiens
agcctacyagacttg

SEQ ID NO: 21 - DNA, 15 bp, Homo sapiens
atggtgayagtaata

SEQ ID NO: 22 - DNA, 15 bp, Homo sapiens
ttctgaamccacatc

SEQ ID NO: 23 - DNA, 15 bp, Homo sapiens
caattgcyactcaat

SEQ ID NO: 24 - DNA, 15 bp, Homo sapiens
agcttgcratataca

SEQ ID NO: 25 - DNA, 15 bp, Homo sapiens
tgaaactrgttaagt

SEQ ID NO: 26 - DNA, 15 bp, Homo Sapiens
aataaatgaaatakc

SEQ ID NO: 27 - DNA, 15 bp, Homo sapiens
ctaaataaatctgmt

SEQ ID NO: 28 - DNA, 15 bp, Homo Sapiens
tgttttattctgcyc

SEQ ID NO: 29 - DNA, 15 bp, Homo sapiens
ggatgcaagggagrg

SEQ ID NO: 30 - DNA, 15 bp, Homo Sapiens
taaccagatatgaya

SEQ ID NO: 31 - DNA, 15 bp, Homo sapiens
aaatgttttctgtrt

SEQ ID NO: 32 - DNA, 15 bp, Homo Sapiens
atatgatacagaawa

SEQ ID NO: 33 - DNA, 15 bp, Homo sapiens
cagaaggaaatgtwt

SEQ ID NO: 34 - DNA, 15 bp, Homo Sapiens
agattcaattaccmc

SEQ ID NO: 35 - DNA, 15 bp, Homo sapiens
gcctccctgggagkg

SEQ ID NO: 36 - DNA, 15 bp, Homo Sapiens
ctggacactaatgya

SEQ ID NO: 37 - DNA, 15 bp, Homo sapiens
gtccagagaggatrc

SEQ ID NO: 38 - DNA, 15 bp, Homo Sapiens
actaatgtatcctmt

SEQ ID NO: 39 - DNA, 15 bp, Homo sapiens
gcaaaagtccagaka

SEQ ID NO: 40 - DNA, 15 bp, Homo Sapiens
gctttctaatgagya

SEQ ID NO: 41 - DNA, 15 bp, Homo sapiens
ggaacagattcatrc

SEQ ID NO: 42 - DNA, 15 bp, Homo Sapiens
cctagaaatcaaarc

SEQ ID NO: 43 - DNA, 15 bp, Homo sapiens
tgataagaccctgyt

SEQ ID NO: 44 - DNA, 15 bp, Homo Sapiens
attgtgaatgccara

SEQ ID NO: 45 - DNA, 15 bp, Homo sapiens
actgtcttcaaatyt

SEQ ID NO: 46 - DNA, 15 bp, Homo Sapiens
caagttaatgagart

SEQ ID NO: 47 - DNA, 15 bp, Homo sapiens
gtacacaggttcayt

SEQ ID NO: 48 - DNA, 15 bp, Homo Sapiens
gattcaaggcctcyc

SEQ ID NO: 49 - DNA, 15 bp, Homo sapiens
ggtcttaaaaatgrg

SEQ ID NO: 50 - DNA, 15 bp, Homo Sapiens
cagcactttgggarg

SEQ ID NO: 51 - DNA, 15 bp, Homo sapiens
cacctgcctcagcyt

SEQ ID NO: 52 - DNA, 15 bp, Homo Sapiens
atcgagaccatccrg

SEQ ID NO: 53 - DNA, 15 bp, Homo sapiens
tcaccatgttagcyg

SEQ ID NO: 54 - DNA, 15 bp, Homo Sapiens
tgctctatgcgtgrc

SEQ ID NO: 55 - DNA, 15 bp, Homo sapiens
agagaaaagagagyc

SEQ ID NO: 56 - DNA, 15 bp, Homo Sapiens
acctactactgtayg

SEQ ID NO: 57 - DNA, 15 bp, Homo sapiens
ccacactttgcccrt

SEQ ID NO: 58 - DNA, 15 bp, Homo Sapiens
ctggaaagcctacya

SEQ ID NO: 59 - DNA, 15 bp, Homo sapiens
tcattgcaagtctrg

SEQ ID NO: 60 - DNA, 15 bp, Homo Sapiens
tgttaaatggtgaya

SEQ ID NO: 61 - DNA, 15 bp, Homo sapiens
agcaggtattactrt

SEQ ID NO: 62 - DNA, 15 bp, Homo Sapiens
tcagacttctgaamc

SEQ ID NO: 63 - DNA, 15 bp, Homo sapiens
gcttaggatgtggkt

SEQ ID NO: 64 - DNA, 15 bp, Homo Sapiens
catcagcaattgcya

SEQ ID NO: 65 - DNA, 15 bp, Homo sapiens
ttgacaattgagtrg

SEQ ID NO: 66 - DNA, 15 bp, Homo Sapiens
aaacacagcttgcra

SEQ ID NO: 67 - DNA, 15 bp, Homo sapiens
tttctatgtatatyg

SEQ ID NO: 68 - DNA, 15 bp, Homo Sapiens
actgagtgaaactrg

SEQ ID NO: 69 - DNA, 15 bp, Homo sapiens
catgccacttaacya

SEQ ID NO: 70 - DNA, 10 bp, Homo sapiens
aaatgaaata

SEQ ID NO: 71 - DNA, 10 bp, Homo sapiens
aataaatctg

SEQ ID NO: 72 - DNA, 10 bp, Homo sapiens
tttattctgc

SEQ ID NO: 73 - DNA, 10 bp, Homo sapiens
tgcaagggag

SEQ ID NO: 74 - DNA, 10 bp, Homo sapiens
ccagatatga

SEQ ID NO: 75 - DNA, 10 bp, Homo sapiens
tgttttctgt

SEQ ID NO: 76 - DNA, 10 bp, Homo sapiens
tgatacagaa

SEQ ID NO: 77 - DNA, 10 bp, Homo sapiens
aaggaaatgt

SEQ ID NO: 78 - DNA, 10 bp, Homo sapiens
ttcaattacc

SEQ ID NO: 79 - DNA, 10 bp, Homo sapiens
tccctgggag

SEQ ID NO: 80 - DNA, 10 bp, Homo sapiens
gacactaatg

SEQ ID NO: 81 - DNA, 10 bp, Homo sapiens
cagagaggat

SEQ ID NO: 82 - DNA, 10 bp, Homo sapiens
aatgtatcct

SEQ ID NO: 83 - DNA, 10 bp, Homo sapiens
aaagtccaga

SEQ ID NO: 84 - DNA, 10 bp, Homo sapiens
ttctaatgag

SEQ ID NO: 85 - DNA, 10 bp, Homo sapiens
acagattcat

SEQ ID NO: 86 - DNA, 10 bp, Homo sapiens
agaaatcaaa

SEQ ID NO: 87 - DNA, 10 bp, Homo sapiens
taagaccctg

SEQ ID NO: 88 - DNA, 10 bp, Homo sapiens
gtgaatgcca

SEQ ID NO: 89 - DNA, 10 bp, Homo sapiens
gtcttcaaat

SEQ ID NO: 90 - DNA, 10 bp, Homo sapiens
gttaatgaga

SEQ ID NO: 91 - DNA, 10 bp, Homo sapiens
cacaggttca

SEQ ID NO: 92 - DNA, 10 bp, Homo sapiens
tcaaggcctc

SEQ ID NO: 93 - DNA, 10 bp, Homo sapiens
cttaaaaatg

SEQ ID NO: 94 - DNA, 10 bp, Homo sapiens
cactttggga

SEQ ID NO: 95 - DNA, 10 bp, Homo sapiens
ctgcctcagc

SEQ ID NO: 96 - DNA, 10 bp, Homo sapiens
gagaccatcc

SEQ ID NO: 97 - DNA, 10 bp, Homo sapiens
ccatgttagc

SEQ ID NO: 98 - DNA, 10 bp, Homo sapiens
tctatgcgtg

SEQ ID NO: 99 - DNA, 10 bp, Homo sapiens
gaaaagagag

SEQ ID NO: 100 - DNA, 10 bp, Homo sapiens
tactactgta

SEQ ID NO: 101 - DNA, 10 bp, Homo sapiens
cactttgccc

SEQ ID NO: 102 - DNA, 10 bp, Homo sapiens
gaaagcctac

SEQ ID NO: 103 - DNA, 10 bp, Homo sapiens
ttgcaagtct

SEQ ID NO: 104 - DNA, 10 bp, Homo sapiens
taaatggtga

SEQ ID NO: 105 - DNA, 10 bp, Homo sapiens
aggtattact

SEQ ID NO: 106 - DNA, 10 bp, Homo sapiens
gacttctgaa

SEQ ID NO: 107 - DNA, 10 bp, Homo sapiens
taggatgtgg

SEQ ID NO: 108 - DNA, 10 bp, Homo sapiens
cagcaattgc

SEQ ID NO: 109 - DNA, 10 bp, Homo sapiens
acaattgagt

SEQ ID NO: 110 - DNA, 10 bp, Homo sapiens
cacagcttgc

SEQ ID NO: 111 - DNA, 10 bp, Homo sapiens
ctatgtatat

SEQ ID NO: 112 - DNA, 10 bp, Homo sapiens
gagtgaaact

SEQ ID NO: 113 - DNA, 10 bp, Homo sapiens
gccacttaac

SEQ ID NO: 114 - DNA, 2640 bp, Homo sapiens
  allele (30)..(30): PS1 Polymorphic base T or G
taggattattgtgaaaaataaatgaaatakcagatttatttaggataacacctggcatatnnnnnnnnnnnnnnnnnnnnnnnnnnnnnnnnnnnnnnnnnnnnnnnnnnnnnnnnnnnnagtaattagttgctgctgttttattctgcyctcccttgcatcccacttttctaagttgtannnnnnnnnnnnnnnnnnnnnnnnnnnnnnnnnnnnnnnnnnnnnnnnnnnnnnnnnnnnggcacagctgatgggttaaccagatatgayacagaaaacatttccttctgctttttggttnnnnnnnnnnnnnnnnnnnnnnnnnnnnnnnnnnnnnnnnnnnnnnnnnnnnnnnnnnnnctgatgggttaaccagatatgatacagaawacatttccttctgctttttggttttaagccnnnnnnnnnnnnnnnnnnnnnnnnnnnnnnnnnnnnnnnnnnnnnnnnnnnnnnnnnnnngttcttcggtaagtagagattcaattaccmctcccagggaggcccaaatgaatttggggannnnnnnnnnnnnnnnnnnnnnnnnnnnnnnnnnnnnnnnnnnnnnnnnnnnnnnnnnnngagataaagaagactgctggacactaatgyatcctctctggacttttgcagctccagatgnnnnnnnnnnnnnnnnnnnnnnnnnnnnnnnnnnnnnnnnnnnnnnnnnnnnnnnnnnnnaagaagactgctggacactaatgtatcctmtctggacttttgcagctccagatggcgtgtnnnnnnnnnnnnnnnnnnnnnnnnnnnnnnnnnnnnnnnnnnnnnnnnnnnnnnnnnnnntcgtcccatcacttctgctttctaatgagyatgaatctgttccttggccagactactttcnnnnnnnnnnnnnnnnnnnnnnnnnnnnnnnnnnnnnnnnnnnnnnnnnnnnnnnnnnnnactaatttctccttcccctagaaatcaaarcagggtcttatcaccaacagaataaggacannnnnnnnnnnnnnnnnnnnnnnnnnnnnnnnnnnnnnnnnnnnnnnnnnnnnnnnnnnnaaattcaagtttgaatattgtgaatgccaratttgaagacagtggagaatacaaatgtcannnnnnnnnnnnnnnnnnnnnnnnnnnnnnnnnnnnnnnnnnnnnnnnnnnnnnnnnnnncaaatgtcagcaccaacaagttaatgagartgaacctgtgtacctggaagtcttcagtggnnnnnnnnnnnnnnnnnnnnnnnnnnnnnnnnnnnnnnnnnnnnnnnnnnnnnnnnnnnntaggacaccagagtgggattcaaggcctcycatttttaagacccctgcattggctgggcannnnnnnnnnnnnnnnnnnnnnnnnnnnnnnnnnnnnnnnnnnnnnnnnnnnnnnnnnnnctcacgcctgtaatcccagcactttgggargctgaggcaggtggatcacgaggtcaggagnnnnnnnnnnnnnnnnnnnnnnnnnnnnnnnnnnnnnnnnnnnnnnnnnnnnnnnnnnnnatcacgaggtcaggagatcgagaccatccrgctaacatggtgaaaccccatctctgctaannnnnnnnnnnnnnnnnnnnnnnnnnnnnnnnnnnnnnnnnnnnnnnnnnnnnnnnnnnncatagtttctgacacatgctctatgcgtgrctctcttttctctattcattctctctctctnnnnnnnnnnnnnnnnnnnnnnnnnnnnnnnnnnnnnnnnnnnnnnnnnnnnnnnnnnnnagttgaagacagtggaacctactactgtaygggcaaagtgtggcagctggactatgagtcnnnnnnnnnnnnnnnnnnnnnnnnnnnnnnnnnnnnnnnnnnnnnnnnnnnnnnnnnnnngggcacctgtgatacactggaaagcctacyagacttgcaatgaggagacctgggtgatagnnnnnnnnnnnnnnnnnnnnnnnnnnnnnnnnnnnnnnnnnnnnnnnnnnnnnnnnnnnntttcaaagccttgacttgttaaatggtgayagtaatacctgcttgcactatgaaatttttnnnnnnnnnnnnnnnnnnnnnnnnnnnnnnnnnnnnnnnnnnnnnnnnnnnnnnnnnnnnagaaccaggaaaggcttcagacttctgaamccacatcctaagccaaaccccaaaaacaacnnnnnnnnnnnnnnnnnnnnnnnnnnnnnnnnnnnnnnnnnnnnnnnnnnnnnnnnnnnnattagtttttttccagcatcagcaattgcyactcaattgtcaaacacagcttgcaatatannnnnnnnnnnnnnnnnnnnnnnnnnnnnnnnnnnnnnnnnnnnnnnnnnnnnnnnnnnnttgctactcaattgtcaaacacagcttgcratatacatagaaacgtctgtgctcaaggatnnnnnnnnnnnnnnnnnnnnnnnnnnnnnnnnnnnnnnnnnnnnnnnnnnnnnnnnnnnnagaaatgcttcattaaactgagtgaaactrgttaagtggcatgtaatagtaagtgctcaannnnnnnnnnnnnnnnnnnnnnnnnnnnnnnnnnnnnnnnnnnnnnnnnnnnnnnnnnnn